SEQUENCE LISTING (7 sequences)

SEQ ID NO: 1 - protein, 245 aa, Homo sapiens
MTSSYGHVLERQPALGGRLDSPGNLDTLQAKKNFSVSHLLDLEEAGDMVAAQADENVGEAGRSLLESPGLTSGSDTPQQDNDQLNSEEKKKRKQRRNRTTFNSSQLQALERVFERTHYPDAFVREDLARRVNLTEARVQVWFQNRRAKFRRNERAMLANKNASLLKSYSGDVTAVEQPIVPRPAPRPTDYLSWGTASPYSAMATYSATCANNSPAQGINMANSIANLRLKAKEYSLQRNQVPTVN

SEQ ID NO: 2 - protein, 217 aa, Homo sapiens
MTSSYGHVLERQPALGGRLDSPGNLDTLQAKKNFSVSHLLDLEEAGDMVAAQADENVGEAGRSLLESPGLTSGSDTPQQDNDQLNSEEKKKRKQRRNRTTFNSSQLQALERVFERTHYPDAFVREDLARRVNLTEARVQVWFQNRRAKFRRNERAMLANKNASLLKSYSGDVTAVEQPIVPRPAPRPTDYLSWGTASPYRSSSLPRCCLHEGLHNGF

SEQ ID NO: 3 - protein, 200 aa, Mus musculus
MTSSYGHVLERQPALGGRLDSPGNLDTLQAKKNFSVSHLLDLEEAGDMVAAQADESVGEAGRSLLESPGLTSGSDTPQQDNDQLNSEEKKKRKQRRNRTTFNSSQLQALERVFERTHYPDAFVREDLARRVNLTEARVQVWFQNRRAKFRRNERAMLANKNASLLKSYSGDVTAVEQPIVPRPAPRPTDYLSWGTASPYR

SEQ ID NO: 4 - protein, 245 aa, Mus musculus
MTSSYGHVLERQPALGGRLDSPGNLDTLQAKKNFSVSHLLDLEEAGDMVAAQADESVGEAGRSLLESPGLTSGSDTPQQDNDQLNSEEKKKRKQRRNRTTFNSSQLQALERVFERTHYPDAFVREDLARRVNLTEARVQVWFQNRRAKFRRNERAMLANKNASLLKSYSGDVTAVEQPIVPRPAPRPTDYLSWGTASPYSAMATYSATCANNSPAQGINMANSIANLRLKAKEYSLQRNQVPTVN

SEQ ID NO: 5 - protein, 217 aa, Mus musculus
MTSSYGHVLERQPALGGRLDSPGNLDTLQAKKNFSVSHLLDLEEAGDMVAAQADESVGEAGRSLLESPGLTSGSDTPQQDNDQLNSEEKKKRKQRRNRTTFNSSQLQALERVFERTHYPDAFVREDLARRVNLTEARVQVWFQNRRAKFRRNERAMLANKNASLLKSYSGDVTAVEQPIVPRPAPRPTDYLSWGTASPYRSSSLPRCCLHEGLHNGF

SEQ ID NO: 6 - protein, 245 aa, Mus musculus; identical to SEQ ID NO: 4

SEQ ID NO: 7 - protein, 217 aa, Mus musculus; identical to SEQ ID NO: 5

(19) United States
(12) Patent Application Publication
(10) Pub. No.: US 2026/0108480 A1
(43) Pub. Date: Apr. 23, 2026

(54) METHODS FOR INDUCING PROLIFERATION OF STEM CELLS

(71) Applicant: UNIVERSITY OF PITTSBURGH-OF THE COMMONWEALTH SYSTEM OF HIGHER EDUCATION, Pittsburgh, PA (US)

(72) Inventors: Giuseppe INTINI, Pittsburgh, PA (US); Luigi MANCINELLI, Pittsburgh, PA (US); Xue Hui GENG, Pittsburgh, PA (US)

(21) Appl. No.: 19/114,182

(22) PCT Filed: Sep. 25, 2023
(86) PCT No.: PCT/US23/75051

§ 371 Date: Mar. 21, 2025

Related U.S. Application Data
(60) Provisional application No. 63/409,489, filed on Sep. 23, 2022.
(60) Provisional application No. 63/434,141, filed on Dec. 21, 2022.

(51) Int. Cl. A61K 31/167; A61K 31/352; A61K 31/4709; A61P 19/08

(52) U.S. Cl. CPC A61K 31/167; A61K 31/352; A61K 31/4709; A61P 19/08

(57) ABSTRACT

Described herein are methods of inducing proliferation of PRX1 expressing stem cells stem cells in a subject in need thereof, the method can include administering an effective amount of niclosamide, or a pharmaceutically acceptable salt, prodrug, or derivative thereof.

### CROSS-REFERENCE TO RELATED APPLICATION

This application claims priority to U.S. Provisional Application 63/409,489, filed on Sep. 23, 2022 and U.S. Provisional Application 63/434,141, filed on Dec. 21, 2022, the contents of which are hereby incorporated in their entirety.

### STATEMENT REGARDING FEDERALLY SPONSORED RESEARCH OR DEVELOPMENT

This invention was made with government support under Grant No. DE026155 awarded by National Institutes of Health. The government has certain rights in the invention.

### REFERENCE TO SEQUENCE LISTING

The Sequence Listing submitted Sep. 25, 2023, as a text filed named “10504-086WO1_09_25_2023_SEQUENCE_LISTING.xml” created Sep. 25, 2023, and having a file size of 8,673 bytes is hereby incorporated by reference pursuant to 37 C.F.R. § 1.52(e)(5).

### BACKGROUND

One significant barrier separates the use of skeletal stem cells (SSCs) from successful therapies: the lack of strategies to endogenously harness them. These “endogenous strategies”, by circumventing the significant biological, regulatory, and financial barriers that are associated to the current therapeutic protocols based on the ex vivo expansion of stem cells, may represent a significant therapeutic advance.

There is a need for compounds that can induce proliferation of PRX1 expressing stem cells.

The compositions and methods disclosed herein address these and other needs.

### SUMMARY

Provided herein are methods of inducing proliferation of PRX1 expressing stem cells in a subject in need thereof, the method comprising administering an effective amount of niclosamide, or a pharmaceutically acceptable salt, prodrug, or derivative thereof; or a pharmaceutical composition described herein. In some embodiments, the PRX1 expressing stem cells can include, but are not limited to, skeletal stem cells (SSCs), pulp stem cells, periodontal stem cells, ligament stem cells, adipose stem cells, and dermis stem cells. PRX1 (or PRRX1) is a transcription factor expressed during embryogenesis and postnatally.

Described herein are also methods of inducing proliferation of skeletal stem cells (SSCs) in a subject in need thereof, the method comprising administering an effective amount of niclosamide, or a pharmaceutically acceptable salt, prodrug, or derivative thereof; or a pharmaceutical composition described herein.

In some embodiments, inducing proliferation of skeletal stem cells (SSCs) is associated with bone regeneration and/or bone rejuvenation. In some embodiments, the subject can be a healthy subject. In some embodiments, the subject can have a bone-related disease, bone fracture, bone injury, bone abnormality (e.g., bone developmental abnormality), or any combination thereof. In some embodiments, the skeletal stem cells (SSCs) can be PRX1 expressing skeletal stem cells (SSCs).

Described herein are also methods for bone rejuvenation and/or bone regeneration in a subject in need thereof, the method comprising administering an effective amount of niclosamide, or a pharmaceutically acceptable salt, prodrug, or derivative thereof.

Described herein are also methods of treating a bone disease, bone fracture, bone injury, bone abnormality, or any combination thereof, the method comprising administering an effective amount of niclosamide, or a pharmaceutically acceptable salt, prodrug, or derivative thereof.

In some embodiments, the methods can further include administering an effective amount of pyrvinium, or a pharmaceutically acceptable salt, prodrug, or derivative thereof; quercetin, or a pharmaceutically acceptable salt, prodrug, or derivative thereof; or any combination thereof.

In some embodiments, the method can include administering an effective amount of a pharmaceutical composition comprising niclosamide, or a pharmaceutically acceptable salt, prodrug, or derivative thereof; and a pharmaceutically acceptable carrier. In some embodiments, the pharmaceutical composition can further include an effective amount of pyrvinium, or a pharmaceutically acceptable salt, prodrug, or derivative thereof; quercetin, or a pharmaceutically acceptable salt, prodrug, or derivative thereof; or any combination thereof.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-1H. Single cell RNA sequencing analysis of 4 day old, 2 month old, 4 month old, and 14 month old calvarial sutures. (1A, 1B, 1C, and 1D) Uniform Manifold Approximation and Projection (UMAP) plot showing unbiased graph-based clusters distribution of all cell populations in sutures isolated from 4 day old (1A), 2 month old (1B), 4 months old (1C), and 14 months old (1D) mice. (1E, 1F, 1G and 1H) UMAPs displaying the expression of Prx1/Prrx1, Ctsk, Gli1, and Axin2 in calvarial sutures of 4 day old (1E), 2 month old (1F), 4 month old (1G), and 14 months old (1H) mice.

FIG. 2A-2N. Sub-cluster analysis of the osteogenic cells of the expanding sutures of 4 day old mice. (2A) UMAP plot showing the identity of the subclusters identified among the osteogenic lineage cells. (2B-2E) UMAP plots showing the location of cells expressing Prx1/Prrx1 (2B), Ctsk (2C), Gli1 (2D), and Axin2 (2E). (2F-2G) Unbiased trajectory pseudotime analysis of the cells of the osteogenic lineage, from the most undifferentiated (yellow) to the most differentiated (pink). (2H-2N) Trajectory analysis identifying Prx1/Prrx1 expressing cells (2H), Alkaline phosphatase (Alp1) expressing cells (2I), Runx2 (2J), Bone sialoproteins (Ibsp) expressing cells (2K), Osterix (Sp7) expressing cells (2L), Collagen Type 1(Co11a1) expressing cells (2M), and Osteocalcin expressing cells (2N).

FIG. 3A-3J. Expansion of the sagittal suture increases the number of suture resident sSSCs. (3A) Expansion device. An expansion device made of an orthodontic nickel-titanium wire (0.3 mm in diameter), able to deliver an initial 0.2 N of tensile force, is inserted into two equidistant holes created 2 mm from the sagittal suture, in the parietal bones of 2 month old mice. After insertion, the calvarial skin is repositioned over the device and the wound is closed with resorbable sutures. In control mice of the same age, holes are created but the expansion device is not inserted (mock surgery). (3B, 3C) Suture isolation. 7 days post-surgery, non-expanded (3B) and expanded (3C) calvarial sutures are dissected and cells are isolated. (3D-3E) Histological evaluation (3D) non-expanded and (3E) expanded. 7 days post-surgery, coronal (frontal) sections of the sagittal suture show that the applied tensile force increased the sagittal suture width (black arrows indicate the newly formed tissue between the two osteogenic fronts). (3F-3G)) scRNA-seq analysis. Uniform Manifold Approximation and Projection (UMAP) plot showing unbiased graph-based clusters distribution of all cell populations in the non-expanded (3F) and expanded (3G) sutures isolated from 2 month old animals (6 mice/group). (3H) IVM evaluated areas. IVM is utilized to image cells expressing EGFP (co-expressing Prx1/Prrx1) in three equidistantly distributed regions (A, B, and C) along the total length of the sagittal suture of Prx1-creER-EGFP 2 month old mice. (3I) IVM visualization of EGFP-expressing cells 7 days post-surgery. Left: IVM (maximum intensity projection) of the sagittal suture in non-expanded control animals (coronal view). Right: IVM maximum intensity projection of the sagittal suture in expanded animals (superior view and coronal view). Dashed lines demarcate the sagittal suture space (S.S.)(note the different scale bars in the non-expanded and expanded images). Left parietal (L.P.) and right parietal (R.P.) bone is visualized by second harmonic generation (blue). PRX1+ cells are visualized by expression of EGFP (green). (3J) IVM quantification. 7 days post-surgery, the number of cells expressing EGFP (co-expressing Prx1/Prrx1) are quantified in control and expanded sutures (n=4-5, averages of total cells counted in regions A, B, and C)(**p<0.01).

FIG. 4A-4P. Expansion of the sagittal suture induces expression of Birc5, Ccnd1, Esp11, or Ki67. (4A-4D) scRNA-seq quantification of the expression of Birc5 (4A), Ccnd1 (4B), Esp11 (4C), Ki67 (4D) in Prx1/Prrx1 expressing cells of non-expanded and expanded sutures. (4E-4H) scRNA-seq quantification of the expression of Birc5 (4E), Ccnd1 (4F), Esp11 (4G), Ki67 (4H) in Runx2 expressing cells of non-expanded and expanded sutures. (4I-4L) scRNA-seq quantification of the expression of Birc5 (4I), Ccnd1 (4J), Esp11 (4K), Ki67 (4L) in Sp7 expressing cells of non-expanded and expanded sutures. (4M-4P) scRNA-seq quantification of the expression of Birc5 (4M), Ccnd1 (4N), Esp11 (4O), Ki67 (4P) in Osteocalcin expressing cells of non-expanded and expanded sutures. Dots represent single cells and numerical values on the Y axes indicate the level of expression of the Birc5, Ccnd1, Esp11, or Ki67 gene. * p<0.05 (6 mice/group).

FIG. 5A-5K. Sub-cluster analysis of the osteogenic cells of the mechanically expanded sutures. (5A) UMAP plot showing the identity of the subclusters identified among the osteogenic lineage cells. (5B-5E) UMAP plots showing the location of cells expressing Prx1/Prrx1 (5B), Ctsk (5C), Gli1 (5D), and Axin2 (5E). (5F-5G) Unbiased trajectory pseudotime analysis of the cells of the osteogenic lineage, from the most undifferentiated (yellow) to the most differentiated (pink). (5H-5K) Trajectory analysis identifying Prx1/Prrx1 expressing cells (5H), Bone sialoproteins (Ibsp) expressing cells (5I), Collagen Type 1(Co11a1) expressing cells (5J), and Osteocalcin expressing cells (5K).

FIG. 6A-6B. Sub-cluster analyses of the osteogenic cells of the mechanically expanded and the naturally expanding sutures. (6A) UMAP plot showing the identity of the subclusters identified among the osteogenic lineage cells of both samples. (6B) UMAP plots showing the location of cells of the mechanically expanded sutures (EXPANDED, in red) and of the naturally expanding sutures (4 day old, in blue).

FIG. 7A-7D. Expansion of the sagittal suture enhances the regeneration of a e-CSD remotely located from the suture. 7A) μCT rendering (left, whole skull superior view, with cranial base bones visible through the defect; right, coronal (frontal) section of the parietal bones) and histological sections of the sagittal suture (depicted by green dashed line) and of the defect (depicted by blue dashed line) in non-expanded 2 month old PRX1-creER-EGFP^{+/-} mice. 7B) μCT rendering (left, whole skull superior view; right, coronal (frontal) section of the parietal bones) and histological sections of the sagittal suture (depicted by green dashed line) and of the defect (depicted by blue dashed line) in expanded 2 month old PRX1-creER-EGFP^{+/-} mice. Samples were obtained 60 days after creation of the defects. Red arrows indicate the e-CSD, located 3 mm lateral to the sagittal suture. Blue arrows indicate the sagittal sinus. The implanted radiopaque expansion devise can be seen in the coronal view. H&E and Goldner's Trichrome were used for staining of unpermineralized tissue sections. (7C and 7D) μCT quantification of bone volume (BV) (7C) and of the ratio between bone volume (BV) total volume (TV) (7D) regenerated within the e-CSDs, 60 days after surgery (n=4-5, **p<0.01).

FIG. 8A-8F. Regeneration of e-CSDs sustained by mechanical expansion of the sagittal suture is mediated by Wnt signaling. (8A) μCT rendering (whole skull superior view and coronal (frontal) section of the parietal bones) and histological sections of the sagittal suture (depicted by green dashed line) and of the e-CSD (depicted by blue dashed line) in suture expanded 2 month old Prx1-creER-EGFP^{+/-}; β-catenin^{+/+} mice 60 days after creation of the defect. (8B) μCT rendering (whole skull superior view with cranial base bones visible through the defect, and coronal (frontal) section of the parietal bones) and histological sections of the sagittal suture (depicted by green dashed line) and of the e-CSD (depicted by blue dashed line) in suture expanded 8 week old Prx1-creER-EGFP^{+/-}; β-catenin^{-/-} mice 60 days after creation of the defect. Red arrows indicate the e-CSD, located 3 mm lateral to the sagittal suture. Blue arrows indicate the sagittal sinus. (8C) μCT quantification of the regenerated Bone Volume (BV) in e-CSDs 60 days after surgery (n=5). (8D) μCT quantification of regenerated Bone Volume (BV)/Total Volume (TV) in e-CSDs 60 days after surgery (n=5). (8E-8F) qPCR of canonical Wnt signaling responsive genes (Axin2) (8E) and (β-catenin (Ctnnb1) (8F) in Prx1/Prrx1 expressing cells isolated from PRX1-creER-EGFP^{+/-}; β-catenin^{+/+} and PRX1-creER-EGFP^{+/-}; β-catenin^{-/-} mice treated with tamoxifen (6 pooled mice/group, two technical replicates/group). All mice were treated with tamoxifen for 10 days, starting 5 days before surgery. *p<0.05, **p<0.01.

FIG. 9A-9D. Prx1/Prrx1 is expressed in human calvarial sutures. (9A-9C) In situ hybridization in the sagittal suture of human fetal calvaria (Day 80 post-conception). (9A) Negative control targeting dapB bacterial gene (dapB-AF647) demonstrate no detectable signal. (9B) Positive control targeting polyubiquitin-C(UBC-AF647) is detected in most of the cells. (9C) Sections stained with the probe targeting human Prx1 (hPrrx1-AF647) present with signal in a discrete number of suture cells (see 2X inset). Dashed lines (left) identify calvarial bones. (9D) Quantitative RT-PCR analysis of gene expression in primary cells isolated from the parietal bone and from the sagittal suture of human fetal calvaria. Quantitative PCR was performed to assess the expression of Prx1/Prrx1 in primary cells derived from the parietal bone (P Bone) and from the sagittal suture (Sag Sut). Post conception age and sex of the fetus are shown in parentheses.

FIG. 10. Prx1/Prrx1 is highly expressed in Progenitor Cells of the calvarial sutures of 4 day oldmice. Dot plot scRNA-seq quantification of Prx1/Prrx1 in Progenitor cells (PC), Proliferative osteogenic cells (PRO), Osteoblast precursors (OP), Mature osteoblasts (MO), and Osteocytes (OC). Statistical analysis was performed using the Hurdle model, with *** indicating p<0.001. FC=fold change. Dots represent single cells and numerical values on the Y axes indicate the level of gene expression. (6 mice/group).

FIGS. 11A-11C Ctsk, Gli1, and Axin2 are highly expressed in Progenitor Cells of the calvarial sutures of 4 day old mice. Dot plot scRNA-seq quantification of Ctsk (11A), Gli1 (11B), and Axin2 (11C) in Progenitor cells (PC), Proliferative osteogenic cells (PRO), Osteoblast precursors (OP), Mature osteoblasts (MO), and Osteocytes (OC). Statistical analysis was performed using the Hurdle model, with *** indicating p<0.001. ** indicating p<0.01. and * indicating p<0.05. FC=fold change. Dots represent single cells and numerical values on the Y axes indicate the level of gene expression. (6 mice/group).

FIGS. 12A-12D Trajectory analysis with pseudotime of the osteogenic subclusters of the sutures of 4 day old mice (12A), identifying Ctsk (12B), Gli1 (12C), and Axin2 (12D) expressing cells.

FIGS. 13A-13B Wnt signaling is inhibited in progenitor cells of 4 day old calvarial sutures. (13A) scRNA-seq quantification of β-catenin (Ctnnb1) in Progenitor cells (PC), Proliferative osteogenic cells (PRO), Osteoblast precursors (OP), Mature osteoblasts (MO), and Osteocytes (OC). (13B) scRNA-seq quantification of Tcf7 in Progenitor cells (PC), Proliferative osteogenic cells (PRO), Osteoblast precursors (OP), Mature osteoblasts (MO), and Osteocytes (OC). Statistical analysis was performed using the Hurdle model, with *** indicating p<0.001. ** indicating p<0.01. and * indicating p<0.05. FC=fold change. Dots represent single cells and numerical values on the Y axes indicate the level of gene expression. (6 mice/group).

FIGS. 14A-14P Gene expression of Birc5, Ccnd1, Esp11, or Ki67 in Prx1/Prrx1, Ctsk, Gli1, and Axin2 expressing cells of 2 month old and 4 day old mice. (14A-14D)) scRNA-seq quantification of the expression of Birc5 (14A), Ccnd1 (14B), Esp11 (14C), Ki67 (14D) in Prx1/Prrx1 expressing cells. (14E-14H) scRNA-seq quantification of the expression of Birc5 (14E), Ccnd1 (14F), Esp11 (14G), Ki67 (14H) in Ctsk expressing cells. (14I-14L) scRNA-seq quantification of the expression of Birc5 (14I), Ccnd1 (14J), Esp11 (14K), Ki67 (14L) in Gli1 expressing cells. (14M-14P) scRNA-seq quantification of the expression of Birc5 (14M), Ccnd1 (14N), Esp11 (14O), Ki67 (14P) inAxin2 expressing cells. Statistical analysis was performed using the Hurdle model, with * indicating p<0.05. FC=fold change. Dots represent single cells and numerical values on the Y axes indicate the level of gene expression.

FIGS. 15A-15L Expansion of the sagittal suture induces expression of Birc5, Ccnd1, Esp11, or Ki67 in Ctsk, Gli1, and Axin2 expressing cells. (15A-15D) scRNA-seq quantification of the expression of Birc5 (15A), Ccnd1 (15B), Esp11 (15C), Ki67 (15D) in Ctsk expressing cells. (15E-15H) scRNA-seq quantification of the expression of Birc5 (15E), Ccnd1 (15F), Esp11 (15G), Ki67 (15H) in Gli1 expressing cells. (15I-15L) scRNA-seq quantification of the expression of Birc5 (151), Ccnd1 (15J), Esp11 (15K), Ki67 (15L) in Axin2 expressing cells. Statistical analysis was performed using the Hurdle model, with * indicating p<0.05. FC=fold change. Dots represent single cells and numerical values on the Y axes indicate the level of gene expression.

FIGS. 16A-16F The expansion of the sagittal suture induces proliferation of suture resident Prx1/Prrx1 expressing cells. (16A-16D) qPCR analysis of the expression of Birc5 (16A), Ccnd1 (16C), Esp11 (16B), Ki67 (16D) in Prx1/Prrx1 expressing cells of non-expanded and expanded sutures of 2 months old Prx1-creER-EGFP mice (2 days after surgery). Two days after surgery, relative expression of Birc5, Ccnd1, Esp11, and Ki67 was quantified in EGFP expressing cells (co-expressing Prx1/Prrx1). The quantitative analysis shows significantly higher levels of all 4 genes in Prx1/Prrx1 expressing cells isolated from the expanded sutures (2 independent experiments performed, n=4 and n=6 for each experiment) (*p<0.05, **p<0.01). (16E-16F) In situ hybridization of Ki67 and EGFP mRNA expression, in non-expanded (16E) and in expanded (16F) sagittal sutures of 2 month old Prx1-creER-EGFP+/− mice (2 days after surgery). Top: In coronal (frontal) sections of non-expanded sutures, isolated expression of Ki67 mRNA and EGFP mRNA is observed (red dots and green dots respectively, indicated by red and green arrows). Bottom: In coronal sections of expanded sutures (with limited view due to the expansion), co-localization of Ki67 and EGFP mRNAs is observed (red dots and green dots, co-localized within the same cells, indicated by yellow arrows). DAPI staining of nuclei is in blue. Dashed lines identify suture margins.

FIGS. 17A-17E Single cell RNA sequencing analysis of the mechanically expanded sutures of 2 month old mice. (17A) Uniform Manifold Approximation and Projection (UMAP) plot showing unbiased graph-based clusters distribution of all cell populations of the sutures, isolated after expansion. (17B-17E) UMAPs displaying the expression of Prx1/Prrx1 (17B), Ctsk (17C), Gli1 (17D), and Axin2 (17E). Due to their low number, cells expressing Gli1 and Axin2, although located almost exclusively in the osteogenic cluster, are not well depictable (blue arrows).

FIGS. 18A-18B Surgical steps for insertion of the expansion devise and creation on the c-CSD. (18A) Green arrow points at expansion devise, red arrow points at e-CSD created at a distance of 3 mm from the sagittal suture and 1 mm from the lambdoid suture. (18B) after insertion, the wound is sutured to fully cover the expansion devise.

FIGS. 19A-19B Visualization of the progeny of the cSSCs expressing Prx1/Prrx1 within the c-CSD, 60 days post-surgery. CT rendering and IVM image of a e-CSD, 60 days after creation of the defect (19A) Defect healed in non-expanded 8 week-old Prx1-creER-EGFP+/−;tdTOMATO+/− mice. (19B) Defect healed in expanded 8 week-old Prx1-creER-EGFP+/−;tdTOMATO+/− mice. To induce expression of tdTOMATO, mice were injected with tamoxifen (intraperitoneally, 40 mg/kg in sterile oil) 5 days before and 5 days after surgery. IVM of the regenerated tissue was performed 60 days after defect creation. The e-CSD is depicted by the yellow dashed circle and the area of the defect visualized by IVM (maximum intensity projection) is identified by the red rectangle. Bone is visualized by second harmonic generation (blue) and tdTOMATO expressing cells representing the progeny of the cSSCs expressing Prx1/Prrx1 are shown in red. The implanted radiopaque expansion devise can be seen in the oblique/side view. (n=2).

FIGS. 20A-20D Regeneration of e-CSDs sustained by mechanical expansion of the sagittal suture in 10 month old mice. (20A) CT rendering (whole skull superior view and coronal (frontal) section of parietal bones) in non-expanded 4 month old Prx1-creER-EGFP+/− mice 60 days after creation of the defect. (20B) CT rendering (whole skull superior view and coronal (frontal) section of parietal bones) in expanded 4 month old Prx1-creER-EGFP+/− mice 60 days after creation of the defect. (20C) μCT quantification of the regenerated Bone Volume (BV) in e-CSDs 60 days after surgery. (20D) CT quantification of regenerated Bone Volume (BV)/Total Volume (TV) in e-CSDs 60 days after surgery. n=5, with e-CSD created at 1 mm from the sagittal and the lambdoid sutures.

FIGS. 21A-21M. Mechanical expansion of the sagittal suture induces proliferation of PRX1 expressing SSCs and sustains endogenous regeneration of a remotely created parietal bone critical size defect (CSD). (21A) ScRNA-seq analysis (UMAP) of the cells of the calvarial sutures of 4-day old mice (n=6). PRX1 is highly expressed in the osteogenic cell clusters. (21B) ScRNA-seq analysis (UMAP) of the cells of the calvarial sutures of 4-month old mice (n=6). PRX1 expression is no longer detectable in skeletally mature mice. (21C) Expansion devise: An expansion device made on an orthodontic nickel-titanium wire, able to deliver an initial 0.2 N of tensile force, is inserted into two equidistant holes created 2 mm from the sagittal suture, in the parietal bones of 8 week old PRX1-creER-EGFP+/+ male mice. In control mice only the holes are created, and the expansion device is not applied. (21D) Evaluated areas: Intravital microscopy (IVM) is utilized to image PRX1 expressing cells (EGFP+ cells) in three equidistant areas along the sagittal suture (named A, B, and C areas). (21E) Quantification of PRX1 expressing cells: 7 days post-surgery, EGFP+ are quantified in control and expanded sutures (n=4-5, averages of total cells in A, B, and C areas). See Wilk et al., 2017 for quantification methodology (**p<0.01). (21F) Example of intravital microscopy (IVM) evaluation (7 days post expansion) utilized for the quantification: LEFT: sagittal suture in control mice. RIGHT: expanded sagittal suture in test animals. (21G) In situ hybridization (2 days post expansion): RNAscope® in situ hybridization (Advanced Cell Diagnostics, Inc., USA) shows that during suture expansion EGFP+ cells co-express Ki67 (a gene expressed during cell proliferation)(green arrows indicate co-localized EGFP and Ki67 signals, yellow arrows indicate Ki67 signal alone)(dashed lines identify suture margins). (21H-21J) qRT-PCR in PRX1 expressing cells: Expression of proliferation markers (DKK1 (21J), Ki67 (21H), and ESPL1 (21I)). (Ki67 (21H) and ESPL1 (21I)) is upregulated in PRX1 expressing cells isolated by FACS from expanded sutures, 2 days after the expansion is initiated (4 animals/group)(**p<0.01). (21K) CT & Histological Evaluations: A CSD of 2 mm in diameter, created in the parietal bone of 8-week old mice, 3 mm from the sagittal suture, simultaneously to the insertion of the expanding device, fully regenerates after 60 days. In control mice (mock surgery with no expansion device inserted) full regeneration of the CSD is never observed (red arrows indicate the CSD, green arrows indicate the sagittal suture; black arrow indicate the expansion device; green-framed inset shows frontal sections of sagittal sutures). (21L-21M) Lineage Tracing Analysis (60 days post-surgery —defect site): 8-week old PRX1-creER-EGFP+/−;tdTOMATO+/− male mice underwent the same surgical treatments described above. After surgery, for five consecutive days mice were treated with tamoxifen (40 mg/Kg of body weight, IP) and 60 days after surgery CSDs were analyzed with IVM to detect tdTOMATO signal in progeny of PRX1 expressing cells within the bone defects (tdTOMATO expressing cells are show in red by maximum-intensity projection over 200 μm depth, bone is depicted in blue by means of second harmonic generation, and dashed lines outline the defect's original margin; n=2)((21L), CSD in control mice; (21M), CSD defect in mice with expanded sutures).

FIGS. 22A-22H. Niclosamide induces proliferation of PRX1 expressing SSCs in vitro and ex vivo. (22A-22B) MTT proliferation assays of primary PRX1 expressing cells isolated from the calvarial sutures of PRX1-creER-EGFP+/+ mice. After 24 hours (22A) and 48 hours (22B), Niclosamide and Pyrvinium induce a statistically significant proliferation. (22C) MTT proliferation assays of immortalized PRX1 expressing cells isolated from the calvarial sutures of PRX1-creER-EGFP+/+ mice. After 48 hours, Niclosamide induces a statistically significant proliferation. (22D-22F) Quantitative RT-PCR of osteoblast differentiation genes of immortalized PRX1 expressing cells isolated from the calvarial sutures of PRX1-creER-EGFP+/+ mice. After 48 hours, Niclosamide induces a statistically significant downregulation of the expression of RUNX2 (22D), SP7 (22E), and Osteocalcin (OCN) (22F). (22G-22H) FAC-sorting analysis of tissue-cultured sutures isolated from of PRX1-creER-EGFP+/+ mice. Suture were explanted and cultured ex vivo. After 2 weeks of incubation with Niclosamide, a statistically significant increase of the GFP+ cells (22G) (PRX1 expressing cells) is observed.

However, no increase of GFP-cells (22H) is detected. All above studies (22A-22E) were repeated 2 or 3 times, with consistent results.

FIGS. 23A-23D. Niclosamide induces proliferation of PRX1 expressing SSCs in vivo without altering the bone mass of long bones. (23A-23C) Flow cytometry was utilized to quantify the number of PRX1 expressing SSCs after treatment with Niclosamide (20 mg/kg of body weight, IP, 5 days/week, for 3 consecutive weeks) or with vehicle (control) in calvarial sutures (23A), in the periosteum of long bones (23B), and in the periodontal ligament of posterior molar teeth (23C). 4 mice/treatment (2 males and 2 females). (23D) Micro-computed tomography was utilized to measure bone mass (BV/TV) in the femurs of male and female mice after treatment with Niclosamide (20 mg/kg of body weight, IP, 5 days/week for 3 consecutive weeks) or with vehicle (control) (n=2).

FIGS. 24A-24B. shows images of a critical size calvarialbone defect (2 mm in diameter) in the right parietal bone of skeletally mature C57Bl/6 female mice (8 week old mice). Immediately after creation of the defect, 10 μL of a 12% carboxymethylcellulose gel (CONTROL) (24A) or 10 μL of a 12% carboxymethylcellulose gel containing 100 μg of niclosamide (TEST) (24B) were implanted within the bone defect.

FIG. 25. shows a graph of bone regeneration assessed by means of μCT, 4 weeks after surgery (FIG. 24). Student's t-test was used for statistical evaluation (n=3, ** indicates p<0.01).

FIGS. 26A-26E quantitative analysis of the number of cells expressing Prx1 (and co-expressing GFP) was performed by means of flow cytometry (26A-26D) for various concentrations (DMSO control (26A), 0.02 μM (26B), 0.2 μM (26C), and 2 μM (26D)) for 48 hours. (26E) is a graph showing the results of the quantitative analysis of the number of cells expressing Prx1 and co-expressing GFP.

FIGS. 27A-27D show bone regeneration was assessed by means of μCT, 8 weeks after surgery (27A-27C) 3D rendering, (27D) quantitative assessment of the regenerated Bone Volume (BV) within the Total Volume (TV) of the calvarial bone defects.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

As used herein, “Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n— COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

As used herein, “Paired related homeobox 1 (PRX1)” (or “PRRX1”) is transcription factor highly expressed during limb and craniofacial development, and postnatally in various stem cells of the body. The amino acid sequence of PRX1 and DNA and RNA sequences encoding it are well known in the art. In one embodiment, the PRX1 comprises the amino acid sequence shown for NCBI Reference Sequence: NP_008833, NP 073207, NP_001020741,

SEQ ID NO: 1
(Human PRX1_Paired mesoderm homeobox protein 1_UniProtKB/
Swiss-Prot P54821-1_NCBI Reference Sequence: NP_008833)
MTSSYGHVLERQPALGGRLDSPGNLDTLQAKKNFSVSHLLDLEEAGDMVAAQA
DENVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYSAMATYSATCANNSPAQGINMANSIANLR
LKAKEYSLQRNQVPTVN
SEQ ID NO: 2
(Human PRX1_Paired mesoderm homeobox protein 1_UniProtKB/
Swiss-Prot P54821-2_NCBI Reference Sequence: NP_073207)
MTSSYGHVLERQPALGGRLDSPGNLDTLQAKKNFSVSHLLDLEEAGDMVAAQA
DENVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYRSSSLPRCCLHEGLHNGF
SEQ ID NO: 3
(Mouse PRX1_Paired related homeobox protein 1_UniProtKB/Swiss-
Prot G3UZ44_NCBI Reference Sequence: NP_001020741)
MTSSYGHVLERQPALGGRLDSPGNLDTLQAKKNFSVSHLLDLEEAGDMVAAQA
DESVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYR
SEQ ID NO: 4
(Mouse PRX1_Paired mesoderm homeobox protein 1_UniProtKB/
Swiss-Prot P63013-1_NCBI Reference Sequence: NP_035257)
MTSSYGHVLERQPALGGRLDSPGNLDTLQAKKNFSVSHLLDLEEAGDMVAAQA
DESVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYSAMATYSATCANNSPAQGINMANSIANLR
LKAKEYSLQRNQVPTVN
SEQ ID NO: 4
(Mouse PRX1_Paired mesoderm homeobox protein 1_UniProtKB/
Swiss-Prot P63013-2_NCBI Reference Sequence: NP_035257)
MTSSYGHVLERQPALGGRLDSPGNLDTLQAKKNFSVSHLLDLEEAGDMVAAQA
DESVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYRSSSLPRCCLHEGLHNGF
SEQ ID NO: 5
(Mouse PRX1_Paired mesoderm homeobox protein 1_UniProtKB/
Swiss-Prot P43271_NCBI Reference Sequence: NP_035257)
MTSSYGHVLERQPALGGRLDSPGNLDTLQAKKNFSVSHLLDLEEAGDMVAAQA
DESVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYSAMATYSATCANNSPAQGINMANSIANLR
LKAKEYSLQRNQVPTVN
SEQ ID NO: 6
(Mouse PRX1_Paired mesoderm homeobox protein 1_UniProtKB/
Swiss-Prot Q02810_NCBI Reference Sequence: NP_035257)
MTSSYGHVLERQPALGGRLDSPGNLDTLQAKKNFSVSHLLDLEEAGDMVAAQA
DESVGEAGRSLLESPGLTSGSDTPQQDNDQLNSEEKKKRKQRRNRTTFNSSQLQALERV
FERTHYPDAFVREDLARRVNLTEARVQVWFQNRRAKFRRNERAMLANKNASLLKSYS
GDVTAVEQPIVPRPAPRPTDYLSWGTASPYRSSSLPRCCLHEGLHNGF

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. Ph in Formula I refers to a phenyl group.

The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₁, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl−1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₅, C₂-C₁₆, C₂-C₁₄, C₂-C₁₂, C₂-C₁₀, C₂-C₅, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl−1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₅, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₅, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O—.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula ZOZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, -NRaSO₂Rb, —C(═O)Ra, —C(═O)ORa, —C(═O)NRaRb, —OC(═O)NRaRb, —ORa, —SRa, —SORa, -S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

### Compositions

Described herein are pharmaceutical compositions comprising an effective amount of niclosamide, or a pharmaceutically acceptable salt, prodrug, or derivative thereof; and a pharmaceutically acceptable carrier. In some embodiments, the compositions can further include an effective amount of pyrvinium, or a pharmaceutically acceptable salt, prodrug, or derivative thereof; quercetin, or a pharmaceutically acceptable salt, prodrug, or derivative thereof; or any combination thereof.

In some embodiments, the pharmaceutical composition can include an effective amount of niclosamide, or a pharmaceutically acceptable salt, prodrug, or derivative thereof; pyrvinium, or a pharmaceutically acceptable salt, prodrug, or derivative thereof; quercetin, or a pharmaceutically acceptable salt, prodrug, or derivative thereof; and a pharmaceutically acceptable carrier and a pharmaceutically acceptable carrier.

In some embodiments, the pharmaceutical composition can include an effective amount of niclosamide, or a pharmaceutically acceptable salt, prodrug, or derivative thereof; quercetin, or a pharmaceutically acceptable salt, prodrug, or derivative thereof; and a pharmaceutically acceptable carrier and a pharmaceutically acceptable carrier.

In some embodiments, the pharmaceutical composition can include an effective amount of niclosamide, or a pharmaceutically acceptable salt, prodrug, or derivative thereof; pyrvinium, or a pharmaceutically acceptable salt, prodrug, or derivative thereof; and a pharmaceutically acceptable carrier and a pharmaceutically acceptable carrier.

### Niclosamide

Niclosamide is a compound of formula I:

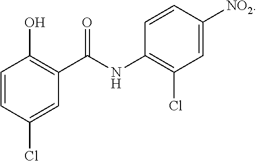 [SMILES: O=C(Nc1ccc([N+](=O)[O-])cc1Cl)c1cc(Cl)ccc1O]

As referenced here niclosamide derivatives/analogs are disclosed generically, sub generically and specifically in any one or more of International Patent Publication No. WO 2004/006906; U.S. Patent Publication Nos. US20100041657A1; US 2009/0062396; US20130231312; U.S. Pat. Nos. 7,132,546; 7,989,498; 8,263,857; 10,905,666; and 11,331,290, each of which is incorporated herein by reference in its entirety.

### Pyrvinium

Pyrvinium is a compound of formula II

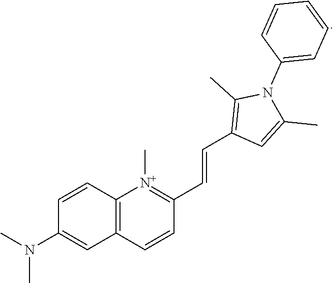 [SMILES: Cc1cc(/C=C/c2ccc3cc(N(C)C)ccc3[n+]2C)c(C)n1-c1ccccc1]

### Quercetin

Quercetin is a compound of formula III

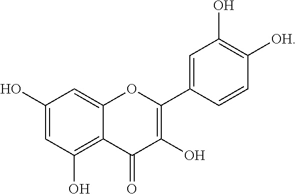 [SMILES: O=c1c(O)c(-c2ccc(O)c(O)c2)oc2cc(O)cc(O)c12]

In some embodiments, niclosamide, or a pharmaceutically acceptable salt, prodrug, or derivative thereof can be present in the composition in an effective amount to induce proliferation of PRX1 expressing stem cells. In some embodiments, the PRX1 expressing stem cells can be selected from skeletal stem cells (SSCs), pulp stem cells, periodontal stem cells, ligament stem cells, adipose stem cells, and dermis stem cells.

In some embodiments, niclosamide, or a pharmaceutically acceptable salt, prodrug, or derivative thereof can be present in the composition in an effective amount to induce proliferation of PRX1 expressing skeletal stem cells (SSCs).

In some embodiments, niclosamide, or a pharmaceutically acceptable salt, prodrug, or derivative thereof can be present in the composition in an effective amount to treat bone diseases, bone fractures, bone injuries, or bone abnormalities. In some embodiments, niclosamide, or a pharmaceutically acceptable salt, prodrug, or derivative thereof can be present in the composition in an effective amount to rejuvenate bone. In some embodiments, niclosamide, or a pharmaceutically acceptable salt, prodrug, or derivative thereof can be present in the composition in an effective amount to regenerate bone.

The term “pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; tale; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, collagen sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, tale, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of the active agent or a pharmaceutically acceptable salt or derivative thereof.

In some embodiments, the carrier can include a collagen sponge. In some embodiments, the carrier can include a carboxymethylcellulose.

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. The active agent may be administered by any route. In some embodiments, the active ingredient is administered via a variety of routes, including oral, intravenous, intramuscular, intrauterine, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc. Administration of the active components of their compositions can be a single administration, or at continuous and distinct intervals as can be readily determined by a person skilled in the art.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the compositions can be administered at an early stage in the healing of a bone fracture or bone injury. In some embodiments, the compositions can be administered prior to performing a bone-related surgical procedure (orthopedic surgery) on a subject. Bone-related surgical procedures can include, but are not limited to, ACL and meniscus repair, hip replacement, spinal fusion, knee replacement, shoulder arthroscopy and debridement, fractures repair, rotator cuff repair, carpel tunnel release, intervertebral disk surgery, cranial surgery, removal of support implant, or any combination thereof.

### Methods of Use

Described herein are methods of inducing proliferation of PRX1 expressing stem cells in a subject in need thereof, the method comprising in a subject in need thereof, the method comprising administering an effective amount of niclosamide, or a pharmaceutically acceptable salt, prodrug, or derivative thereof; or a pharmaceutical composition described herein.

In some embodiments, the PRX1 expressing stem cells can include, but are not limited to, skeletal stem cells, pulp stem cells, periodontal stem cells, ligament stem cells, adipose stem cells, and dermis stem cells. In some embodiments, the PRX1 expressing stem cells can include skeletal stem cells. In some embodiments, the PRX1 expressing stem cells can include pulp stem cells. In some embodiments, the PRX1 expressing stem cells can include periodontal stem cells. In some embodiments, the PRX1 expressing stem cells can include ligament stem cells. In some embodiments, the PRX1 expressing stem cells can include adipose stem cells. In some embodiments, the PRX1 expressing stem cells can include dermis stem cells.

In some embodiments, the PRX1 expressing stem cells can include a PRX1 amino acid sequence including SEQ ID NO: 1, SEQ ID NO: 2, SEQ ID NO: 3, SEQ ID NO: 4, SEQ ID NO: 5, or SEQ ID NO: 6.

Described herein are methods of inducing proliferation of skeletal stem cells (SSCs) in a subject in need thereof, the method comprising in a subject in need thereof, the method comprising administering an effective amount of niclosamide, or a pharmaceutically acceptable salt, prodrug, or derivative thereof; or a pharmaceutical composition described herein.

Described herein are also methods for bone rejuvenation and/or bone regeneration in a subject in need thereof, the method comprising administering an effective amount of niclosamide, or a pharmaceutically acceptable salt, prodrug, or derivative thereof; or a composition described herein.

Described herein are also methods for treating a bone-related disease, bone fracture, bone injury, or bone abnormality (e.g., bone developmental abnormality), the method comprising administering an effective amount of niclosamide, or a pharmaceutically acceptable salt, prodrug, or derivative thereof; or a composition described herein.

In some embodiments, the methods can treat a bone-related disease, bone fracture, bone injury, or bone abnormality (e.g., bone developmental abnormality). In some embodiments, the methods can treat a bone-related disease. In some embodiments, the methods can treat a bone fracture. In some embodiments, the methods can treat a bone injury. In some embodiments, the methods can treat a bone abnormality (e.g., bone developmental abnormality). In some embodiments, the method can rejuvenate bone. In some embodiments, the method can regenerate bone. In some embodiments, bone types can include but are not limited to, skull bones (e.g., jaw bone), spine bones (e.g., cervical, thoracic and lumbar vertebrae, sacrum, tailbone (coccyx)), chest bones (e.g., ribs and breastbone (sternum)), arm bones (e.g., shoulder blade (scapula), collar bone (clavicle), humerus, radius, ulna), hand bones (e.g, wrist bones (carpals), metacarpals and phalanges), pelvis bones (e.g., hip bones), leg bones (e.g, thigh bone (femur), kneecap (patella), shin bone (tibia),fibula), and/or feet bones (e.g., tarsals, metatarsals, phalanges). In some embodiments, the methods for bone regeneration can include periodontal bone regeneration.

In some embodiments, the bone related disease comprises osteoporosis, osteopenia, osteomalacia, rheumatoid arthritis, tendonitis, osteoarthritis, gouty arthritis, alveolar bone loss, osteotomy bone loss, systemic mastocytosis, adult hypophosphatasia, hyperadrenocorticism, osteogenesis imperfecta, Paget's disease, craniosynostosis, skeletal dysplasias, osteochondrodysplasias, osteogenesis imperfecta, Cushing's disease/syndrome, Tumer syndrome, Gaucher disease, Ehlers-Danlos syndrome, Marfan's syndrome, Menkes' syndrome, Fanconi's syndrome, hypercalcemia, hypocalcemia, arthritides, periodontal bone related disease, childhood idiopathic bone loss, Paget's disease, scoliosis bone loss due to metastatic cancer, osteolytic lesions, rickets (including vitamin D dependent, type I and II, and x-linked hypophosphatemic rickets), fibrogenesis imperfecta ossium, osteosclerotic disorders such as pycnodysostosis and damage caused by macrophage-mediated inflammatory processes, or any combination thereof. In some embodiments, the bone related disease can be periodontal bone related disease. In some embodiments, the bone related disease can be osteoporosis. In some embodiments, the bone relate disease can be osteoarthritis. In some embodiments, the bone related disease can be craniosynostosis. In some embodiments, when the bone relate disease is a bone developmental abnormality (e.g. craniosynostosis) the compositions can be administered intrauterine. In some embodiments, when the bone relate disease is a bone developmental abnormality (e.g. craniosynostosis) the compositions can be administered to a newborn prior to suture fusion. In some embodiments, the method induces proliferation of PRX1 expressing stem cells. In some embodiments, the PRX1 expressing stem cells can be skeletal stem cells. In some embodiments, the PRX1 expressing stem cells can be ligament stem cells.

In some embodiments, the method induces proliferation of PRX1 expressing stem cells. In some embodiments, the PRX1 expressing stem cells can include, but are not limited to, skeletal stem cells, pulp stem cells, periodontal stem cells, ligament stem cells, adipose stem cells, and dermis stem cells. In some embodiments, the PRX1 expressing stem cells can be skeletal stem cells. In some embodiments, the PRX1 expressing stem cells can be pulp stem cells. In some embodiments, the PRX1 expressing stem cells can be periodontal stem cells. In some embodiments, the method induces proliferation of skeletal stem cells. In some embodiments, the method induces proliferation of pulp stem cells. In some embodiments, the method induces proliferation of periodontal stem cells. In some embodiments, the method induces proliferation of ligament stem cells. In some embodiments, the method induces proliferation of adipose stem cells. In some embodiments, the method induces proliferation of dermis stem cells.

Described herein are also methods of treating a periodontal bone related disease, the method comprising administering an effective amount of niclosamide, or a pharmaceutically acceptable salt, prodrug, or derivative thereof; or a composition described herein. In some embodiments, the method induces proliferation of PRX1 expressing stem cells. In some embodiments, the PRX1 expressing stem cells can be periodontal stem cells. In some embodiments, the method induces proliferation of periodontal stem cells.

Described herein are also methods of treating a periodontal bone related disease, the method comprising administering an effective amount of niclosamide, or a pharmaceutically acceptable salt, prodrug, or derivative thereof; or a composition described herein. In some embodiments, the method induces proliferation of PRX1 expressing stem cells. In some embodiments, the PRX1 expressing stem cells can be periodontal stem cells. In some embodiments, the method induces proliferation of periodontal stem cells.

Described herein are also methods of treating a skin related disease, the method comprising administering an effective amount of niclosamide, or a pharmaceutically acceptable salt, prodrug, or derivative thereof; or a composition described herein. In some embodiments, the method induces proliferation of PRX1 expressing stem cells. In some embodiments, the PRX1 expressing stem cells can be dermis stem cells.

In some embodiments, the skin related diseases can include, but are not limited to, dermatis, atopic dermatis (e.g., eczema), rosacea, psoriasis, acne, or dermal wounds, or any combination thereof.

In some embodiments, the methods can further include administering an effective amount of pyrvinium, or a pharmaceutically acceptable salt, prodrug, or derivative thereof; quercetin, or a pharmaceutically acceptable salt, prodrug, or derivative thereof; or any combination thereof.

In some embodiments, the methods can include administering an effective amount of niclosamide, or a pharmaceutically acceptable salt, prodrug, or derivative thereof; pyrvinium, or a pharmaceutically acceptable salt, prodrug, or derivative thereof; and quercetin, or a pharmaceutically acceptable salt, prodrug, or derivative thereof; or a pharmaceutical composition including an effective amount of niclosamide, or a pharmaceutically acceptable salt, prodrug, or derivative thereof; pyrvinium, or a pharmaceutically acceptable salt, prodrug, or derivative thereof; quercetin, or a pharmaceutically acceptable salt, prodrug, or derivative thereof, and a pharmaceutically acceptable carrier.

In some embodiments, wherein the methods comprise administering an effective amount of niclosamide, or a pharmaceutically acceptable salt, prodrug, or derivative thereof; and quercetin, or a pharmaceutically acceptable salt, prodrug, or derivative thereof; or a pharmaceutical composition including an effective amount of niclosamide, or a pharmaceutically acceptable salt, prodrug, or derivative thereof; quercetin, or a pharmaceutically acceptable salt, prodrug, or derivative thereof, and a pharmaceutically acceptable carrier.

In some embodiments, wherein the methods comprise administering an effective amount of niclosamide, or a pharmaceutically acceptable salt, prodrug, or derivative thereof; and pyrvinium, or a pharmaceutically acceptable salt, prodrug, or derivative thereof; or a pharmaceutical composition including an effective amount of niclosamide, or a pharmaceutically acceptable salt, prodrug, or derivative thereof; pyrvinium, or a pharmaceutically acceptable salt, prodrug, or derivative thereof, and a pharmaceutically acceptable carrier.

In some embodiments, the skeletal stem cells (SSCs) can be PRX1 expressing skeletal stem cells (SSCs).

In some embodiments, a subject is determined to have or be at risk of developing a bone related disease based on methods known in the art. For example, a subject is determined to have or be at risk of developing a osteoporosis on the presence of one or more risk factors, e.g., gender (increased risk for females); age (increased risk over age 50 for women or age 70 for men); ethnicity (increased risk for Caucasians and Asians); bone structure and body weight (increased risk for those with small bones and thin frames); family history; prior history of broken bones; menopause/hysterectomy; and medications (e.g., glucocorticoid therapy and androgen deprivation therapy increase risk). Additional factors that increase risk include alcohol intake; smoking; low body mass index; poor nutrition; vitamin D deficiency; eating disorders (e.g., anorexia nervosa, bulimia); insufficient exercise; low dietary calcium intake; and frequent falls. Osteoporosis can also be associated with (e.g., increased risk of developing osteoporosis is associated with) conditions including lupus, rheumatoid arthritis; primary/secondary hypogonadism or low testosterone levels in men; celiac disease; inflammatory bowel disease (IBD) (including different forms of IBD, such as Crohn's disease and ulcerative colitis); weight loss surgeries (such as gastric bypass surgery); diabetes; hyperparathyroidism; hyperthyroidism; amenorrhea; leukemia/lymphoma; sickle cell disease; chronic diseases that reduce mobility (such as stroke, Parkinson's disease, and multiple sclerosis (MS); AIDS/HIV; ankylosing spondylitis; blood and bone marrow disorders; breast cancer and hormone therapies for breast cancer; chronic obstructive pulmonary disease (COPD), including emphysema; Cushing's syndrome; depression; female athlete triad (includes loss of menstrual periods, an eating disorder and excessive exercise); gastrectomy; gastrointestinal bypass procedures; kidney disease that is chronic and long lasting; liver disease that is severe, including biliary cirrhosis; malabsorption syndromes, including celiac disease; multiple myeloma; organ transplants; polio and post-polio syndrome; poor diet, including malnutrition; premature menopause; prostate cancer and hormone therapies for prostate cancer; rheumatoid arthritis; scoliosis; spinal cord injuries; stroke; thalassemia; thyrotoxicosis; and weight loss.

In some embodiments, the methods can include diagnosis risk or presence of osteoporosis based on bone mineral density (BMD). A number of methods for determining BMD are known in the art, including DXA (dual-energy X-ray absorptiometry); pDXA (peripheral DXA); SXA (single-energy X-ray absorptiometry); DPA (dual photon absorptiometry); SPA (single photon absorptiometry); QCT (Quantitative Computed Tomography); and QUS (Quantitative Ultrasound). Most use densitometry to measure BMD.

As noted in Table 6 below, osteoporosis is diagnosed when a person's BMD is equal to or more than 2.5 standard deviations below this reference measurement. Osteopenia is diagnosed when the measurement is between 1 and 2.5 standard deviations below the young adult reference measurement.

TABLE 6

| Status | Hip BMD |
|---|---|
| Normal | T-score of −1 or above |
| Osteopenia | T-score lower than −1 and greater than −2.5 |
| Osteoporosis | T-score of −2.5 or lower |
| Severe | T-score of −2.5 or lower, and presence of at least |
| osteoporosis | ne fragility fracture |

In some embodiments, the methods include determining a subject's BMD, and if the subject's BMD indicates that the subject has osteopenia, osteoporosis, or severe osteoporosis, then administering a niclosamide, or a pharmaceutically acceptable salt, prodrug, or derivative thereof; or a composition as described herein to the subject.

The methods can further include monitoring the subject (e.g., by evaluating frequency of fractures, presence of bone lesions, bone density or bone morphology, e.g., using x-ray or other imaging methods) at selected intervals, e.g., a month, three months, six months, or a year after initiation of the treatment, and selected intervals thereafter, e.g., every month, every three months, every six months, or every year thereafter. An increase in bone density, normal bone morphology, or decrease in frequency of fractures or bone lesions, indicates that the treatment is effective.

For example, the methods can further include monitoring the subject by repeating the BMD test at selected intervals, e.g., a month, three months, six months, or a year after initiation of the treatment, and selected intervals thereafter, e.g., every month, every three months, every six months, or every year thereafter. An increase in the T score indicates that the subject's bone density is increasing, e.g., that the treatment is effective.

In some embodiments, described is a method for treating osteoporosis, the method comprising administering an effective amount of niclosamide, or a pharmaceutically acceptable salt, prodrug, or derivative thereof; pyrvinium, or a pharmaceutically acceptable salt, prodrug, or derivative thereof; quercetin, or a pharmaceutically acceptable salt, prodrug, or derivative thereof; or any combination thereof; or a pharmaceutical composition described herein.

In some embodiments, the compositions described here as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, immunomodulatory agents, anticytokine, antiplatelet agents, anti-cancer drugs, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, histrelin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

By way of non-limiting illustration, examples of certain embodiments of the present disclosure are given below.

### Examples

It was shown that at least in mice, expression of PRX1 (or PRRX1), a transcription factor highly expressed during limb and craniofacial development, identifies a population of post-natal SSCs of the calvarial bone. These calvarial SSCs are present within the calvarial sutures, which represent their biological niches. It was also observed the number of PRX1 expressing SSCs declines with age. This evidence is in line with various studies that have reported a decline of stem cells with aging.

Therefore, by inducing a proliferation of the PRX1 expressing SSCs methods that can reverse the aging process and sustain regeneration of skeletal tissues were investigated.

One such method is based on the mechanical stimulation of the SSCs niche within the calvarial bone. The data shows that such stimulation, in the form of a controlled expansion of the calvarial sagittal sutures, induces proliferation of the PRX1 expressing SSCs resident within the suture and can, consequently, sustain endogenous regeneration of the parietal bone without requiring implantation of biomaterials. This result recapitulates the ability of the calvarial bone of young children (up to 2 years old) to regenerate bone and confirms that PRX1 expressing SSCs can be successfully harnessed for bone regeneration. Thus, next it was investigated whether any small molecule could induce proliferation of the PRX1 expressing SSCs.

Niclosamide can induce proliferation of PRX1 expressing SSCs and therefore Niclosamide is an agent that can be used to rejuvenate skeletal tissues and harness SSCs to sustain bone regeneration.

There is a need for compounds that, by inducing proliferation of skeletal stem cells, allow to harness them for therapies that target bone metabolism and bone regeneration.

### Example 1. Expansion of the sagittal suture induces proliferation of skeletal stem cells and sustains endogenous calvarial bone regeneration

In newborn humans, and up to approximately 2 years of age, calvarial bone defects can naturally regenerate. This remarkable regeneration potential is also found in newborn mice and is absent in adult mice. Since previous studies showed that the mouse calvarial sutures are reservoirs of calvarial skeletal stem cells (cSSCs), which are the cells responsible for calvarial bone regeneration, here it was hypothesized that the regenerative potential of the newborn mouse calvaria is due to a significant amount of cSSCs present in the newborn expanding sutures. Thus, it was tested whether such regenerative potential can be reverse engineered in adult mice by artificially inducing an increase of the cSSCs resident within the adult calvarial sutures.

First, the cellular composition of the calvarial sutures in newborn and in older mice were analyzed, up to 14 month old mice, showing that the sutures of the younger mice are enriched in cSSCs. Then, a controlled mechanical expansion of the functionally closed sagittal sutures of adult mice demonstrated that induces a significant increase of the cSSCs. Finally, if a calvarial critical size bone defect is created simultaneously to the mechanical expansion of the sagittal suture, it fully regenerates without the need for additional therapeutic aids. Using a genetic blockade system, further demonstrate that this endogenous regeneration is mediated by the canonical Wnt signaling.

This study shows that controlled mechanical forces can harness the cSSCs and induce calvarial bone regeneration. Similar harnessing strategies may be used to develop effective bone regeneration autotherapies.

This work describes the discovery that an “activation” of a calvarial suture, in the form of a controlled mechanical expansion, can increase the number of calvarial skeletal stem cells (cSSCs) present in the suture and can, consequently, sustain the regeneration of calvarial bone defects that are otherwise unable to heal. Using this strategy bone regeneration occurs without implantations of biomaterials or other osteogenic tissues within the bone defects. Thus, mechanically induced suture expansion could be utilized to harness cSSCs in challenging calvarial bone regeneration procedures. The same strategy could be validated to activate other skeletal stem cell niches of the skeleton and foster regeneration of bone defects of other skeletal segments.

### INTRODUCTION

Despite recent advancements, bone regeneration of craniofacial defects due to trauma, congenital abnormalities, or cancer remains a difficult task and represents a serious health burden (1-3). Available therapeutic aids, including those based on implantable biomaterials and recombinant growth factors, present limitations in terms of efficacy and safety (4-7). New approaches utilizing stem cells have also been tested with limited success (8,9).

In what appears to be a paradigm shift in craniofacial biology (10), it was previously determined that the postnatal mouse calvarial sutures, in addition to acting as compliant connectors between calvarial bones (synarthroses), serve as reservoirs for calvarial skeletal stem cells (cSSCs) that contribute to calvarial bone regeneration (11-14). For instance, in the studies it was shown that cSSCs expressing Prx1/Prrx1, a transcription factor expressed in the mesenchyme during craniofacial and limb development (11,12), are required for regeneration of calvarial bone defects, and that their progeny is responsible for the bone regeneration process (13). Importantly, previous studies also showed that the number of Prx1/Prrx1 expressing cells declines with aging, while the total number of the cells resident within the sutures remains stable after the sutures are fully developed (13).

Other investigators identified cells expressing Gli1 (Zhao et al. (14)) or Axin2 (Maruyama et al. (15)) as cells of the calvarial sutures with stem cell qualities similar to those of the Prx1/Prrx1 expressing cells. More recently, Debnath et al. (16) also proposed expression of Cathepsin K (Ctsk) as another marker of the cSSCs. The identification of the cSSCs and the characterization of their roles in calvarial bone regeneration warrants additional studies aimed at understanding whether they can be harnessed for more effective craniofacial bone regenerative therapies.

Of much interest to bone regenerative studies, it has been described that in children up to 2 years of age, regeneration of calvarial defects can occur naturally and without therapeutic aids (i.e. implantation of biomaterials of osteogenic tissues) (17,18). This exceptional regenerative potential coincides with the existence of calvarial sutures in rapid expansion, and is lost around 2 years of age, when a substantial functional closure of the sutures occurs (19,20). Importantly, a similar age-related regenerative potential is also observed in mice, whose calvarial bone and suture development is analogous to humans. In fact, mice are utilized to study craniofacial development, craniofacial pathologies (i.e. craniosynostosis), and to develop new bone regeneration approaches (20-25). Therefore, here it was posited that the calvarial bone regeneration potential observed in infant humans and mice is due to a significant number of cSSCs present within the expanding sutures. This hypothesis is supported by evidence showing that, at least in mice, the number of cSSCs declines with aging (13) and that a certain critical amount of cSSCs is required for the calvarial regeneration process to occur (13,26). In fact, as shown in previous studies, a reduction of 80% of the Prx1/Prrx1 expressing cells, induced by means of expression of the Diphtheria toxin gene, significantly impairs regeneration of otherwise spontaneously healing calvarial bone defects (13). Moreover, the distance of a calvarial bone defect from the suture greatly influences the regenerating amount of bone, indicating that bone regeneration can only occur if a sufficient number of cSSCs, or a sufficient number of their progeny, can reach the bone defect (26). Therefore, in the present work the hypothesis was tested by artificially inducing an increase of cSSCs within the functionally closed calvarial sutures of skeletally mature 2 month old (8 week old) mice and by testing the effect of the increase on the regeneration of calvarial bone defects otherwise unable to spontaneously regenerate (calvarial critical size defect, hereafter identified as e-CSD).

First, single cell RNA sequencing (scRNA-seq) was employed to compare the cellular composition of the calvarial sutures in mice of different ages, from the rapidly expanding calvarial sutures of 4 day old mice to the fully developed and aged sutures of 14 month old mice. Such evaluation confirmed that a significantly higher number of cSSCs is present in the developing open sutures of the 4 day old mice. Then, utilizing scRNAseq and intravital microscopy (IVM)(13,27,28), the mechanical expansion of the sagittal suture of a skeletally mature 2 month old mouse was demonstrated to be able to induce proliferation of the sagittal suture cSSCs. Finally, by means of microcomputed tomography (μCT), histological evaluations showed that the mechanical expansion sustains full regeneration of a e-CSD created in the parietal bone, and mouse genetics approaches showed that the regeneration process is mediated by Wnt signaling. Importantly, by further showing that Prx1/Prrx1 expressing cells are present in human calvarial sutures, the studies indicate that the shown mouse findings could potentially be translated to humans.

### Results

Single Cell RNA-Seq Profiling Identifies a Significantly High Number of cSSCs in the Calvarial Sutures of 4 Day Old Mice Versus Older Mice.

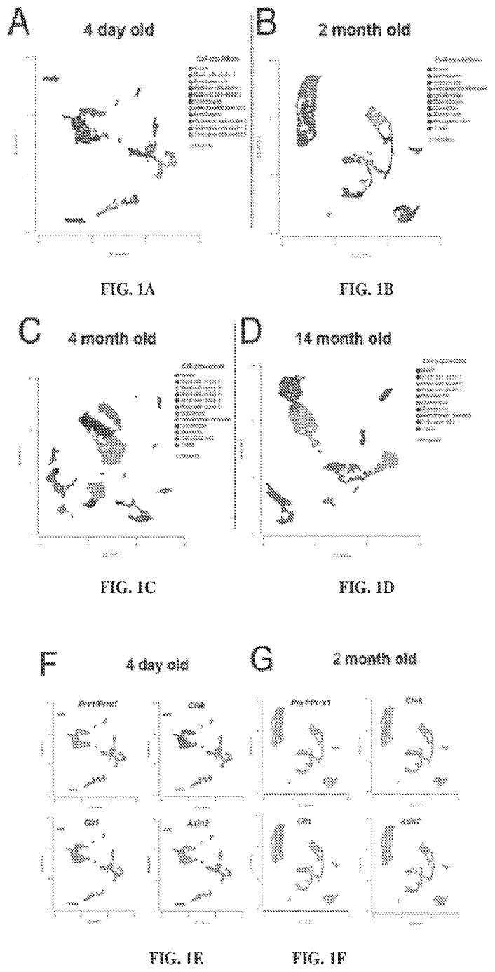

To evaluate and identify different populations of cells in the calvarial sutures of young and older mice, single cell RNA-sequencing analyses (scRNA-seq) was performed. Sutures explanted from 4 day old mice were utilized, an age when the suture is actively and naturally expanding, and sutures explanted from skeletally mature 2 month old, 4 month old, and 14 month old mice, representing ages when the sutures are functionally closed and have matured into synarthroses (29). In sutures of 4 day old mice, an unbiased cluster analysis of all the isolated cells identified various cell clusters, which include cell types of the hematopoietic lineage, epithelial lineage, and osteogenic lineage (FIG. 1A) (please, refer to Materials and Methods for the methodology utilized for the classification of the clusters). Three clusters, named osteogenic cells cluster 1, 2, and 3, make up all the cells of osteogenic lineage. The analysis of the calvarial sutures of 2 month old, 4 month old, and of 14 month old mice also reveals the presence of cells of the hematopoietic lineage and of the osteogenic lineage (FIGS. 1B-1D). However, at these ages, only one cluster containing a small number of cells could be identified as constituent of the osteogenic lineage. A quantification of the total osteogenic cells across all ages (indicated as percentage of the total cells evaluated, representing a more accurate quantitative parameter since the total number of cells loaded in each scRNA-seq assay variates from experiment to experiment) confirms that in the sutures of 4 day old mice the osteogenic cells are ˜44% of all the cells, whereas in 2 month old mice they decrease to ˜5% and in 4 months old and 14 month old they become less than 0.3% of all cells (Table 1). Conversely, non-osteogenic cells increase with aging, spanning from 56% in 4 day old mice to ˜95% in 2 month old mice, up to more than 99% in 4 month and 14 month old mice (Table 1).

TABLE 1
Quantification of osteogenic and non-osteogenic cell lineages (absolute numbers
counted in each sample and percentages of the total cells within each sample).

| Cells | 4 day old | 2 month old | | 4 month old | | 14 month old | |
|---|---|---|---|---|---|---|---|
| Non-osteogenic | 1288 (56.17%) | 3011 | (95.31%) | 6298 | (99.84%) | 5053 | (99.78%) |
| cells | | | | | | | |
| Osteogenic cells | 1005 (43.83%) | 148 | (4.69%) | 10 | (0.16%) | 11 | (0.22%) |

The scRNA-seq data to distinguish and quantify was interrogated, across the four different ages, cells expressing Prx1/Prrx1, Ctsk, Axin2, and Gli1, the four cSSCs markers independently identified by different groups of investigators (13-16), The cluster analysis indicates that these four cSSCs markers identify cells within the osteogenic cell clusters of all ages, and that these cells significantly diminish as the mice age (FIGS. 1E-1H). The quantitative analysis (Table 2) more specifically shows that in sutures of 4 day old mice a significant fraction of the total cells express Prx1/Prrx1 (˜41%) and Ctsk (˜48%), whereas cells expressing Gli1 and Axin 2 are less represented (˜10% and ˜8%, respectively). Differently, Prx1/Prrx1, Ctsk, Axin2, and Gli1 expressing cells are almost absent in the functionally closed sutures of all the older mice (values spanning from ˜1-2% in 2 month old mice to 0.2-1% in 14 month old mice), confirming that cSSCs significantly diminish in older mice. Collectively, these data indicates that expression of Prx1/Prrx1, Ctsk, G11, and Axin2 markers identify cells with similar gene expression profiles, suggesting that these cells are all representative of the cSSCs population. Furthermore, the data shows a correlation between the elevated number of cSSCs and the actively expanding sutures of 4 day old mice.

TABLE 2
Quantification of cells expressing Prx1/Prrx1, Ctsk, Gli1, and Axin2
(absolute numbers counted in each sample and percentages of the total
cells within each sample). 4 day old mice: total cells 2293 (n =
8); 2 month old mice: total cells 3159 (n = 5); 4 month old mice:
total cells 6308 (n = 5); 14 months old mice; total cells 5064 (n = 6).

| Cells | 4 day old | 2 month old | 4 month old | 14 month old |
|---|---|---|---|---|
| Expressing | 948 (41.34%) | 50 (1.58%) | 21 (0.33%) | 11 (0.22%) |
| Prx1/Prrxl | | | | |
| Expressing Ctsk | 1111 (48.45%) | 49 (1.55%) | 54 (0.85%) | 20 (0.39%) |
| Expressing Gli1 | 238 (10.37%) | 2 (0.06%) | 3 (0.05%) | 3 (0.06%) |
| Expressing Axin2 | 183 (7.98%) | 62 (1.96%) | 73 (1.15%) | 58 (1.14%) |

Overlapping Expression of Prx1/Prrx1, Ctsk, Gli1, and Axin2 is Observed in Proliferating cSSCs of the Calvarial Sutures.

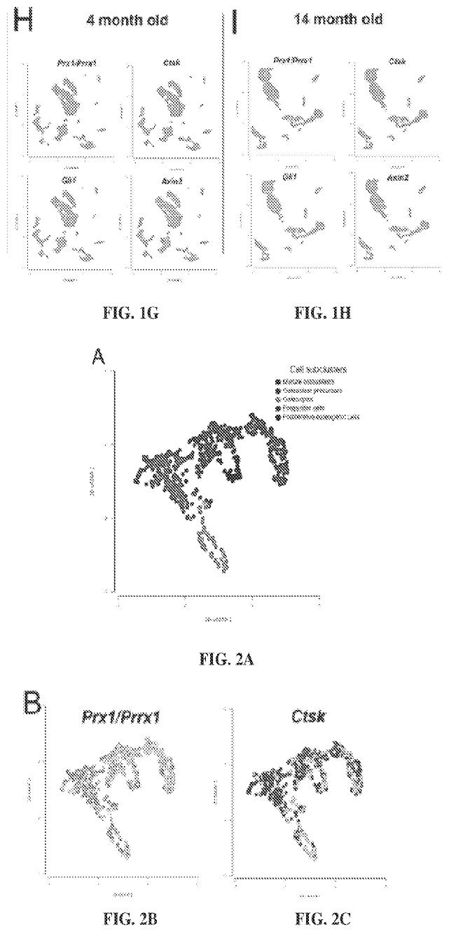

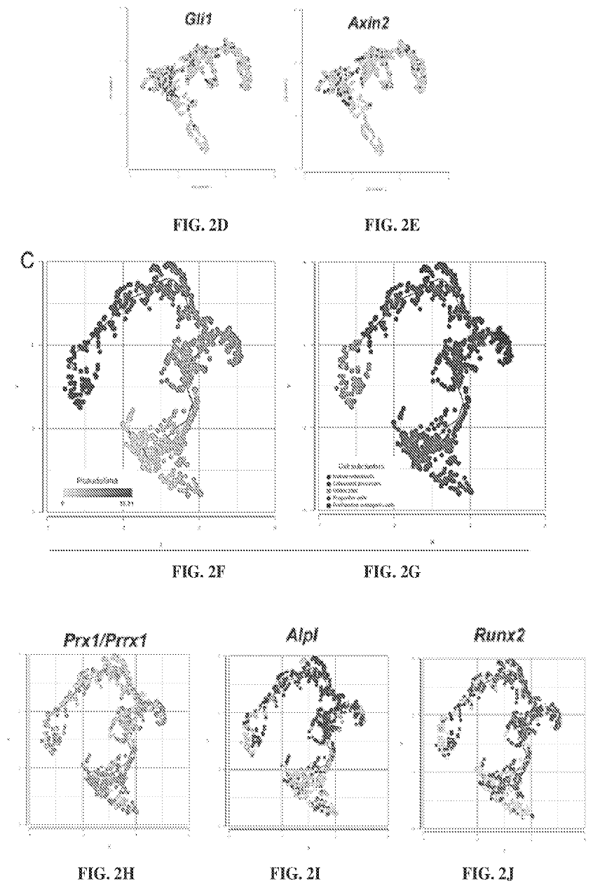

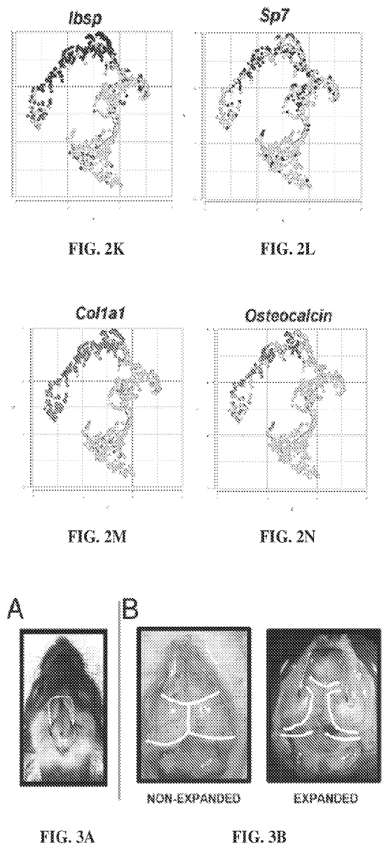

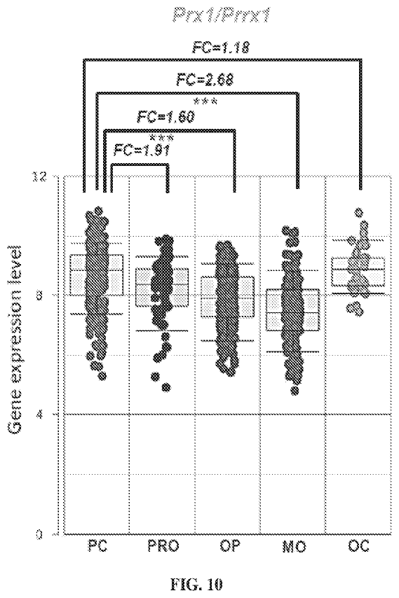

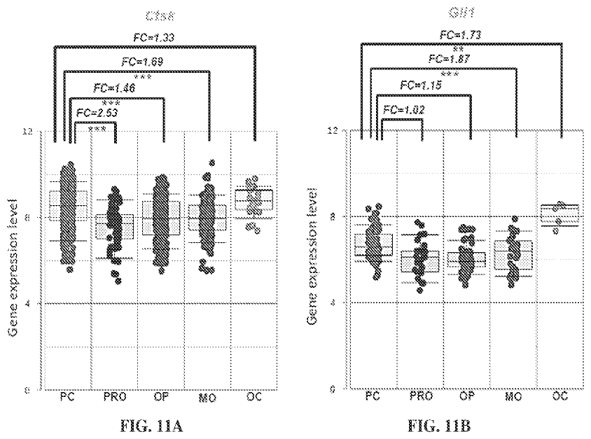

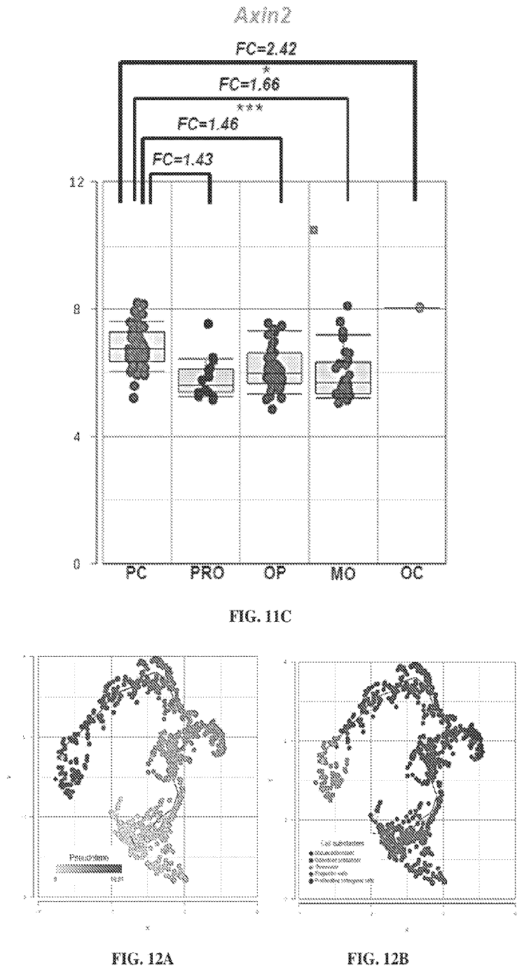

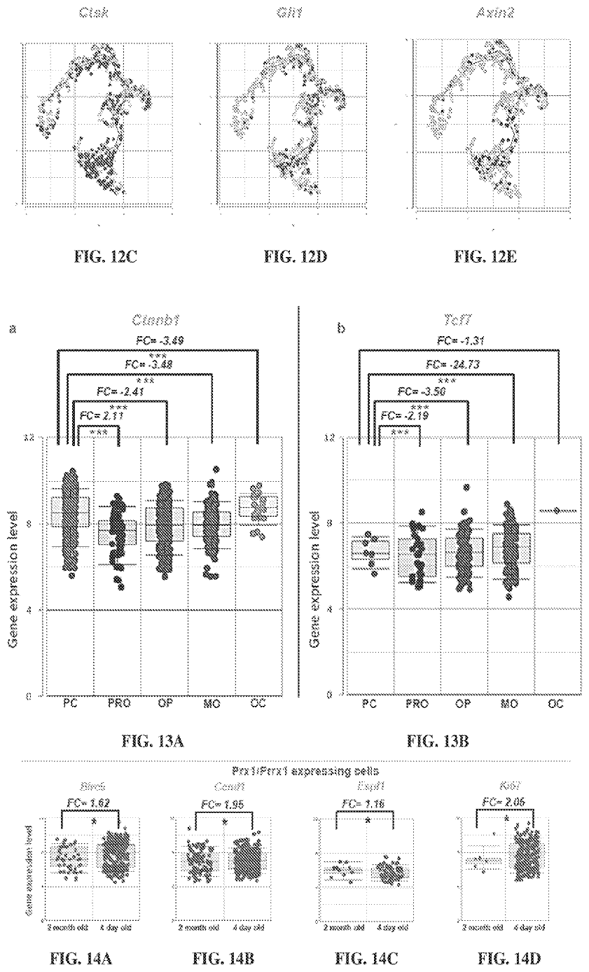

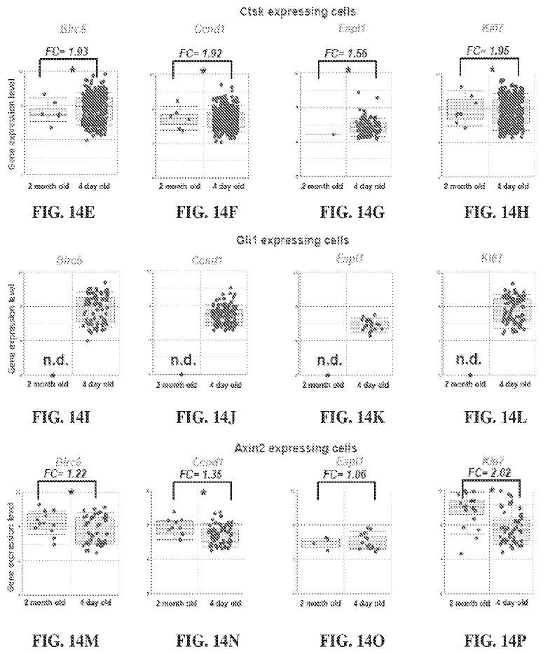

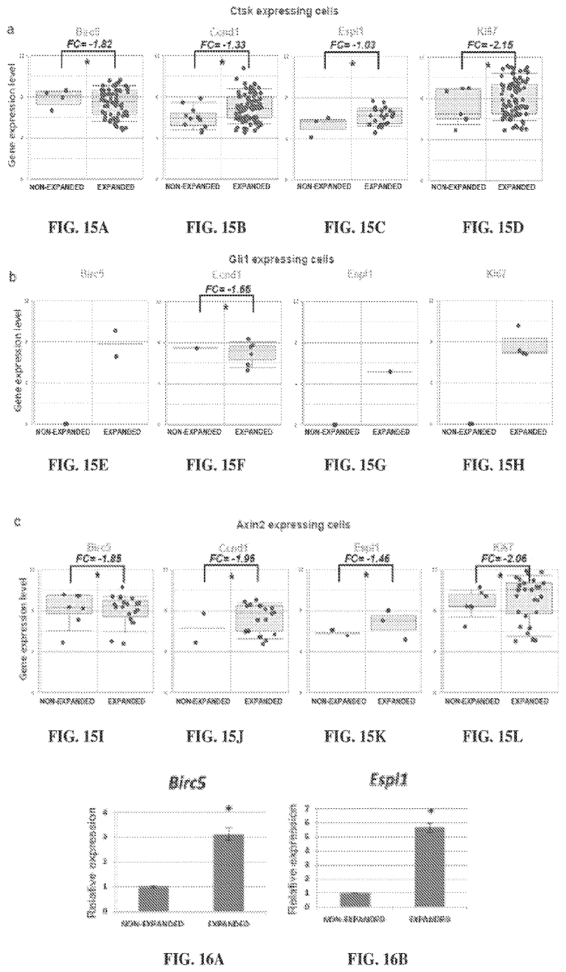

To confirm that expression of Prx1/Prrx1, Ctsk, Gli1 and Axin2 identify cells representative of the same cSSCs population, a re-clustering analysis of the osteogenic cells of the sutures of 4 day old mice was performed. This analysis identified 5 different subclusters of cells: 1) Progenitor cells (PC), 2) Proliferative osteogenic cells (PRO), 3) Osteoblast precursors (OP), 3) Mature osteoblasts (MO), and 4) Osteocytes (OC)(FIG. 2A)(please, refer to Materials and Methods for the methodology utilized for the classification of the subclusters). Expression of Prx1/Prrx1, Ctsk, Gli1 and Axin2, significantly overlaps with, and is mainly detectable within, the progenitor cells and, in part, in the osteoblast precursors (FIGS. 2B-2E). A dot plot quantification of the expression of Prx1/Prrx1 in each of the subclusters confirms that Prx1/Prrx1 is highly expressed in the progenitor cells subcluster (FIG. 10). Similar trends are observed in the dot plots quantification of the expression of Axin2, Ctsk, and Gli1 (FIGS. 11A-11C). To validate the sub clustering analysis and to assess the grade of differentiation of the various subclusters, a pseudotime analysis was performed and visualized the distribution of the subclusters along the obtained trajectory (FIGS. 2F-2G). This analysis identified the Progenitor cell subcluster as the earliest subcluster, while the Mature osteoblasts and the Osteocytes subclusters are projected at later time points, as the latest subclusters (FIGS. 2F-2G). When the expression of Prx1/Prrx1 with the pseudotime trajectory was overlapped, it was found that Prx1/Prrx1 to be mainly expressed within the early subclusters (FIGS. 2H-2N). Similar trends were observed for the expression of Ctsk, Gli1, and Axin2 (FIGS. 12D-12D). A quantitative evaluation of the Ctsk, Gli1, and Axin2 expressing cells within the early subclusters (Progenitor cells and the Proliferative osteogenic cells) shows that the vast majority of these cells (from 92% to 100%) co-express Prx1/Prrx1 (Table 5).Then, confirming that Pr1/Prrx1, Ctsk, Gli1, and Axin2 are most represented in early differentiation stages of the osteoblastic lineage, their expression to the expression of genes that associated to more differentiated cells of the osteoblastic lineage was compared. Data show that these genes (Alp1, Runx2, Ibsp, Sp7, Co11a1, and Osteocalcin) are mainly expressed further down into the trajectory (FIGS. 2H-2N). Since activation of Wnt signaling induces osteoblastic differentiation in cSSCs (13,28), the level of expression of D-catenin and Tcf7 across the subclusters (FIGS. 13A-13B) was further evaluated. This analysis showed a reduced expression of β-catenin and an almost undetectable expression of Tcf7 in the Progenitor cells subcluster, confirming that Wnt signaling is downregulated in undifferentiated cells and upregulated as cells move into their differentiation path. Collectively, these data indicate that the expression of Prx1/Prrx1, Ctsk, Gli1, and Axin2 identify a population of undifferentiated cells. Finally, to assess whether the undifferentiated cells of the sutures are proliferating, the scRNA-seq analysis to visualize the expression of Ki67, Esp11, Ccnd1, and Birc5, four genes commonly utilized to assess cell proliferation (30-33) was interrogated. This analysis confirms that expression of the proliferation genes is mainly detected within the early subclusters, in which Prx1/Prrx1, Axin2, Ctsk, and Gli1 expressing cells are highly represented (FIGS. 14A-14P). Collectively, these results indicate that expression of Prx1/Prrx1, Axin2, Ctsk, and Gli1 identifies undifferentiated and proliferating osteogenic cells within the expanding calvarial sutures of 4 day old mice.

TABLE 5
Quantitative evaluation of Ctsk expressing cells, Gli1 expressing
cells, and Axin2 expressing cells that co-express Prx1/Prrx1
within the Progenitor cells and the Proliferative osteogenic
cells subclusters of the sutures of 4 day old mice.

| | | Proliferative Osteogenic |
|---|---|---|
| Cells | Progenitor Cells | Cells |
| Expressing Prx1/Ctsk | 216/228 (94.73%) | 51/53 (96.22%) |
| Expressing Prx1/Gli1 | 74/77 (96.10%) | 27/28 (96.42%) |
| Expressing Prx1/Axin2 | 55/60 (91.66%) | 14/14 (100%) |

Expansion of the Sagittal Suture Induces Proliferation of the sSSCs

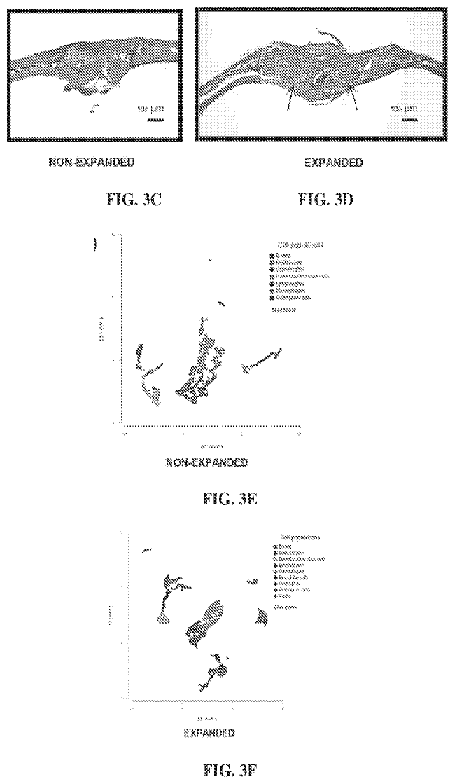

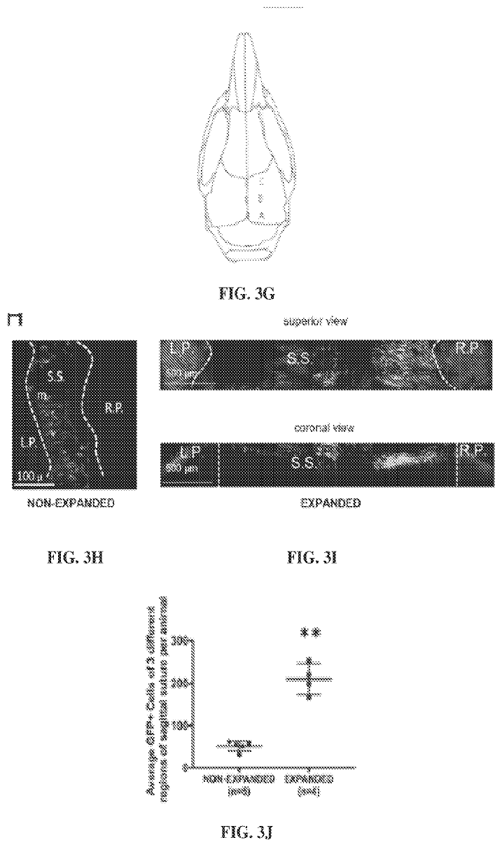

Since the naturally expanding sutures of the 4 day old mice are enriched in cSSCs, it was next hypothesized that a mechanical expansion of a functionally closed suture of skeletally mature 2 month old (8 week old) mice can reverse engineer what naturally occurs in 4 day old mice. More specifically, the mechanical expansion was hypothesized that can induce proliferation of the cSSCs. To test this hypothesis 2 month old mice were utilized, expanded their sagittal suture as previously described (34), and collected the tissue samples after 7 days of expansion (FIGS. 3A-3C). A histological examination confirmed that, after 7 days, the sagittal suture is effectively expanded (FIGS. 3D-3E). Then, scRNA-seq was utilized to analyze the cellular composition and the gene expression profile of cells of the non-expanded sutures (control, mock surgery) and the expanded sutures (test). As indicated by the UMAPs, the cell cluster analysis identifies a cluster of osteogenic cells in both the expanded and the non-expanded sutures (FIGS. 3F-3G). However, compared to the osteogenic cells of the non-expanded sutures, the osteogenic cells of the expanded sutures quadruplicate after 7 days of expansion (Table 3). On the contrary, after expansion, non-osteogenic cells diminish only by a small percentage (from approximately 99% to 95%) (Table 3). More specifically, the cells expressing Prx1/Prrx1 quadruplicate (from 1.4% to 5.2%), that cells expressing Ctsk triplicate (from 2.3% to 6.9%), and that cells expressing Axin2 or Gli1 duplicate (from 0.8% to 1.6% and from 0.1% to 0.2%, respectively) (Table 4). Then, to test whether the observed cell increase was specific for the cSSCs expressing Prx1/Prrx1, Ctsk, Axin 2, and Gli1, cells expressing Runx2, Sp7 (Osterix), and Osteocalcin (Bglap) were also quantified, which represent cells more differentiated along the osteoblastic lineage. Data indicate that, contrary to the sSSCs, the more differentiated cells decrease in expanded sutures, with cells expressing Osteocalcin decreasing up to almost ⅓ of the original number (Table 4).

Table 3. Quantification of osteogenic cells: scRNA_seq data is interrogated to quantify osteogenic and non-osteogenic cells of the expanded and non-expanded sutures (absolute numbers counted in each sample and percentages of the total cells within each sample).

TABLE 3
Quantification of osteogenic cells: scRNA_seq data is interrogated
to quantify osteogenic and non-osteogenic cells of the expanded
and non-expanded sutures (absolute numbers counted in each sample
and percentages of the total cells within each sample).

| Cells | Non-Expanded | Expanded |
|---|---|---|
| Non-osteogenic cells | 1480 (98.7%) | 3566 (95.4%) |
| Osteogenic cells | 18 (1.3%) | 169 (4.6%) |

TABLE 4
Quantification of cells expressing specific genes: scRNA_seq data
is interrogated to quantify cells expressing Prxl/Prrxl, Ctsk, Gli1,
Axin2, Runx2, Sp7 (Osterix), or Osteocalcin (Bglap) of the expanded
and non-expanded sutures (absolute numbers counted in each sample
and percentages of the total cells within each sample).

| | | |
|---|---|---|
| Cells | Non-expanded | Expanded |

| | | | | |
|---|---|---|---|---|
| Expressing Prxl/Prrxl | 22 | (1.47%) | 194 | (5.20%) |
| Expressing Ctsk | 35 | (2.33%) | 259 | (6.93%) |
| Expressing Gli1 | 2 | (0.13%) | 9 | (0.24%) |
| Expressing Axin2 | 13 | (0.86%) | 62 | (1.66%) |
| Expressing Runx2 | 278 | (18.55%) | 491 | (13.14%) |
| Expressing Sp7 | 14 | (0.93%) | 27 | (0.72%) |
| Expressing Osteocalcin | 492 | (32.84%) | 476 | (12.74%) |

An independent experiment, utilizing intravital microscopy (IVM) to quantify green fluorescent Prx1/Prrx1 expressing cells in non-expanded and expanded sagittal sutures of 2 month old Prx1-creER-EGFP mice (35), confirms the quantitative scRNA-seq analysis. In fact, when number of green fluorescent cells were quantified in three different locations along the non-expanded and expanded sagittal sutures of 2 month old mice, it was found that green fluorescent cells quadruplicate upon expansion (FIGS. 3H-3J).

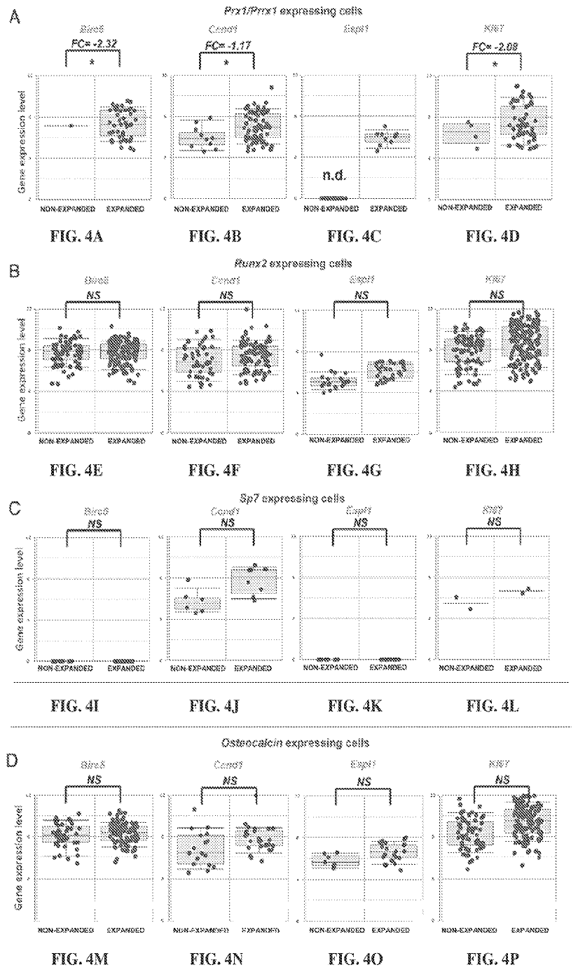

Finally, to test whether the increase of the number of cCCSs cells during suture expansion is due to proliferation activity, the scRNA-seq data was utilized and quantified the expression of Birc5, Ccnd1, Esp11, and Ki67 in cells of the expanded and of the non-expanded sutures (FIGS. 4A-4D). Data indicate that compared to Prx1/Prrx1 expressing cells of the non-expanded sutures, Prx1/Prrx1 expressing cells of the expanded sutures present with higher level of expression of all 4 genes (FIGS. 4A-4D). Similar results were observed in Ctsk, the Gli1,and the Axin2 expressing cells (FIGS. 15A-15L). On the contrary, more osteoblastic-differentiated cells expressing Runx2, Sp7, or Osteocalcin presented similar levels of expression of Birc5, Ccnd1, Esp11, and Ki67 in both non-expanded and expanded sutures (FIGS. 4E-4P).

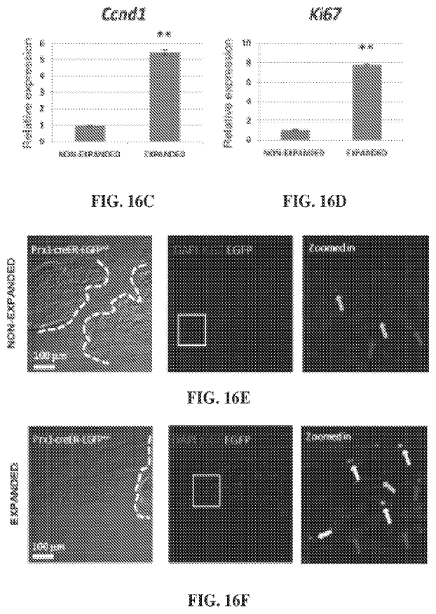

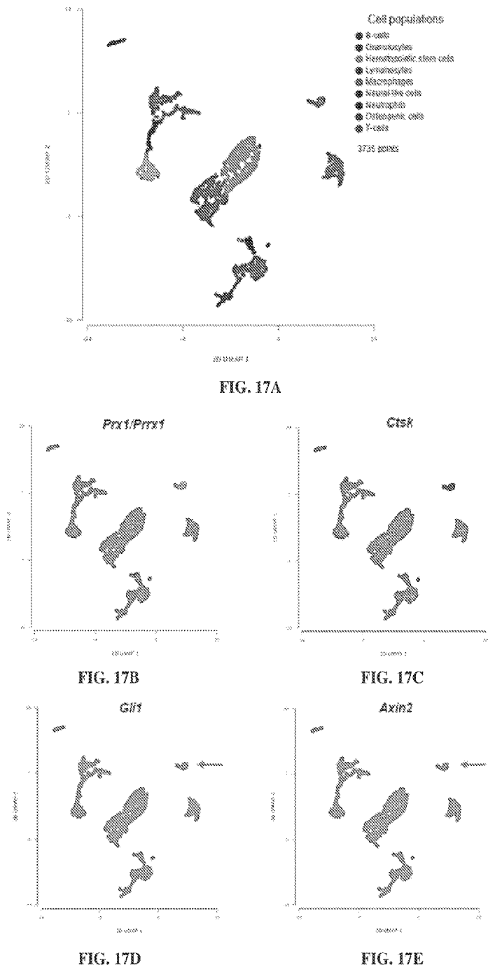

An independent evaluation, using quantitative PCR and in situ hybridization, performed two days after expansion also confirmed that Prx1/Prrx1 expressing cells proliferate during the mechanically induced expansion of the sutures (FIGS. 16A-16F).

Importantly, since Rindone et al (36) recently reported that the creation of a subcritical defect of 1 mm in diameter in the parietal bone can stimulate a significant expansion of the cSSCs, further the creation of the two 0.25 mm of diameter “anchoring holes” of the expansion devise was analyzed to determine whether it could, per se, induce any significant increase in the number of the cSSCs. To this end, the number of osteogenic and non-osteogenic cells of the 2 month old non surgery mice (as in Table 1) was compared with the number of osteogenic and non-osteogenic cells of the 2 month old mock surgery mice (no expansion devise inserted, as in Table 1), and observed no increment in the percentage of the osteogenic cells after the mock surgery. This is also the case for the Prx1/Prrx1, Ctsk, Gli1, and Axin2 expressing cells (compare Table 2 with Table 4).

Overall, these data indicate that mechanical expansion of the sagittal sutures increases the number and induces proliferation of the cSSCs.

cSSCs of the Mechanically Expanded Sutures and cSSCs Cells of the Naturally Expanding Sutures Present with Similar Gene Expression Profiles.

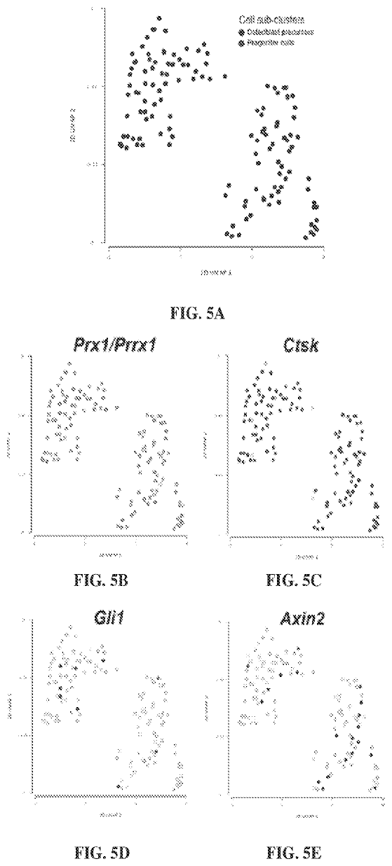

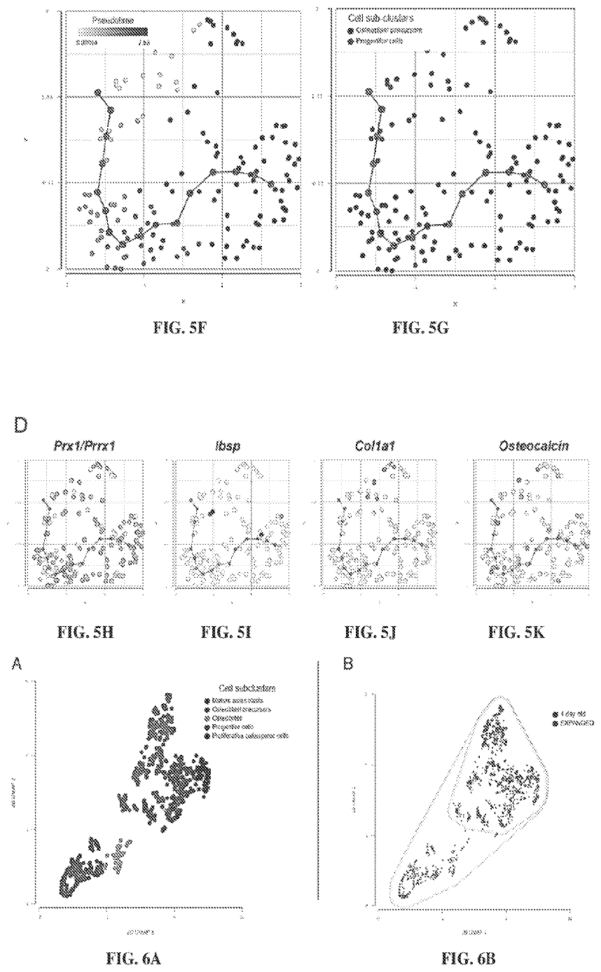

To assess the similarity between the cSSCs of the mechanically expanded sutures of the 2 month old mice and the cSSCs of the naturally expanding sutures of the 4 days old mice, the cluster analysis of the mechanically expanded sutures was first evaluated. This analysis shows that, similar to what was observed for the cells of the 4 day old sutures (see FIGS. 1A-1H), cells expressing Prx1/Prrx1, Ctsk, Gi1, and Axin2 of the mechanically expanded sutures are almost exclusively located in the osteogenic cells cluster (FIGS. 17A-17E). Then, a re-clustering analysis of the mechanically expanded osteogenic cells was performed. This analysis identified only 2 different subclusters of cells: the Progenitor cells subcluster and the Osteoblast precursors subcluster (FIG. 5A). Expression of Prx1/Prrx1, Ctsk, Gli1 and Axin2 is detectable in both subclusters (FIGS. 5B-5E). To assess the grade of differentiation of the two identified subclusters, a pseudotime analysis was performed and visualized the distribution of the subclusters along the obtained trajectory (FIGS. 5F-5G). Similar to what was found in the naturally expanding sutures of 4 day old mice, the Progenitor cells as the earliest subcluster were identified, while the Osteoblast precursors are projected at later time point (FIGS. 5F-5G). As expected, when the expression of Prx1/Prrx1 was overlapped with the pseudotime trajectory, Prx1/Prrx1 was found to be expressed in both subclusters (FIGS. 5H-5K), whereas Ibsp, Co11a1, and Osteocalcin, markers of more differentiated osteoblastic cells, are almost undetectable (FIGS. 5H-5K). Confirming that, in the mechanically expanding sutures, the expression of Prx1/Prrx1 overlaps with the expression of Ctsk, Gli1, and Axin2, cells, a quantitative evaluation of the cells expressing these genes in the Progenitor cells subcluster shows that, the vast majority of these cells (from 96% to 100%) co-express Prx1/Prrx1 (Table 6). A similar result was observed in the naturally expanding sutures (see Table 1). Finally, to confirm that the Progenitor cells of the mechanically expanding sutures are similar to the Progenitor cells of the naturally expanding sutures, the subclusters analysis was repeated combining the data from both samples (FIGS. 6A-6B). First, this analysis does not identify any additional subcluster, indicating that there is consistency of clustering between the two samples (FIG. 6A). Second, the data indicate that the Progenitor cells and the Osteoblast precursors of the mechanically expanding sutures overlap with the Progenitor cells and the Osteoblast precursors of the naturally expanding sutures (FIG. 6B) Collectively, these data indicate that cSSCs of the mechanically expanding sutures present with a gene expression profiles that resemble that of the sSSCs of the naturally expanding sutures of 4 days old animals.

Table 6. Quantitative evaluation of Ctsk expressing cells, Gli 1 expressing cells, and Axin 2 expressing cells that co express Prx 1/Prrx 1 within the Progenitor cells of the mechanically expanded sutures.

TABLE 6
Quantitative evaluation of Ctsk expressing cells, Gli 1 expressing
cells, and Axin 2 expressing cells that co express Prx 1/Prrx 1
within the Progenitor cells of the mechanically expanded sutures.

| Cells | PROGENITOR CELLS |
|---|---|
| Expressing Prx1/Ctsk | 57/59 (96.61%) |
| Expressing Prx1/Gli1 | 6/6 (100%) |
| Expressing Prx1/Axin2 | 6/6 (100%) |

### Suture Distraction Enhances Regeneration of Calvarial Critical Size Bone Defects

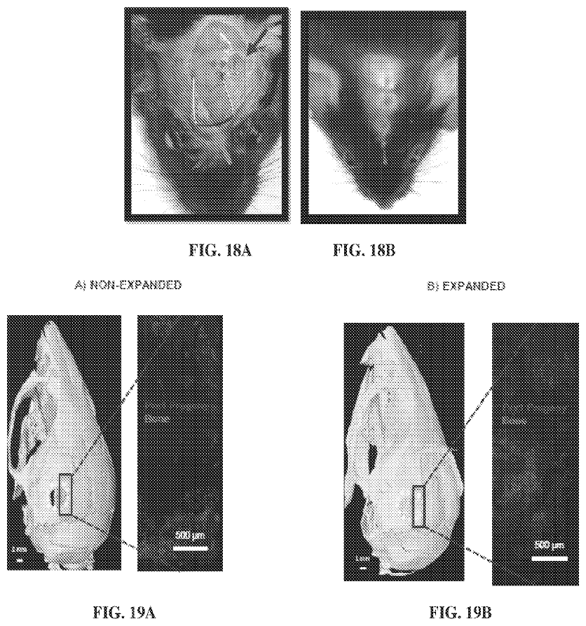

Since the calvaria of newborn mice is enriched of cSSCs, which resemble, at least in terms of gene expression, the cSSCs of the mechanically expanded sutures, and since newborn mice can fully regenerate calvarial bone defects, it was hypothesized that the mechanical expansion of the functionally closed sagittal suture of 2 month old (8 week old) mice could reverse engineer the spontaneous expanding suture of the newborn mice and could, consequently, sustain the complete regeneration of a e-CSD created in the parietal bone of these skeletally mature mice. To test this hypothesis, simultaneously to the expansion of the sagittal suture, a e-CSD within the parietal bone of the mouse calvaria was created, 3 mm lateral from the sagittal sutures and 1 mm mesial to the lambdoid suture (FIGS. 18A-18B). In control mice, the two small holes that would hold the expansion devise, as well as a e-CSD, were created in the same locations as in the test mice, but no expansion devise was inserted.

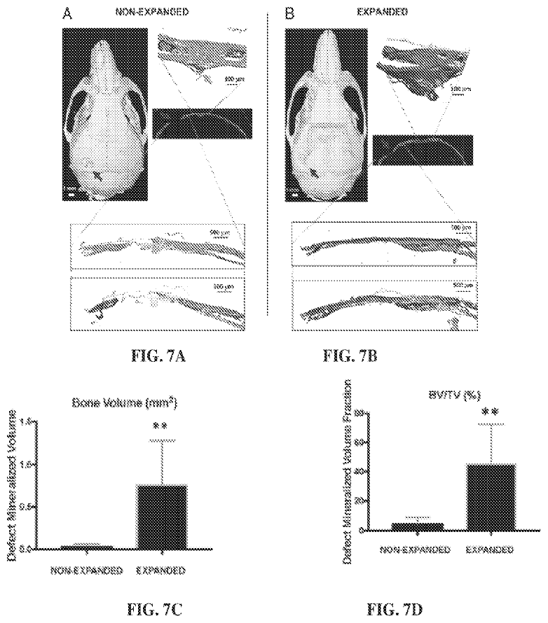

Sixty days after surgery, the e-CSDs of the control group showed limited amount of regenerated bone (FIG. 7A). On the contrary, the e-CSDs of the test group regenerated up to ˜100% of the missing bone (FIG. 7B). Micro-computed tomography (μCT) quantification confirmed that the bone volume (BV) and the bone fraction of bone volume over total volume (BV/TV) of the e-CSDs created simultaneously with the suture expansion are significantly higher than the BV and BV/TV of the e-CSDs created in control mice (FIGS. 7C and 7D). As expected, since it was previously shown that progeny of the cSSCs expressing Prx1/Prrx1 is responsible for regeneration of calvarila bone defects (13), a lineage tracing analysis performed in Prx1-creER-EGFP;tdTOMATO mice confirmed that the defect in the test mice is regenerated by these cells (FIGS. 19A-19B).

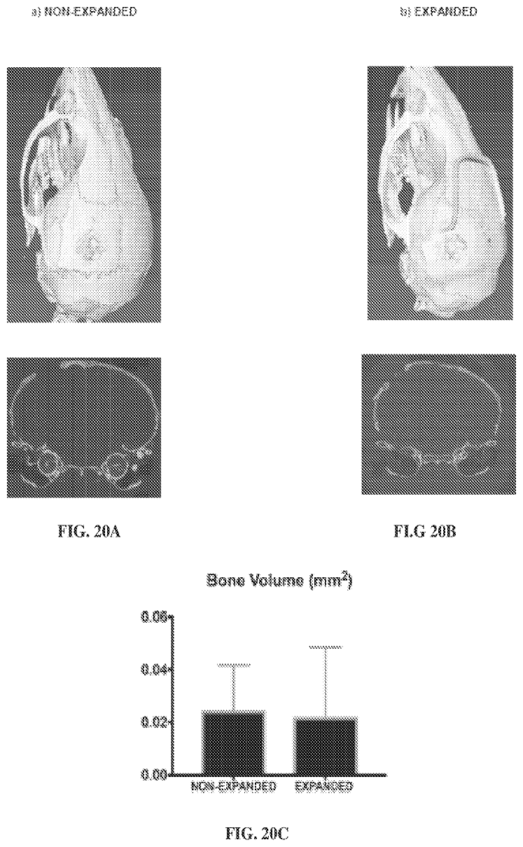

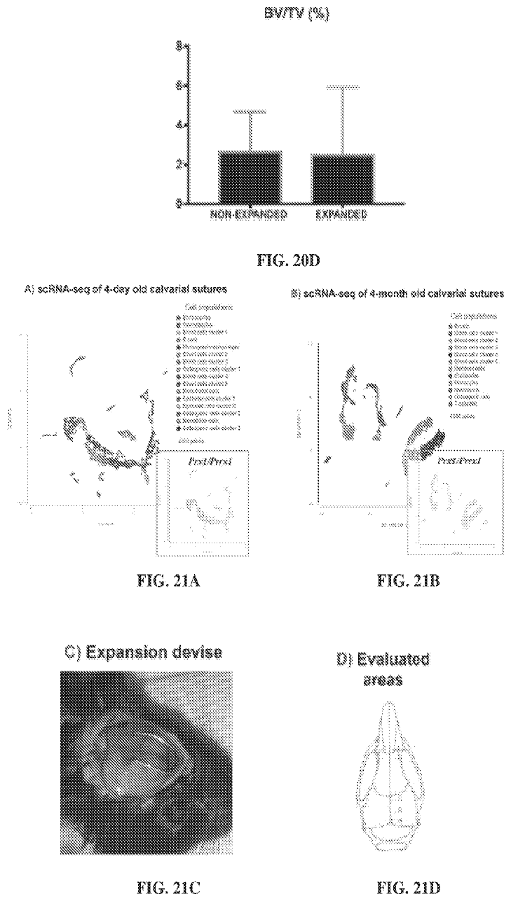

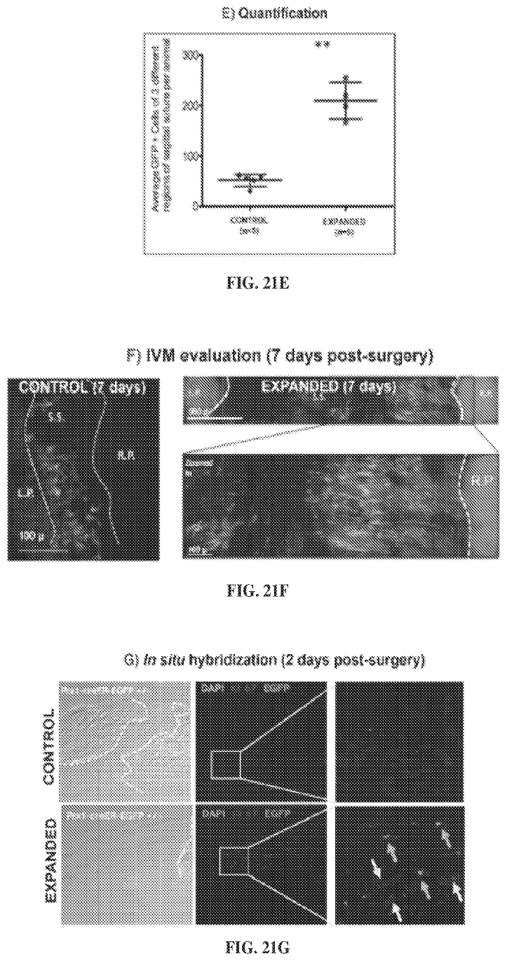

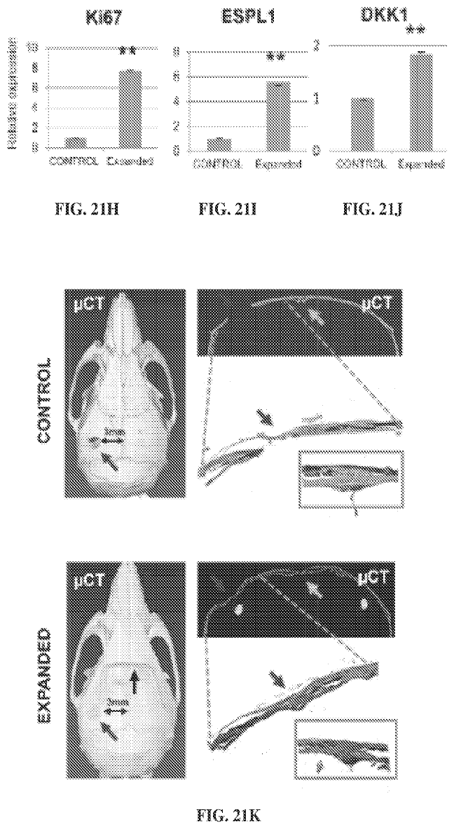

Expansion of a functionally closed suture, by means of induced proliferation of the pre-existing cSSCs, can sustain regeneration of calvarial bone defect otherwise unable to heal. Importantly, since regeneration does not occur in 10 month old mice (FIGS. 20A-20D), which represents an age in between the 4 month old and the 14 month old ones analyzed by scRNA-seq, the suture expansion-sustained regeneration was also propose that can occur only when a certain minimum number of preexisting cSSCs is present with the suture.

### Wnt Signaling Regulates Prx1/Prrx1 Expressing Cells of the Expanding Suture During The Regeneration of the Calvarial Critical Size Bone Defects

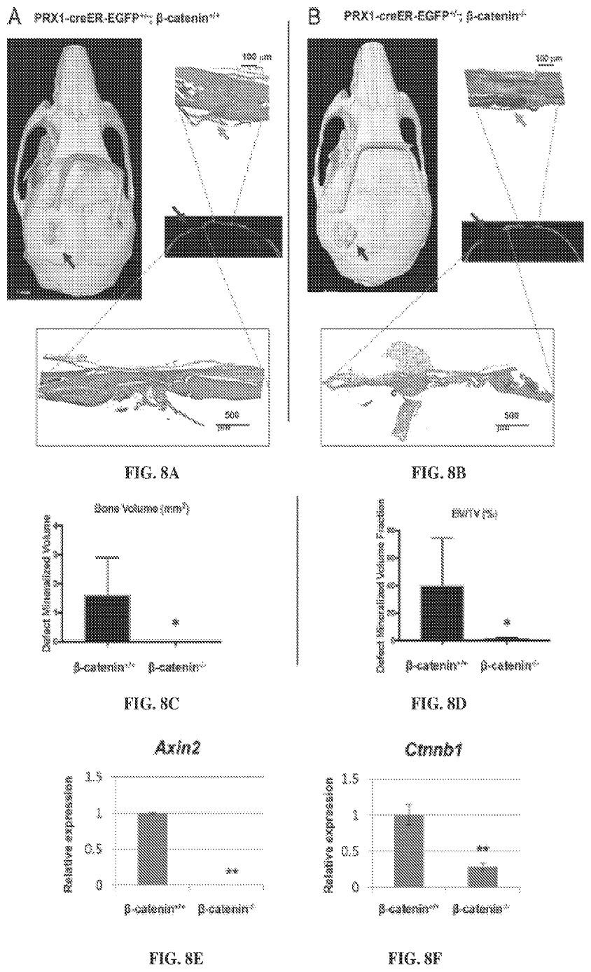

Since previous studies have shown that Prx1/Prrx1 expressing cells are Wnt-responsive cells (13,28), and since the scRNA-seq analysis of the expanding sutures of 4 day old mice shows that Wnt signaling is upregulated in differentiating cells of the osteoblastic lineage (FIGS. 13A-13B), next to investigate whether Wnt signaling in Prx1/Prrx1 expressing cells influences the suture expansion-sustained regeneration of the e-CSDs. To this end, 2 month old PRX1-creER-EGFP; β-catenin mice was utilized to conditionally inactivate j-catenin, and therefore canonical Wnt signaling, by means of cre recombinase (creER) in Prx1/Prrx1 expressing cells during expansion and during the regeneration process. As indicated by the μCT rendering and by the histological analysis of the defects (FIGS. 8A and 8B), it was found that the tamoxifen-induced genetic blockade of Wnt signaling in Prx1/Prrx1 expressing cells significantly impairs the capacity of the e-CSD to regenerate during expansion of the suture. The remodeling of the sagittal suture upon expansion was also impaired by the blockade (FIG. 8B). μCT quantification of the regenerated bone in the e-CSDs of 0-catenin inactivated mice revealed a reduction of the regenerated bone when compared to mice with active canonical Wnt signaling (control), with significant difference in BV and BV/TV (FIGS. 8C and 8D). The effective inactivation of Wnt signaling in Prx1/Prrx1 expressing cells was validated by analyzing the expression of Axin2 (a Wnt target gene) and β-catenin in FAC-sorted EGFP+ cells obtained from tamoxifen treated PRX1-creER-EGFP; β-catenin mice (FIGS. 8E-8F). On the basis of these data, canonical Wnt signaling is required during the suture expansion-sustained bone regeneration.

### Prx1/Prrx1 Expressing Cells are Located in the Expanding Human Calvarial Sutures.

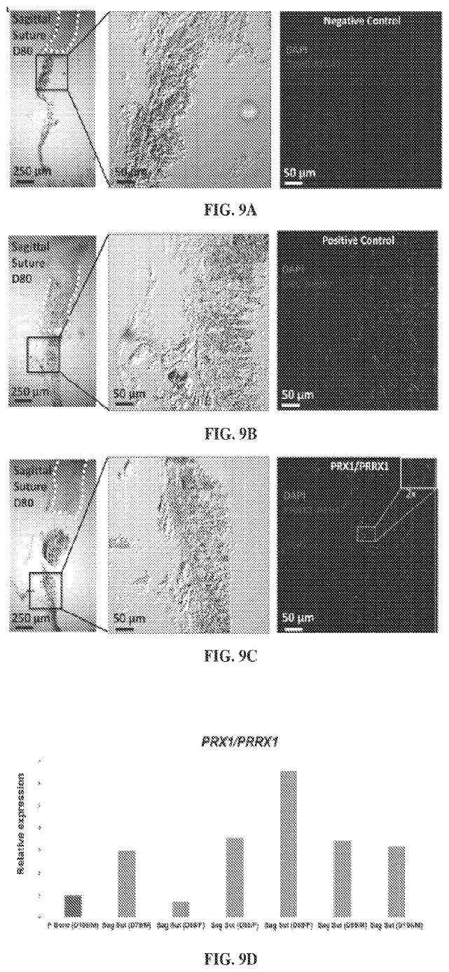

To test the translational significance of the findings observed in the mouse model, it was tested whether Prx1/Prrx1 is expressed in cells of the human sagittal suture. Previous studies in humans have shown that mutation of Prx1/Prrx1 or deletion of chromosome 1q23.3-q25.1 (the portion of the chromosome 1 that carries the human Prx1/Prrx1 gene) results in pre- and post-natal growth retardation, with microcephaly, micrognathia, and other skeletal malformations (37-39), thus suggesting that Prx1/Prrx1 may be expressed in the human calvarial sutures. Confirming that Prx1/Prrx1 is expressed in the human calvarial sutures, the in situ hybridization in sagittal suture of a human fetus 80 days post-conception shows expression of Prx1/Prrx1 in cells across the sutures (FIGS. 9A-9C), giving additional evidence of their role in calvarial development. To further confirm that Prx1/Prrx1 is highly expressed in cells of the human calvarial sutures, a quantitative PCR of the Prx1/Prrx1 gene in human primary cells obtained from the parietal bone of a human fetus (180 days post-conception) was performed and in human primary cells obtained from the fetal sagittal sutures of 6 different individuals (at various ages, from 79 days to 108 days post-conception). Results indicate that, compared to the parietal bone cells, sagittal suture cells express higher levels of Prx1/Prrx1 (in 5 out of the 6 tested samples) (FIG. 9D). Prx1/Prrx1 is expressed in cells of the human calvaria expanding sutures.

### DISCUSSION

Recent studies in the field of craniofacial bone biology have identified the calvarial sutures as reservoirs of skeletal stem cells expressing Prx1/Prrx1, or Ctsk, GIi1, and Axin2 (13-16). Specifically, the presence of postnatal skeletal stem cells expressing Prx1/Prrx1 within the calvarial sutures has been described and their requirement for calvarial bone regeneration (13), showing that the regeneration process is abrogated when a significant number of Prx1/Prrx1 expressing cells is ablated by means of a targeted expression of Diphtheria toxin. Transplanting sutures carrying traceable fluorescent Prx1/Prrx1 expressing cells, it was also shown that the calvarial bone regeneration occurs by means of their progeny. Building upon these studies, now it was shown for the first time that there is a significant overlap of expression of Prx1/Prrx1 in Ctsk, Gli1, and Axin2 expressing cells, indicating that expression of Prx1/Prrx1, Axin2, Ctsk, and Gli1 identifies the cSSCs population of the calvarial sutures.

While most of the current approaches to bone regenerative therapies focus on transplantation of bone competent cells, or on implantation of osteoconductive or osteoinductive biomaterials (40), here it was posited that such approaches, which are not exempt of health risks, may not be necessary if the regenerative potential of the native cSSCs is fully exploited. Since in children up to 2 years of age, with expanding osteogenically active calvarial sutures, regeneration of calvarial bone defects occurs naturally and without therapeutic aids, (17,18), it was hypothesized that the expanding sutures, with their high content of cSSCs, are responsible for this extraordinary regeneration potential. Therefore, using the skeletally mature 2 month old (8 week old) mouse calvaria as a model, it was aimed at demonstrating that if an otherwise functionally closed suture is artificially expanded, its content of cSSCs increases, and complete regeneration of a bone defect, even critical in size and remotely located from the suture, can occur. A scRNA-seq analysis of the cells of the calvarial sutures, performed in mice of different ages, from 4 day old up to 14 month old mice, demonstrated that the naturally expanding sutures of the 4 day old mice are highly enriched with cSSCs. Subsequently, it was demonstrated that a tensile force applied to a mature functionally closed suture can induce an enrichment in the number of the cSSCs and can sustain the regeneration of calvarial bone defects otherwise unable to regenerate. Therefore, the artificial expansion of a functionally closed, skeletally mature, suture can be utilized to harness the cSSCs and foster regeneration of calvarial bone defects. Importantly, the studies also show that the suture expansion strategy has limitations, since it is not effective in 10 month old mice, when the number of resident cSSCs is expected to be limited. This limitation is probably due to the cSSCs present in the 10 month old sutures, which may be either too limited in number or senescent to be able to proliferate up to a sufficiently higher number able to sustain the regeneration process. Thus, the suture expansion-sustained regeneration strategy has a limited temporal window of efficacy, although still effective in skeletally mature 2 month old mice.

Various mechanisms can explain the increase in the number of the cSSCs that were observe in the mechanically expanded suture. For instance it has been shown that progenitor cells directly sense the biophysical signals and transform them into a chemical response, a process known as mechanotransduction (41). In fact, mice lacking PKD1 or PKD2, two mechanotransduction modulators, develop abnormal suture patterning in response to mechanical stress (42,43). It is also possible that, during expansion, osteogenic cells respond to factors produced and secreted by neighboring cells or released from the extracellular matrix within their niche (44). For instance, similar to what happens to skeletal stem cells of the mandible upon distraction osteogenesis (45), a focal adhesion kinase (FAK) signaling pathway may regulate fate reprogramming, ultimately inducing proliferation of the PRX1+ cells. Studies showing that Prx1/Prrx1 expressing cells directly contribute to load-induced bone formation (46) further support this mechanotransduction theory. While identifying the molecular mechanisms of the mechanical induced proliferation of the cSSCs is beyond the scope of this work, based on the studies if an adequate number of these cells is present within the suture, a controlled tensile force applied to the suture can be utilized to induce their proliferation and sustain the regeneration of a remote calvarial bone defect.

Of interest in the present studies is the fact that the regeneration of a e-CSD occurs even when the e-CSD is positioned at a considerable distance from the suture. In fact, Park et al (26) have shown that the healing capacity of a e-CSD in the mouse calvaria decreases with increasing distance from the sutures, as e-CSD distant 1 mm from the sagittal suture are able to regenerate ˜50% of the missing bone while a e-CSDs distant 2 mm from the suture is able to regenerate only ˜20% of the missing bone (26). Thus, the suture expansion-sustained regeneration of a defect positioned 3 mm from the sagittal expanding sutures and 1 mm from the lambdoid suture is quite remarkable. The regeneration of remotely located defects also distinguishes the suture expansion-sustained regeneration from the traditional distraction osteogenesis, whereby bone formation is limited within the distraction site. While similar molecular mechanisms may regulate the osteogenic activity within the tension sites, the ability of the cSSCs, or their progeny, to reach a distant defect is regulated by cellular migrating mechanisms not necessarily activated in the distraction osteogenic site. Many hypotheses can be formulated to explain how the proliferating PRX1+ cells contribute to the regeneration of a remote calvarial bone defect. For instance, regeneration may occur by means of circulating or by means of directly migrating cSSCs that from the sutures reach the remote defect. Previous in vivo studies have demonstrated that tensile forces induce upregulation of genes associated with vascularization (34) and therefore one could speculate that the recruitment of osteoprogenitor cells in remote defects may occur through circulation, secondary to an enhanced neovascularization. Alternatively, it might be speculated that the application of a mechanical force in vivo may elicit a dynamic change in the structure of the extracellular matrix, or changes in the activity of soluble growth factors and cytokines, or may disrupt the direct cell-cell contacts to facilitate cell migration (47,48), facilitating the migration of the cSSCs, or their progeny, to the remote defect. Regardless of the mechanisms responsible for the cell migration and the remote regeneration process, the current study demonstrates that they depend on the activation of Wnt signaling within the Prx1/Prrx1 expressing cell during the regeneration process. This conclusion is validated by existing studies showing that Wnt signaling is involved with craniofacial development (49), as well as calvarial suture homeostasis (49,50)(28) and calvarial bone regeneration (51), and that is activated by tension forces applied to teeth during orthodontic treatments (52) and required for distraction osteogenesis of long bones (53).

With the goal of investigating the translational potential of the mouse studies, it was investigated whether expression of Prx1/Prrx1 could also be detected in cells of the human calvarial sutures. Similar to cells of the mouse sutures, cells of the human fetal sutures (in situ hybridization) and primary cells derived from the fetal sutures (in vitro qPCR assays) express Prx1/Prrx1. This result, along with the documented involvement of mutations of the Prx1 gene with craniofacial malformations (37,38)(54), proves that Prx1/Prrx1 expressing cells have a significant role in the craniofacial development and might, consequently, have a role in the regeneration of the human calvarial bone defects as well. Considering that the biology of the mouse calvarial sutures closely resembles the one described in humans (20-22), one may speculate that the suture expansion-sustained calvarial regeneration process that was described in mice should be also observed in humans. Clinical studies would be needed to verify this possibility. Yet, it was suggest that the translational meaning of the present studies is quite significant since, at least in mice, the suture expansion-sustained bone regeneration process does not require transplantation of osteogenic tissue or implantation of any biomaterial or scaffold within the bone defects. Since bone distraction devises are commonly utilized in humans to correct craniosynostosis and other craniofacial malformation (55,56), and since resorbable devises have been recently developed to eliminate the need for a second operative procedure for hardware removal (57), clinical studies could be performed to test this methodology. Such studies should also evaluate the limitations associated to the distance between the activated expanding suture and the healing site, as regeneration may significantly decrease beyond a certain distance threshold. Importantly, given the age-associated limitations that were observed in mice, future clinical studies should assess these limitations in humans as well. While no conclusion can be inferred because the maturational rate of mice does not linearly correlate with that of humans (58), one may estimate that a 2 month old age in mice corresponds to the age of adulthood in humans (58), thus suggesting that the suture expanding strategies could be useful in children over 2 years of age and in adults as well.

Finally, expanding on the calvarial clinical application, since Prx1/Prrx1 expressing cells are present within the periosteum of long bones and significantly contribute to the healing of long bone fractures (59,60), one may translate the results of the present calvarial studies to the regeneration of defects in long bones. For instance, special minimally invasive devises could be engineered to deliver a tensile tenting force to the periosteum of the long bones to induce activation of the periosteum and sustain the regeneration of otherwise non-healing fractures, even when they are remotely located (i.e. expansion of the diaphyseal periosteum for healing of the femur's head fractures).

In conclusion, sutures may be regarded as targetable autotherapy entities whose local stimulation can sustain regeneration of otherwise non-healing calvarial bone defects. Thus, the studies may lead to the development of more effective bone regenerating autotherapies for humans, whereby the endogenous healing capacity of each patient is fully exploited by harnessing the SSCs.

### Methods

### Animals

Experiments were conducted in compliance with the Guide for the Care and Use of Laboratory Animals at the University of Pittsburgh School of Dental Medicine (IACUC Protocol #: 20066890) and at the Harvard School of Dental School (IACUC Protocol #IS00000535). To optimize the quantification of the fluorochrome expression and minimize signal noise observed in female mice (13), only Prx1-creER-EGFP male mice were utilized for the IVM quantification studies. Accordingly, only male mice (C57BL/6) were utilized for the scRNA-seq studies. Male and female mice (C57BL/6 and Prx1-creER-EGFP;β-catenin) were randomly distributed in each group for the bone regeneration studies.

Single cell RNA sequencing Cells were isolated using collagenase digestions and scRNA-seq was performed using Chromium Next GEM Single Cell 3′ GEM, Library & Gel Bead Kit v3.1 (10X Genomics, USA) following the manufacturer's guidelines. The subsequent data analysis, including statistical evaluations, was performed using Partek® Flow® software, v10.0 (Partek, Inc., USA). Clusters and subcluster were identified first by using an unbiased bioinformatic approach, in which cell identities using the Partek® Flow® software's “Biomarker” function were attributed, obtaining a list of the top 25 most expressed genes by each cluster or subcluster (Supplemental Tables 3-10). Second, the expression of the cell identifiers obtained by Partek® with pre-existing and published data (61,62) were compared.

### Cell Isolation

Mice were euthanized following the IACUC guidelines and sagittal, coronal, and lambdoid sutures were manually dissected using a dissecting microscope. Upon dissection, and consistently for all samples, no more than ¼ mm of bone tissue surrounding the sutures was collected. Samples were incubated in 4 mL of collagenase solution (3.2 mg Collagenase Type 2, Worthington-Biochem, catalog #LS004176, 1 mL of Trypsin-EDTA, GIBCO, catalog #25200-056 and 3 mL of DPBS, GIBCO catalog #14190-144) for 20 minutes at 37° C., for 4 consecutive times. Cell suspensions derived from each incubation were pulled and centrifuged, and the obtained cell pellet was resuspended in 10 mL of media.

### Single-Cell RNA Sequencing

Freshly isolated cells obtained from the sutures (see cell isolation) of eight 4 day old, five 2 month old, five 2 month old NON-EXPANDED, six 2 month old EXPANDED, five 4 month old, and six 14 month old C57BL/6 wild type mice were utilized to perform the scRNA-seq using Chromium Next GEM Single Cell 3′GEM, Library & Gel Bead Kit v3.1 (Document number CG000204, 10X Genomics), following the manufacturer's guidelines. cDNA library QC and quantification were performed using Agilent 4150 TapeStation with High Sensitivity D1000 ScreenTape® Page. Sequencing was completed with Illumina Novaseq 6000, achieving ˜46000 to ˜66000 mean reads per cell.

### Data Analysis

Raw data in FASTQ format were processed using 10X Genomics Cell Ranger 3.1.0 to generate filtered feature-barcode matrices. The subsequent data analysis was performed using Partek® Flow® software, v10.0. Single cell counts were filtered to remove potential cell duplets and dead cell by excluding cells with <200 and >6000 genes and cells with a mitochondrial gene percentage of >10%. Normalized counts on a per-cell basis were expressed in count per million (CPM). Graph-based analysis and successive evaluation with uniform manifold approximation and projection (UMAP) were executed over the first 11 principal components, with resolution parameters of 0.5 for all.

### Cell Cluster and Subcluster Identity Recognition

The identity of cells belonging to each detected cluster or subcluster was uncovered by means of two approaches. First an unbiased bioinformatic approach was employed, in which cell identities were attributed using the Partek® Flow® software, v10.0 “Biomarker” function, obtaining a list of the top 25 most expressed genes by each cluster or subcluster (Tables 7-14). Second, the expression of the cell identifiers obtained by Partek® Flow® software, v10.0 with pre-existing and published data (23,62) were compared.

### Statistical Analysis

Statistical analysis of scRNA-seq data was performed using the Hurdle model embedded in the Partek® Flow® software, v10.0 package (Partek Incorporated, USA).

### Trajectory Analysis

Trajectory analysis and pseudotime calculation were performed using the Monocle2 package of the Partek® Flow® software, v10.0 package (Partek Incorporated, USA), using default parameters.

TABLE 7
List of the genes utilized to identify the cell clusters of the calvarial sutures of 4
day old mice. The 25 most significantly expressed genes are reported for each cluster.

| Osteogenic cells | Epithelial cells | Epithelial cells | Blood cells cluster | |
|---|---|---|---|---|
| cluster 2 | cluster 2 | cluster 1 | 1 | B-cells |
| Prrx2 | 2610528A11Rik | Stmn3 | Hbb-bs | Vpreb3 |
| Cavin3 | Cyp2f2 | Ttc9b | Hba-a1 | Mzb1 |
| Fstl1 | Ifi202b | Bex2 | Hba-a2 | Cd79a |
| Mfap2 | Ehf | Act16b | Gm42418 | Cd79b |
| Fermt2 | Fxyd3 | Scg5 | Hbb-bt | Ighm |
| Aebp1 | Pof1b | Chga | Pabpc1 | Ptprcap |
| Itm2a | Serpinb5 | Pou3f4 | Psap | Rhoh |
| Clec11a | S100a14 | Gng3 | Cst3 | Pou2af1 |
| Tpm2 | Dapl1 | Gm17750 | Ndufs2 | Cd19 |
| Mest | Foxq1 | Rundc3a | Atp6v1f | Fcrla |
| Col6a1 | Aqp3 | TagIn3 | Vamp8 | Il7r |
| Igsf10 | Col17a1 | Crmp1 | Cltc | Igkc |
| Gpx7 | Eppk1 | Scg3 | St13 | Slamf6 |
| Htra1 | Trim29 | Gdap1 | Ndufb6 | Cecr2 |
| Twist1 | Fgfbp1 | Igsf21 | Neat1 | Gimap1 |
| Prrx1 | Barx2 | Rtn1 | Csnk2b | Chchd10 |
| Gpc3 | Dsp | Sp8 | Wdr26 | Tnfrsf13c |
| Grb10 | Ckmt1 | Stmn2 | Napa | Pax5 |
| Olfml3 | Rab25 | Nol4 | Psmd2 | Siglecg |
| Aspn | Mpzl2 | Soga3 | Nsd3 | Tifa |
| Cdkn1c | Anxa8 | Nsg2 | Dbi | Cd37 |
| Itgb5 | Tfap2b | Tubb4a | Ptpn1 | Arl5c |
| Ptn | Urah | Ly6h | Zcchc6 | Slamf7 |
| Col6a2 | Pkp1 | Igfbpl1 | Ctsb | Klhl6 |
| Gpx8 | Fermt1 | Tox3 | Cfdp1 | Spib |

List of the genes utilized to identify the cell clusters of the calvarial sutures of 4
day old mice. The 25 most significantly expressed genes are reported for each cluster.

| Osteogenic cells | Osteogenic cells | | Hematopoietic | | |
|---|---|---|---|---|---|
| cluster 1 | cluster 3 | Granulocytes | stem cells | Endothelial cells | Lymphocytes |
| Col22a1 | Malat1 | Gm5416 | Fcnb | Gria2 | Ifi207 |
| Slc36a2 | Gm42418 | Ly6g | Ms4a3 | Nrxn3 | Clec4a3 |
| Car3 | Col1a1 | Slpi | 1700020L24Rik | Dlx6os1 | Ms4a6d |
| Cgref1 | Col1a2 | Mmp8 | Prss57 | Dcx | Trem2 |
| Col11a2 | AY036118 | Fpr2 | Cldn15 | Map2 | Ctss |
| Smpd3 | Meg3 | Slfn1 | Gca | Celf4 | Csf1r |
| Col24a1 | Kcnq1ot1 | 2010005H15Rik | Ffar2 | Elavl3 | Cd68 |
| Slc13a5 | mt-Atp6 | Cxcr2 | Cebpe | Adarb2 | Ms4a6c |
| Satb2 | mt-Co3 | Mcemp1 | Clec5a | Nrxn1 | C3ar1 |
| Pcsk6 | Egr1 | Retnlg | Gm1604a | Gm3764 | Cd83 |
| Mamdc2 | Col3a1 | Fpr1 | Trem3 | Meis2 | Cd300c2 |
| Sema3b | Peg3 | Stfa2 | Serpinb1a | 5330434G04Rik | Adgre1 |
| Col13a1 | Col16a1 | Ceacam10 | Mgst2 | Cadps | Lrrc25 |
| Sgms2 | Timp3 | Asprv1 | Rab44 | Grin2b | Msr1 |
| Ifitm5 | mt-Cytb | Ifitm6 | Hsd11b1 | Il1rapl1 | Aif1 |
| Cdh2 | mt-Nd4 | Chil1 | Gatm | Etv1 | Tnfrsf11a |
| Fabp3 | Plagl1 | Pilra | Adgrg3 | Gad2 | Clec4a1 |
| Itga10 | mt-Co1 | Plbd1 | Abca13 | Miat | Gpr183 |
| Ptgis | Eln | Wfdc21 | Rgs18 | Xist | Fcgr1 |
| Slc8a3 | Col5a1 | Cd33 | Slco4c1 | C130071C03Rik | Ly86 |
| Cpe | mt-Co2 | AA467197 | Clec12a | Zbtb20 | Clec4n |
| Kcnk1 | Ptprd | Mrgpra2b | Nkg7 | Lrrc7 | Irf5 |
| Cd200 | Igfbp5 | Trem3 | Tuba4a | Hbb-bs | Cyth4 |
| Cadm1 | Lars2 | Lrg1 | Ly6c2 | Hba-a1 | Cd86 |
| 2310022B05Rik | Fbxl7 | Cd177 | Lbp | Dlgap1 | Ccl9 |

TABLE 8
List of the genes utilized to identify the cell clusters of the calvarial sutures of 2
month old mice. The 25 most significantly expressed genes are reported for each cluster.

| Granulocytes | Lymphocytes | B-cells | Myeloid cells | Macrophages |
|---|---|---|---|---|
| Cd177 | S100a4 | Igkc | Fcrls | Cxcl2 |
| Ifitm6 | Ccr2 | Cd79b | P2ry12 | Il1b |
| Mmp9 | Ms4a4c | Cd79a | C3ar1 | Ccl6 |
| Syne1 | Fn1 | Vpreb3 | Gpr34 | Clec4d |
| Ly6g | Ccl9 | Ebf1 | Slc2a5 | Acod1 |
| Wfdc21 | Ms4a6c | Ly6d | C1qc | Slc7a11 |
| Mmp8 | Vcan | Ighm | C1ga | Nlrp3 |
| Retnlg | Clec4a3 | Ms4a1 | Mertk | Csf3r |
| S100a9 | Ifi207 | Pax5 | Hpgds | Cd300ld |
| Adpgk | F13a1 | Pou2af1 | C1gb | Pla2g7 |
| Anxa1 | Clec4a1 | Bach2 | Slco2b1 | Il1r2 |
| Plbd1 | Pld4 | BE692007 | Ltc4s | Entpd1 |
| Ngp | Mcub | Fcrla | Adap2 | Dusp1 |
| Pglyrp1 | Ctss | Iglc2 | Sall1 | Trem1 |
| S100a8 | Ms4a6b | Iglc3 | Crybb1 | Plek |
| Lcn2 | Ahnak | Cecr2 | Tmem119 | Ccr1 |
| Mmp25 | Msr1 | Spib | Pdgfb | S100a6 |
| Itgam | Itgb7 | Cd72 | Cx3cr1 | Hdc |
| Pygl | Ifi30 | Cd19 | Hpgd | Retnlg |
| Hdc | Pid1 | Cd74 | Itgb5 | Hcar2 |
| Mcemp1 | Lrp1 | Rag1 | Tanc2 | Cebpb |
| Chil1 | Rassf4 | Ikzf3 | Rnase4 | Slc15a3 |
| Fpr2 | Ctsc | Chst3 | Cttnbp2nl | H2-Q10 |
| Itgb2l | Mafb | Fcmr | Olfml3 | Il1rn |
| Ltf | Crip1 | Tnfrsf13c | Frmd4b | Cxcr2 |

List of the genes utilized to identify the cell clusters of the calvarial sutures of 2
month old mice. The 25 most significantly expressed genes are reported for each cluster.

| Hematopoietic stem | | | | |
|---|---|---|---|---|
| cells | Monocytes | Erythrocytes | T-cells | Osteogenic cells |
| Ms4a3 | Ass1 | Alas2 | Cd7 | Apod |
| Fcnb | Ms4a6c | Hbb-bs | Ccl5 | Ptn |
| Cebpe | F13a1 | Snca | Il2rb | Cald1 |
| 1700020L24Rik | Al506816 | Gypa | Trbc2 | Igfbp7 |
| Rrm2 | Ccr2 | Slc4a1 | Klrd1 | S100a16 |
| Prss57 | Lgals1 | Tspo2 | Ctsw | Col4a1 |
| Cdca8 | Idh2 | Rhd | Itk | Nfib |
| Cdc25a | S100a10 | Trim10 | Cd3g | Serpinh1 |
| Pclaf | Kcnn4 | Rec114 | Ms4a4b | Wwtr1 |
| Cdk1 | Mcm3 | Ank1 | Gimap4 | Neo1 |
| Mogat2 | Gria3 | Spta1 | Cd3e | Htra1 |
| Smc2 | Prdx4 | Slfn14 | Trbc1 | Fermt2 |
| Spc24 | Cd48 | Cldn13 | Skap1 | Timp3 |
| Agpat2 | S100a4 | Pdzk1ip1 | Trac | Zfhx4 |
| Gca | Rassf4 | Ermap | Gimap3 | Epn2 |
| Tacc3 | Cks1b | Rhag | Txk | Tjp1 |
| Spc25 | Mcm5 | Add2 | Sla2 | Nckap1 |
| Birc5 | Uck2 | Tmcc2 | Sh2d2a | Aebp1 |
| Top2a | Sdf2l1 | Sptb | Xcl1 | Ptprd |
| Kif11 | Nhp2 | Hemgn | P2ry10 | Plat |
| Lbp | Pola1 | Hba-a2 | Cox6a2 | Lhfp |
| Tmed3 | Itga1 | Fech | Ikzf2 | Ramp2 |
| Tpx2 | Rexo2 | Sox6 | Ccr9 | Mir100hg |
| Serpinb1a | Selenoh | Kel | H2-Q7 | Epas1 |
| Orm1 | Ybx3 | Paqr9 | Klre1 | Lamb2 |

TABLE 9
List of the genes utilized to identify the cell clusters of the calvarial sutures of 4
month old mice. The 25 most significantly expressed genes are reported for each cluster.

| Blood cells | | | Blood cells | Blood cells | Osteogenic |
|---|---|---|---|---|---|
| cluster 2 | Erythrocytes | T-cells | cluster 4 | cluster 5 | cells |
| Ltf | Hbb-bs | Ccl5 | Il1rn | Cxcr2 | Ighe |
| Ngp | Alas2 | Trbc2 | Acod1 | Mmp9 | Siglech |
| Abca13 | Snca | Cd3e | Clec4d | Fos | Jchain |
| AA467197 | Lars2 | Xcl1 | Entpd1 | Ptgs2 | Bglap |
| Syne1 | Hba-a1 | Il2rb | Fth1 | Osm | Ccr9 |
| Krt83 | Gypa | Gata3 | Slc7a11 | Trim30b | Bglap2 |
| Itgb2l | Hba-a2 | Trbc1 | Cd300ld | Il1b | Ighg2c |
| Ly6g | Hbb-bt | Trac | Il1r2 | Jaml | Klk1 |
| Cd177 | Slc4a1 | Skap1 | Slc15a3 | Csf3r | lghg2b |
| Acpp | Epb42 | Icos | Ccr1 | Cd300ld | Sh3bgr |
| Camp | Bpgm | Eomes | Tgm2 | Slfn1 | Den |
| Ifitm6 | Tspo2 | Sytl3 | Arg2 | Trem1 | Cox6a2 |
| Adpgk | Clk1 | Klre1 | Antxr2 | Btg2 | Igha |
| Ankrd22 | Pabpc1 | Itk | Cxcl2 | Plk3 | Gm21762 |
| St3gal5 | Spta1 | Ms4a4b | Ccl6 | Mmp8 | Sparc |
| Ldhc | AY036118 | Txk | Trem1 | Retnlg | Mgp |
| Plscr1 | Prxl2a | Cd3g | Hdc | Egr1 | Procr |
| Plbd1 | Arhgap45 | Thy1 | Basp1 | Nfam1 | Prss34 |
| Cdadc1 | Rhd | Serpinb9 | Srgn | Junb | Cpa3 |
| Lcn2 | BC005537 | Ctsw | Slc16a3 | Hacd4 | Serpinh1 |
| Chil1 | Rsrp1 | KIrc1 | Adam8 | Dusp1 | Mcpt8 |
| Anxa3 | H2-K1 | KIrc2 | Tpd52 | Vsir | Igkc |
| Anxa1 | Hbq1b | Gm2682 | Csf3r | Tmem154 | Myct1 |
| 1700047M11Rik | Slfn14 | Gimap3 | Clec4e | Ppp1r3b | Cd300c |
| Fpr2 | Cldn13 | Fasl | Plek | S100a6 | Igkv5-43 |

List of the genes utilized to identify the cell clusters of the calvarial sutures of 4
month old mice. The 25 most significantly expressed genes are reported for each cluster.

| Blood cells cluster | Hematopoietic | | | | |
|---|---|---|---|---|---|
| 3 | stem cells | B-cells | Neutrophils | Lymphocytes | Blood cells cluster 1 |
| S100a4 | Fcnb | Rag1 | Ctsg | Fcer2a | Rsph9 |
| Fn1 | Orm1 | Atp1b1 | Prtn3 | H2-DMb2 | Hist1h1a |
| Vcan | Mogat2 | Cpm | Mpo | Fcmr | Myl4 |
| Ms4a6c | Cebpe | Xrcc6 | Ms4a3 | Ighd | Uhrf1 |
| F13a1 | Spc25 | Fam129c | Prss57 | Ms4a1 | 1810059H22Rik |
| Msr1 | Inhba | Cecr2 | Cst7 | Scd1 | Gm37065 |
| Ccr2 | Cdc25a | Vpreb3 | FkbpI1 | Bank1 | Arntl |
| Ifi207 | Cdkn3 | Akap12 | Nkg7 | Cd74 | Cplx2 |
| Mcub | Hist1h3c | Dnajc7 | Rab38 | H2-Ob | 1700027J07Rik |
| Mafb | Nuf2 | Bcl7a | Srm | H2-Aa | Chchd10 |
| Ms4a6d | 1700020L24Rik | Sox4 | Nme4 | H2-Ab1 | Ddah2 |
| Aif1 | Rrm2 | Ebf1 | Sdf2l1 | H2-Eb1 | Igll1 |
| Itga1 | Hmmr | Cd79b | Cdk6 | Ly6d | Slamf7 |
| Cd300lg | Ulbp1 | Bach2 | Tspan4 | Ccr7 | Vpreb1 |
| Trem2 | Smc2 | Tifa | Abhd14a | Iglc3 | Vpreb3 |
| Clec4a3 | Cdk1 | Arl5c | Igfbp4 | H2-Oa | Zfpm1 |
| Pid1 | Mki67 | Fam53b | Tma16 | Cxcr5 | Mzb1 |
| Ms4a4c | Ube2c | Slamf6 | Nbdy | Mef2c | Il7r |
| Shtn1 | Asf1b | Rhoh | Vkorc1 | Iglc2 | Cecr2 |
| Ptpro | Arhgap19 | Fcrla | G6pc3 | Cd79a | Akap12 |
| Stxbp6 | Cdca8 | Chst3 | Gsto1 | Ly6a | Bcl7a |
| Arhgef10I | Kif15 | Cd72 | 1190007l07Rik | Cd83 | Pafah1b3 |
| Clec4a1 | Prc1 | Pafah1b3 | C1gbp | Gm31243 | Pou2af1 |
| Tnip3 | Tacc3 | Spib | Wdr12 | Ccr9 | Cenpm |
| Ctsc | Nusap1 | Blnk | Gar1 | H2-Eb2 | Pax5 |

TABLE 10
List of the genes utilized to identify the cell clusters of the calvarial sutures of 14
month old mice. The 25 most significantly expressed genes are reported for each cluster.

| | | | Hematopoietic stem | |
|---|---|---|---|---|
| Blood cells cluster 2 | Erythrocytes | Granulocytes | cells | B-cells |
| Ltf | Alas2 | Cxcl2 | Fcnb | Rag1 |
| Ifitm6 | Snca | Cd300ld | Ms4a3 | Atp1b1 |
| Krt83 | Apol11b | Clec4d | Prss57 | Fam129c |
| 1700047M11Rik | Hbb-bs | Acod1 | Mogat2 | Cecr2 |
| AA467197 | Rec114 | Il1b | Gatm | Akap12 |
| Ly6g | Gypa | Csf3 | 1700020L24Rik | Vpreb3 |
| Mmp25 | Hbq1b | Entpd1 | Cebpe | Bcl7a |
| Itgb2l | Trim10 | Ccl6 | Orm1 | Cd79b |
| Cd177 | Tspo2 | Slc7a11 | Cdc25a | Dnajc7 |
| Ngp | Spta1 | Il1r2 | Tmed3 | Xrcc6 |
| Lcn2 | Slc4a1 | Slc15a3 | Vcam1 | Sox4 |
| Syne1 | Dmtn | Ccr1 | Spc25 | Cd72 |
| Anxa1 | Epb42 | Trem1 | Rrm2 | Ebf1 |
| Adpgk | Sox6 | Cxcr2 | Cdk1 | Chst3 |
| Wfdc21 | Apol11a | Pla2g7 | Cdca8 | Spib |
| St3gal5 | Rhag | Hdc | Ncam1 | Tifa |
| Fpr2 | Rhd | Nlrp3 | Lbp | Bach2 |
| Mmp9 | Slfn14 | Dusp1 | Spc24 | Fam53b |
| Plbd1 | Sowaha | S100a6 | Cdca7 | Ly6d |
| Camp | Kel | Slc16a3 | Mgst2 | Ar150 |
| Chil1 | Fhdc1 | Mmp9 | Gca | Pafah1b3 |
| S100a8 | Ank1 | Plek | Smc2 | Chchd10 |
| S100a9 | Sptb | Retnlg | Birc5 | Fcrla |
| Itgam | Cldn13 | Ankrd33b | Igfbp4 | Mtss1 |
| Cybb | Tspan33 | Fth1 | Pclaf | Pou2af1 |

List of the genes utilized to identify the cell clusters of the calvarial sutures of 14
month old mice. The 25 most significantly expressed genes are reported for each cluster.

| | | | | Osteogenic |
|---|---|---|---|---|
| T-cells | Blood cells cluster 3 | Dendritic cells | Blood cells cluster 1 | cells |
| Ccl5 | Mafb | Fcer2a | Hist1h1a | Cd34 |
| Trbc2 | Vcan | H2-DMb2 | Rsph9 | Siglech |
| Cd3e | Clec4a3 | Ighd | Uhrf1 | Bst2 |
| Cd3g | Ifi207 | Gm31243 | Myl4 | P2ry14 |
| Bcl11b | Fn1 | Bank1 | 1700027J07Rik | Ptprs |
| Skap1 | S100a4 | Fcmr | 1810059H22Rik | Tsc22d1 |
| Il2rb | Ccr2 | Scd1 | Cenpm | Cox6a2 |
| Trbc1 | Mcub | H2-Aa | Nrgn | Ccnd1 |
| Itk | Lrp1 | H2-Ob | Igll1 | Angpt1 |
| Cd7 | Clec4a1 | H2-Eb1 | Cplx2 | Phgdh |
| Trac | Pid1 | Cd74 | Lockd | Muc13 |
| Cd28 | Cx3cr1 | H2-Ab1 | Slc16a1 | Ccr9 |
| Thy1 | Ms4a6c | Ms4a1 | Arntl | Rab38 |
| Sytl3 | F13a1 | Ccr7 | Gm37065 | Slc22a3 |
| Icos | Ctss | Cxcr5 | Vpreb1 | Myct1 |
| Pmepa1 | Ms4a4c | Cd83 | Sapcd2 | Tmem176b |
| Ms4a4b | Csf1r | Cd79a | Lef1 | Cd300c |
| Gimap3 | Ccl9 | H2-Eb2 | Pclaf | Srm |
| Klrd1 | Ahnak | Zfp318 | Chchd10 | Pmepa1 |
| Tcf7 | Pld4 | lgkc | Fbxo5 | Sdsl |
| Lck | Dusp3 | Ebf1 | Esco2 | Ybx3 |
| Txk | Msr1 | Mef2c | Vpreb3 | Hoxa7 |
| Ikzf2 | Plxnb2 | Gimap6 | Nek2 | Tmem176a |
| Ctla2a | Ifi30 | Cd22 | Ncapd2 | Flt3 |
| Sh2d2a | Rassf4 | Ralgps2 | Cecr2 | Rexo2 |

TABLE 11
List of the genes utilized to identify the cell clusters of the calvarial sutures of NON- EXPANDED (mock
surgery) 2 month old mice. The 25 most significantly expressed genes are reported for each cluster.

| | | Hematopoietic | | | | Osteogenic |
|---|---|---|---|---|---|---|
| Macrophages | Granulocytes | stem cells | B-cells | Erythrocytes | Lymphocytes | cells |
| Clec4d | Itgb2l | Ms4a3 | Cd2 | Hbb-bs | Ms4a6c | Ecrg4 |
| Ccl6 | Ltf | Fcnb | Cd79a | Hba-a1 | Pld4 | Ccn1 |
| Il1b | AA467197 | 1700020L24Rik | Ebf1 | Hba-a2 | Ahnak | Ccn3 |
| Il1r2 | Syne1 | Mogat2 | Gimap1 | Spta1 | Lgals1 | Igf2 |
| Acod1 | Ly6g | Gatm | Ptprcap | Nedd8 | Fn1 | Apod |
| Slc7a11 | Cd177 | Prss57 | Gimap3 | Pf4 | S100a4 | Ccn2 |
| Cd300ld | Adpgk | Cdca8 | BE692007 | Slc4a1 | Rassf4 | Slco1a4 |
| Csf3r | Mmp25 | Cdc25a | Ablim1 | Lnpep | Ctss | Slco1c1 |
| Ccr1 | 1700047M11Rik | Tacc3 | Gimap4 | Rsbn1l | Ifi30 | Cxcl12 |
| Ankrd33b | Ngp | Smc2 | Gimap5 | Gm42418 | Ly86 | Mgp |
| Slc15a3 | Camp | Cdk1 | Gimap6 | Ndufb7 | Bst2 | Ly6c1 |
| Trem1 | Plbd1 | Cdkn3 | Cd79b | Rbx1 | Scpep1 | Spock2 |
| Cxcl2 | Ceacam1 | Birc5 | Ccr7 | Inpp5d | Ccr2 | Npr3 |
| Cxcr2 | Acpp | Igfbp4 | Vpreb3 | Smap2 | Klf4 | Slc4a10 |
| Fth1 | Ifitm6 | Spc24 | Igkc | Usp34 | Mcub | Col8a1 |
| Entpd1 | Fpr2 | Top2a | Sh2d2a | Uqcr11 | H2-DMa | Gpx3 |
| Slc16a3 | Golim4 | Tpx2 | Skap1 | Srrm1 | Ifi207 | Ccn4 |
| Srgn | Cybb | Prc1 | Rag1 | Brd4 | Rgs10 | Serping1 |
| Retnlg | Chil1 | Cdca3 | Txk | Herc1 | F13a1 | Mir100hg |
| S100a6 | Abca13 | Ube2c | Cd72 | Ywhab | Pid1 | Ccn5 |
| Dusp1 | Ckap4 | Cebpe | Trbc2 | Cdkn1b | S100a10 | Rian |
| Tpd52 | St3gal5 | Cit | H2-Q6 | Tacc1 | Crip1 | Adgrf5 |
| Hdc | Rflnb | Cks1b | Pax5 | Cox6c | Slc25a4 | Itgbl1 |
| Grina | Cpne3 | Pclaf | Fcrla | Prrc2a | Ctsc | Ptx3 |
| Adam8 | Lcn2 | Cenpe | Il2rb | Tnrc6c | Nme2 | Plat |

TABLE 12
List of the genes utilized to identify the cell clusters of the mechanically EXPANDED calvarial
sutures of 2 month old mice. The 25 most significantly expressed genes are reported foreach cluster.

| | | Hematopoietic stem | | |
|---|---|---|---|---|
| Macrophages | Granulocytes | cells | B-cells | Neutrophils |
| Acod1 | Ltf | Fcnb | Rag1 | Ctsg |
| Clec4d | Ly6g | Cebpe | Cd79b | Dmkn |
| Il1b | Cd177 | Orm1 | Ebf1 | Cst7 |
| Slc7a11 | AA467197 | Gca | Cecr2 | Prtn3 |
| Cxcl2 | Ifitm6 | 1700020L24Rik | lgkc | Elane |
| Trem1 | Syne1 | Hist1h3c | Ly6d | Sdf2l1 |
| Hdc | Itgb2l | Nusap1 | Vpreb3 | Syce2 |
| Plek | Adpgk | Cdkn3 | Cd79a | Al506816 |
| Cd300ld | Ngp | Prc1 | Ighm | Kcnk12 |
| Il1r2 | Camp | Pclaf | Bcl7a | Gstm1 |
| Ccl6 | Mmp25 | Cdca8 | Chchd10 | Mpo |
| Csf3r | Plbd1 | Spc25 | Atp1b1 | Ms4a3 |
| Ccr1 | Fpr2 | Smc2 | Akap12 | Mcm3 |
| Retnlg | Chil1 | Kif11 | Spib | Plppr3 |
| Slpi | Wfdc21 | Kif15 | Cd72 | Gria3 |
| Cebpb | Krt83 | Cdk1 | Fam129c | Mcm5 |
| Slc16a3 | Acpp | Mki67 | Bach2 | Ass1 |
| Lilr4b | Pglyrp1 | Top2a | Pax5 | Gmnn |
| Srgn | Anxa1 | Mgst2 | Rhoh | Cks1b |
| Cxcr2 | Abca13 | Ly75 | Fcrla | Rfc5 |
| Mmp9 | Lcn2 | Birc5 | Dnajc7 | C1gbp |
| Entpd1 | 1700047M11Rik | Abca13 | Pafah1b3 | Selenoh |
| Grina | St3gal5 | Mogat2 | Tifa | Dctpp1 |
| Slc15a3 | Mmp9 | Ica1 | Sox4 | Exosc8 |
| Dusp1 | Cybb | Ube2c | Pou2af1 | Pa2g4 |

List of the genes utilized to identify the cell clusters of the mechanically EXPANDED calvarial
sutures of 2 month old mice. The 25 most significantly expressed genes are reported for each cluster.

| Lymphocytes | Osteogenic cells | T-cells | Neural-like cells |
|---|---|---|---|
| Ifi207 | Sfrp2 | Cd7 | Kif1a |
| Trem2 | Aspn | Trbc2 | Pex5l |
| Clec4a3 | Ecrg4 | Cd3g | Elavl3 |
| Msr1 | Postn | Il2rb | Gnao1 |
| Ms4a6d | Col6a3 | Gimap4 | Slc24a2 |
| Clec4a1 | Mgp | Cd3e | Stmn4 |
| Mafb | Col3a1 | Ccl5 | Mapk10 |
| Ctss | Pcsk5 | Ctla2a | Gfy |
| S100a4 | Col12a1 | Sh2d2a | Plekhb1 |
| Pid1 | Ogn | Ctsw | Snap25 |
| Ccl9 | H19 | P2ry10 | Stmn3 |
| Ms4a6b | Col6a1 | Klrd1 | Fstl5 |
| Ms4a6c | Lrrc17 | Gimap6 | Ncam2 |
| Csf1r | Cdh11 | Trbc1 | Myt1l |
| F13at | Fmod | Gimap3 | Soga3 |
| Ctsc | Prrx2 | Xcl1 | Chga |
| Pld4 | Gas1 | Skap1 | Ttll7 |
| Ifi30 | Fstl1 | Sla2 | Cntn4 |
| Lgmn | Col14a1 | Trac | Rims2 |
| Ccr2 | Itgbl1 | Lck | Aplp1 |
| Fcgr1 | Thbs2 | Ms4a4b | Limch1 |
| Ahnak | Npr3 | Ccr9 | Dtna |
| Aif1 | Mfap4 | Siglech | Tshz2 |
| Cxcl16 | Col6a2 | Klre1 | Scn9a |
| Cd68 | Fbn1 | Klrk1 | Bex2 |

TABLE 13
List of the genes utilized to identify the cell subclusters within the
osteogenic cells cluster of the calvarial sutures of 4 day old mice. The
25 most significantly expressed genes are reported for each cluster.

| | Osteoblasts | | | Proliferative |
|---|---|---|---|---|
| Mature osteoblasts | precursors | Osteocytes | Progenitor cells | osteogenic cells |
| Col22a1 | Nrgn | Gm42418 | Mfap4 | Pbk |
| Col11a2 | Tnc | Hbb-bs | Fbn1 | Depdc1a |
| Car3 | Pdzrn4 | mt-Co3 | Igf1 | Hmmr |
| Slc36a2 | Tpm2 | Hba-a1 | Rarres2 | Tpx2 |
| Smpd3 | Acta2 | mt-Co1 | Plpp3 | Mxd3 |
| Ibsp | Podnl1 | Hba-a2 | Gm13305 | Ckap2l |
| Cgref1 | Alpl | mt-Co2 | C1qtnf2 | Ccna2 |
| Sgms2 | S1pr1 | Malat1 | Fbln2 | Spc25 |
| Slc13a5 | Srpx2 | mt-Cytb | Fbln5 | Aurkb |
| Ifitm5 | Pak1 | Col1a1 | Fbn2 | Nusap1 |
| Cpe | Fgfr3 | mt-Atp6 | Col4a1 | Nuf2 |
| Col1a1 | Cnn3 | AY036118 | Serping1 | Anln |
| Col24a1 | Tagln2 | mt-Nd1 | Ndn | Bub1 |
| Cadm1 | S100a4 | mt-Nd4 | Meg3 | Ndc80 |
| Mamdc2 | Fat3 | Hbb-bt | Pmp22 | Kif11 |
| Cdh2 | Pdlim4 | Gm47815 | Cpxm2 | Shcbp1 |
| Sema3b | Fabp5 | Tpi1 | Ogn | Esco2 |
| Satb2 | Postn | Gm26720 | Igfbp4 | Pimreg |
| Pcsk6 | Tbcb | Gm45159 | Nid1 | Cdca8 |
| Col1a2 | Sncaip | Wwtr1 | Igfbp2 | Hist1h2ab |
| Ramp1 | Aprt | Txk | Ramp2 | Cenpe |
| Fabp3 | S100a13 | Tram1 | Sfrp2 | Kif15 |
| Kcnk1 | S100a10 | Ovgp1 | Col4a2 | Sgo2a |
| Prex1 | Psmb5 | Gm15533 | Mgp | Lockd |
| Phex | Sept4 | Mfsd2b | Igfbp7 | Ska1 |

TABLE 14
List of the genes utilized to identify the cell subclusters
within the osteogenic cells cluster of the mechanically EXPANDED
calvarial sutures of 2 month old mice. The 25 most significantly
expressed genes are reported for each cluster.

| | Osteoblasts |
|---|---|
| Progenitor cells | precursors |
| Lrrc15 | Slc4a10 |
| Tnn | lgf2 |
| Postn | Smoc1 |
| Gpx3 | Npr3 |
| Tnc | Atp1b1 |
| Nrp2 | Foxd1 |
| Srpx2 | S100b |
| En1 | Palmd |
| Col5a2 | Cpe |
| Capn6 | Igfbp2 |
| Meis2 | Sned1 |
| Col5a1 | Rgs7bp |
| C1qtnf3 | Ildr2 |
| Crispld2 | Prdm6 |
| Cdh2 | Foxc2 |
| Myo1b | Pfn2 |
| Lamb1 | Foxp2 |
| Angptl2 | Slc26a7 |
| Ccn4 | Slit2 |
| Tcfl5 | Apoe |
| Ncam1 | Shisa3 |
| Nnmt | Mdk |
| Crip1 | Ccn3 |
| Spp1 | Emb |
| Emp1 | Rbp1 |

### Mouse Suture Expansion Surgery and Creation of Calvarial Bone Defects.

Mice were anesthetized, a surgical incision was performed to expose the calvarial bones, and the expansion devise was applied. Then, the incision was closed to fully cover the expansion devise (FIGS. 18A-18B). Control mock surgery (non-expanded group) replicated every step of the surgical procedure, but expansion devise was not inserted.

For the bone regeneration studies, immediately after insertion of the suture expander, a defect of a diameter of 2.0 mm was manually created in the left parietal bone.

Mice were anesthetized by intraperitoneal injection of ketamine (100 mg/Kg) and xylazine (10 mg/Kg), and a longitudinal incision was made with a #15 scalpel blade over the midline of the calvarial skin to expose the parietal bones. Two equidistant holes, one in each parietal bone, were manually created 2 mm from the sagittal suture using a 0.25 mm round dental bur. Next, the expansion apparatus, made by bending a 0.3 mm (diameter) orthodontic nickel-titanium wire was inserted in the holes (FIG. 3A). Using a dial tension gauge (Teclock, Nagano, Japan) as previously described (34) the wire was calibrated to exert an initial tensile force of 0.2N. Then, using absorbable 5-0 Vicryl sutures (Hu-Friedy, Chicago, IL, United States), the incision was closed, fully covering the expansion devise. Control mock surgery (non-expanded group) consisted in the creation of the 2 equidistant holes in the parietal bones without the insertion of the expansion apparatus. Immediately after surgery, and every 12 h for the following 48 h, animals were injected with Buprenorphine (subcutaneous injection, 0.05 mg/kg) for pain control.

For the bone regeneration studies, immediately after insertion of the suture expander, a defect of a diameter of 2.0 mm was manually created in the left parietal bone using successive series of round dental burs (from 0.25 mm to 2 mm), at a distance of 3 mm from the sagittal suture and 1 mm from the lambdoid suture. Using absorbable 5-0 Vicryl sutures (Hu-Friedy, Chicago, IL, United States), the incision was closed to fully cover the expansion devise (FIGS. 18A-18B).

The distance of 3 mm from the expanding sagittal suture and of 1 mm from the lambdoid suture was chosen for two reasons. First, to limit the contribution of the pre-existing Prx1+ cells to the healing of the bone defect, since it has been reported that the distance of the bone defects from the calvarial sutures inversely influences the regeneration capacity, with a e-CSD distant 1 mm from the sagittal suture able to regenerate up to ˜50% of the missing bone and a e-CSD distant 2 mm from the suture able to regenerate only ˜20% of the missing bone (26). Second, to allow the insertion of the expansion devise along the mesio-distal axis of the sagittal suture, in a position sufficiently central to maximize the expansion of the entire suture and sufficiently distant from the created defect to avoid invasion of the devise into the regeneration site during healing.

### In Vivo Imaging and Quantification of Mouse Prx1/Prrx1 Expressing Cells.

Intravital microscopy and Prx1-creER-EGFP^{+/-} transgenic male mice were utilized for in vivo imaging and in vivo quantification of green fluorescent Prx1/Prrx1 expressing cells according to a methodology previously described (13,64). Two groups of mice, the expanded group and the non-expanded control group, were evaluated. Briefly, the excitation beam was focused into the sample plane using a 60X objective lens. Fluorescence emission was collected by two-photon detectors. EGFP signals were detected with a 525/38 band pass filter using two photon acquisition excited by the Ti: Sapphire laser pulsing at 900 nm (Mai tai, Spectra-Physics). Bone was visualized using second harmonic generation (SHG) of collagen and collected by a 435/40 nm band pass filter. Cell distribution was visualized in stack images at 3 μm intervals. To quantify the number of cells in the sutures, three equidistant regions across the sagittal suture of each animal were imaged. Cell counting was obtained throughout stack images representing 90 μm depth from the periosteal surface.

### In Situ Hybridization in Mouse Sagittal Sutures

To perform in situ hybridization, the RNAscope Multiplex Fluorescent Reagent Kit V2 (320850, RNAscope®, Advanced Cell Diagnostics, Inc., Newark, CA) was used according to manufacturer's recommendations.

Calvaria were harvested from Prx1-creER-EGFP^{+/-} male mice 2 days after surgery, fixed in 4% methanol-free paraformaldehyde, and decalcified using Morse's solution (63). The RNAscope Multiplex Fluorescent Reagent Kit V2 (320850, RNAscope®, Advanced Cell Diagnostics, Inc., Newark, CA) was used according to manufacturer's recommendations: 10 μm frozen sections were stained with positive control probes (320881), negative control probes (320871), and probes for targeting EGFP (400281, EGFP-AF488) and mouse ki67 (416771-C3, Mm-mki67 AF647). Fluorescence images are projected from the full tissue thickness (10 μm) by acquiring stack images at 1 μm intervals using an Olympus confocal microscope (FV1000), with a 40X objective lens (NA=0.8).

qPCR of Mouse Prx1/Prrx1 Expressing Cells

EGFP+ cells were sorted (average of 100-150 cells/animal) and gene expression analyses were performed using the Single Cell to CT kit (Thermo Fisher Scientific, City and State).

Prx1-creER-EGFP+i-male mice were euthanized and EGFP+ cells from the sagittal sutures were isolated using FACS. Briefly, sagittal sutures were manually dissected, pooled, and digested at 37° C. in a shaking water bath in two steps. In the first step, 3 mg/mL of collagenase II (Worthington Biochemical Inc., Lake Wood, NJ) in αMEM and 1% penicillin/streptomycin was utilized for 120 minutes; in the second step, 0.76 U/mL of collagenase P (Roche; Mannheim; Germany) and 0.67 U/mL of dispase (Worthington Biochemical; Inc., Lake Wood, NJ) in αMEM, and 1% penicillin/streptomycin were utilized for 60 minutes. EGFP+ cells were sorted (average of 100-150 cells/animal) and gene expression analyses were performed using the Single Cell to CT kit (Thermo Fisher Scientific, City and State). qPCR reactions were carried out using the TaqMan gene expression analysis kit. The following TaqMan assay codes were utilized: Actb (Mm02619580_g1), Axin2 (Mm00443610_ml), Birc5 (Mm01261895_ml), Ccnd](Mm00432359_ml), Ctnnb1 (Mm00483039_ml), Esp11 (Mm01299687_ml), Gapdh (Mm99999915_g1), Mki67 (Mm01278617_m), Tbp (Mm00446971_ml), and Tubb (Mm00495806_g). The geometrical average of the Ct values obtained for the Actb, Gapdh, Tbp, and Tubb housekeeping genes was utilized for relative quantifications.

### Micro-CT Analyses of Mouse Cranium

Mouse skulls were scanned using a Scanco μCT40 scanner (Scanco Medical AG, Basserdorf, Switzerland) at a voxel size of 20×20 X 20 μm, 70 kVp X-ray voltage, 114 mA intensity and 200 ms integration time. To remove noise, a low pass Gaussian filter of a width of 0.8 pixels and support of 1.0 was applied. Bone segmentation was conducted at a threshold of 300 (scale: 0-1,000) and the volume of interest (VOI) investigated included the 2 mm segmental defect and additional 0.5 mm in the peripheral regions. A semi-automated segmentation of cross-sectional tomograms was performed. Any reactionary calcification was excluded from the quantification. A global threshold of 161 (1793 HU, 300.0 mg HA/cm3) was applied based on histograms of the grey scale values of the segmented VOL. Within the VOI, the following parameters were analyzed: the segmented bone volume BV and the ratio of the segmented bone volume to the total volume (BV/TV, in %).

### Inducible Inactivation of Canonical Wnt Signaling in Mouse Prx1/Prrx1 Expressing Cells

Prx1-creER-EGFP^{+/-}; β-catenin^{+/+} mice and Prx1-creER-EGFP^{+/-}; β-catenin^{ﬂ/ﬂ} mice (identified in the figures as Prx1-creER-EGFP^{+/-};β-catenin^{-/-} mice to indicate the cre recombinase inactivation of the D-catenin gene) were injected with tamoxifen (intraperitoneally, 40 mg/Kg in sterile corn oil) 5 days before and 5 days after surgery. Sixty days after surgery, calvarial samples were fixed in 70% ethanol, subjected to μCT imaging, and analyzed. Histological assessment was performed following a decalcification step using Morse's solution.

### In Situ Hybridization of Human Sagittal Sutures

De-identified specimen of the human fetal calvarial tissue (age 80 days post-conception) were obtained from the Birth Defects Research Laboratory at the University of Washington with maternal written consent, in accordance with ethical and legal guidelines of the University of Washington (UW-IRB #380) and Seattle Children's Research Institute's (SCH-IRB #10925) institutional review boards. The RNAscope Multiplex Fluorescent Reagent Kit V2 (320850, RNAscope®, Advanced Cell Diagnostics, Inc., Newark, CA) was used according to manufacturer's recommendations. The calvaria sample was fixed and decalcified prior sectioning. The RNAscope Multiplex Fluorescent Reagent Kit V2 (320850, RNAscope®, Advanced Cell Diagnostics, Inc., Newark, CA) was used according to manufacturer's recommendations: 10 μm frozen sections were stained with positive control probes (UBC-AF647), negative control probes (DapB AF647), and probes for targeting Prx1 (hPrrx1-AF647). Fluorescence images are projected from the full tissue thickness (10 m) by acquiring stack images at 1 μm intervals using an Olympus confocal microscope (FV1000), with a 40X objective lens (NA=0.8).

qPCR of Human Sagittal Suture Cells

De-identified specimens from human fetal calvarial tissue (age 79-108 days post-conception) were obtained from the Birth Defects Research Laboratory at the University of Washington. Immediately after collection, the parietal bone tissue or the sagittal suture tissue was dissected and cells were isolated as previously described (64). After culturing, cells were dissociated from respective plates and RNA was immediately isolated with the High Pure miRNA Isolation Kit (Roche, Basel, Switzerland) per manufacturer instructions. qPCR reactions were carried out using the TaqMan gene expression analysis kit. The following TaqMan assay codes were utilized: Actb (Hs01060665_g1), Prx1/Prrx1 (4331182 Hs00246567_ml), Tbp (Hs00427620_ml), and Tubb (Hs00742828_g1).

The geometrical average of the Ct values obtained for the Actb, Tbp, and Tubb housekeeping genes was utilized for relative quantifications.

Statistical Analyses of μCT and qPCR Data

Student's t-test was utilized to identify statistically significant difference among the analyzed groups of mice or genes.

### REFERENCES

1. Fearon J A, Griner D, Ditthakasem K, Herbert M. Autogenous bone reconstruction of large secondary skull defects. Plast Reconstr Surg. 2017 February;139(2):427-38.
2. Abu-Ghname A, Banuelos J, Oliver J D, Vyas K, Daniels D, Sharaf B. Outcomes and complications of pediatric cranioplasty: A systematic review. Plast Reconstr Surg. 2019 Sep;144(3):433e-43e.
3. van de Vijfeijken S E C M, Groot C, Ubbink D T, Vandertop W P, Depauw P R A M, Nout E, et al. Factors related to failure of autologous cranial reconstructions after decompressive craniectomy. J Craniomaxillofac Surg. 2019 September;47(9):1420-5.
4. Czerwinski M, Hopper R A, Gruss J, Fearon J A. Major morbidity and mortality rates in craniofacial surgery: an analysis of 8101 major procedures. Plast Reconstr Surg. 2010 Jul;126(1):181-6.
5. Tevlin R, McArdle A, Atashroo D, Walmsley GG, Senarath-Yapa K, Zielins E R, et al. Biomaterials for craniofacial bone engineering. J Dent Res. 2014 December; 93(12):1187-95.
6. Woo E J. Adverse events reported after the use of recombinant human bone morphogenetic protein 2. J Oral Maxillofac Surg. 2012 April; 70(4):765-7.
7. Mesfin A, Buchowski J M, Zebala L P, Bakhsh W R, Aronson A B, Fogelson J L, et al. High-dose rhBMP-2 for adults: major and minor complications: a study of 502 spine cases. J Bone Joint Surg Am. 2013 Sep. 4; 95(17):1546-53.
8. Zuk P A. Tissue engineering craniofacial defects with adult stem cells? Are we ready yet?Pediatr Res. 2008 May; 63(5):478-86.
9. Marra K G, Rubin J P. The potential of adipose-derived stem cells in craniofacial repair and regeneration. Birth Defects Res C Embryo Today. 2012 March;96(1):95-7.
10. Doro D H, Grigoriadis A E, Liu K J. Calvarial Suture-Derived Stem Cells and Their Contribution to Cranial Bone Repair. Front Physiol. 2017 Nov. 27; 8:956.
11. Martin J F, Olson E N. Identification of a prx1 limb enhancer. Genesis. 2000 Apr;26(4):225-9.
12. Logan M, Martin J F, Nagy A, Lobe C, Olson E N, Tabin C J. Expression of Cre Recombinase in the developing mouse limb bud driven by a Prx1 enhancer. Genesis. 2002 Jun;33(2):77-80.
13. Wilk K, Yeh S-C A, Mortensen L J, Ghaffarigarakani S, Lombardo C M, Bassir S H, et al. Postnatal Calvarial Skeletal Stem Cells Expressing PRX1 Reside Exclusively in the Calvarial Sutures and Are Required for Bone Regeneration. Stem Cell Reports. 2017 Apr. 11; 8(4):933-46.
14. Zhao H, Feng J, Ho T-V, Grimes W, Urata M, Chai Y. The suture provides a niche for mesenchymal stem cells of craniofacial bones. Nat Cell Biol. 2015 April; 17(4):386-96.
15. Maruyama T, Jeong J, Sheu T-J, Hsu W. Stem cells of the suture mesenchyme in craniofacial bone development, repair and regeneration. Nat Commun. 2016 Feb. 1; 7:10526.
16. Debnath S, Yallowitz A R, McCormick J, Lalani S, Zhang T, Xu R, et al. Discovery of a periosteal stem cell mediating intramembranous bone formation. Nature. 2018 Oct;562(7725):133-9.
17. Aalami O O, Nacamuli R P, Lenton K A, Cowan C M, Fang T D, Fong K D, et al. Applications of a mouse model of calvarial healing: differences in regenerative abilities of juveniles and adults. Plast Reconstr Surg. 2004 Sep. 1; 114(3):713-20.
18. Sirola K. Regeneration of defects in the calvaria. An experimental study. Ann Med Exp Biol Fenn. 1960;38(Suppl 2):1-87.
19. Pritchard J J, Scott J H, Girgis F G. The structure and development of cranial and facial sutures. J Anat. 1956 January;90(1):73-86.
20. Morriss-Kay G M, Wilkie A O M. Growth of the normal skull vault and its alteration in craniosynostosis: insights from human genetics and experimental studies. J Anat. 2005 Nov;207(5):637-53.
21. Grova M, Lo D D, Montoro D, Hyun J S, Chung M T, Wan D C, et al. Models of cranial suture biology. J Craniofac Surg. 2012 November;23(7 Suppl 1):1954-8.
22. Ignelzi M A, Liu Y H, Maxson R E, Snead M L. Genetically engineered mice: tools to understand craniofacial development. Crit Rev Oral Biol Med. 1995; 6(3):181-201.
23. Holmes G. The role of vertebrate models in understanding craniosynostosis. Childs Nerv Syst. 2012 September;28(9):1471-81.
24. Menon S, Salhotra A, Shailendra S, Tevlin R, Ransom R C, Januszyk M, et al. Skeletal stem and progenitor cells maintain cranial suture patency and prevent craniosynostosis. Nat Commun. 2021 Jul. 30; 12(1):4640.
25. Murphy M P, Quarto N, Longaker M T, Wan D C. * Calvarial Defects: Cell-Based Reconstructive Strategies in the Murine Model. Tissue Eng Part C Methods. 2017 Dec;23(12):971-81.
26. Park S, Zhao H, Urata M, Chai Y. Sutures possess strong regenerative capacity for calvarial bone injury. Stem Cells Dev. 2016 Dec. 1; 25(23):1801-7.
27. Lo Celso C, Fleming H E, Wu J W, Zhao C X, Miake-Lye S, Fujisaki J, et al. Live-animal tracking of individual haematopoietic stem/progenitor cells in their niche. Nature. 2009 Jan. 1; 457(7225):92-6.
28. Yeh S-C A, Wilk K, Lin C P, Intini G. In vivo 3D histomorphometry quantifies bone apposition and skeletal progenitor cell differentiation. Sci Rep. 2018 Apr. 3; 8(1):5580.
29. Marghoub A, Libby J, Babbs C, Ventikos Y, Fagan M J, Moazen M. Characterizing and Modeling Bone Formation during Mouse Calvarial Development. Phys Rev Lett. 2019 Feb. 1; 122(4):048103.
30. Scholzen T, Gerdes J. The Ki-67 protein: from the known and the unknown. J Cell Physiol. 2000 March;182(3):311-22.
31. Matsuura I, Denissova N G, Wang G, He D, Long J, Liu F. Cyclin-dependent kinases regulate the antiproliferative function of Smads. Nature. 2004 Jul. 8; 430(6996):226-31.
32. Hauf S, Waizenegger I C, Peters J M. Cohesin cleavage by separase required for anaphase and cytokinesis in human cells. Science. 2001 Aug. 17; 293(5533):1320-3.
33. Li F, Ambrosini G, Chu E Y, Plescia J, Tognin S, Marchisio P C, et al. Control of apoptosis and mitotic spindle checkpoint by survivin. Nature. 1998 Dec. 10; 396(6711):580-4.
34. Takeshita N, Hasegawa M, Sasaki K, Seki D, Seiryu M, Miyashita S, et al. In vivo expression and regulation of genes associated with vascularization during early response of sutures to tensile force. J Bone Miner Metab. 2017 January;35(1):40-51.
35. Kawanami A, Matsushita T, Chan Y Y, Murakami S. Mice expressing GFP and CreER in osteochondro progenitor cells in the periosteum. Biochem Biophys Res Commun. 2009 Aug. 28; 386(3):477-82.
36. Rindone A N, Liu X, Farhat S, Perdomo-Pantoja A, Witham T F, Coutu D L, et al. Quantitative 3D imaging of the cranial microvascular environment at single-cell resolution. Nat Commun. 2021 Oct. 28; 12(1):6219.
37. Lam F, Morris C. Nine year old boy with chromosome 1q23.3-q25.1 deletion. Am J Med Genet A. 2016 November; 170(11):3013-7.
38. Sergi C, Kamnasaran D. PRRX1 is mutated in a fetus with agnathia-otocephaly. Clin Genet. 2011 March;79(3):293-5.
39. Timberlake A T, Kiziltug E, Jin S C, Nelson-Williams C, Loring E, Yale Center for Genome Analysis, et al. De novo mutations in the BMP signaling pathway in lambdoid craniosynostosis. Hum Genet. 2022 Aug. 23;
40. Borrelli M R, Hu M S, Longaker M T, Lorenz H P. Tissue engineering and regenerative medicine in craniofacial reconstruction and facial aesthetics. J Craniofac Surg. 2020; 31(1):15-27.
41. Mammoto T, Mammoto A, Ingber D E. Mechanobiology and developmental control. Annu Rev Cell Dev Biol. 2013; 29:27-61.
42. Kolpakova-Hart E, McBratney-Owen B, Hou B, Fukai N, Nicolae C, Zhou J, et al. Growth of cranial synchondroses and sutures requires polycystin-1. Dev Biol. 2008 Sep. 15; 321(2):407-19.
43. Khonsari R H, Olivier J, Vigneaux P, Sanchez S, Tafforeau P, Ahlberg P E, et al. A mathematical model for mechanotransduction at the early steps of suture formation. Proc Biol Sci. 2013 May 22;280(1759):20122670.
44. Hoey D A, Kelly D J, Jacobs C R. A role for the primary cilium in paracrine signaling between mechanically stimulated osteocytes and mesenchymal stem cells. Biochem Biophys Res Commun. 2011 Aug. 19; 412(1):182-7.
45. Ransom R C, Carter A C, Salhotra A, Leavitt T, Marecic O, Murphy M P, et al. Mechanoresponsive stem cells acquire neural crest fate in jaw regeneration. Nature. 2018 Nov;563(7732):514-21.
46. Moore E R, Chen J C, Jacobs C R. Prx1-Expressing Progenitor Primary Cilia Mediate Bone Formation in response to Mechanical Loading in Mice. Stem Cells Int. 2019 Nov. 11; 2019:3094154.
47. Ikegame M, Tabuchi Y, Furusawa Y, Kawai M, Hattori A, Kondo T, et al. Tensile stress stimulates the expression of osteogenic cytokines/growth factors and matricellular proteins in the mouse cranial suture at the site of osteoblast differentiation. Biomed Res. 2016; 37(2):117-26.
48. Ikegame M, Ejiri S, Okamura H. Expression of Non-collagenous Bone Matrix Proteins in Osteoblasts Stimulated by Mechanical Stretching in the Cranial Suture of Neonatal Mice. J Histochem Cytochem. 2019 February;67(2):107-16.
49. Mani P, Jarrell A, Myers J, Atit R. Visualizing canonical Wnt signaling during mouse craniofacial development. Dev Dyn. 2010 January;239(1):354-63.
50. Mishina Y, Snider T N. Neural crest cell signaling pathways critical to cranial bone development and pathology. Exp Cell Res. 2014 Jul. 15; 325(2):138-47.
51. Minear S, Leucht P, Jiang J, Liu B, Zeng A, Fuerer C, et al. Wnt proteins promote bone regeneration. Sci Transl Med. 2010 Apr. 28; 2(29):29ra30.
52. Fu H-D, Wang B-K, Wan Z-Q, Lin H, Chang M-L, Han G-L. Wnt5a mediated canonical Wnt signaling pathway activation in orthodontic tooth movement: possible role in the tension force-induced bone formation. J Mol Histol. 2016 October;47(5):455-66.
53. Wang X, Luo E, Bi R, Ye B, Hu J, Zou S. Wnt/p-catenin signaling is required for distraction osteogenesis in rats. Connect Tissue Res. 2018 January;59(1):45-54.
54. Donnelly M, Todd E, Wheeler M, Winn V D, Kamnasaran D. Prenatal diagnosis and identification of heterozygous frameshift mutation in PRRX1 in an infant with agnathia-otocephaly. Prenat Diagn. 2012 September;32(9):903-5.
55. Winters R, Tatum S A. Craniofacial distraction osteogenesis. Facial Plast Surg Clin North Am. 2014 Nov. 8; 22(4):653-64.
56. McCarthy J G, Warren S M, Bernstein J, Burnett W, Cunningham ML, Edmond J C, et al. Parameters of care for craniosynostosis. Cleft Palate Craniofac J. 2012 January;49 Suppl:lS-24S.
57. Burstein F D, Williams J K, Hudgins R, Graham L, Teague G, Paschal M, et al. Single-stage craniofacial distraction using resorbable devices. J Craniofac Surg. 2002 Nov;13(6):776-82.
58. Dutta S, Sengupta P. Men and mice: Relating their ages. Life Sci. 2016 May 1; 152:244-8.
59. Duchamp de Lageneste O, Julien A, Abou-Khalil R, Frangi G, Carvalho C, Cagnard N, et al. Periosteum contains skeletal stem cells with high bone regenerative potential controlled by Periostin. Nat Commun. 2018 Feb. 22; 9(1):773.
60. Esposito A, Wang L, Li T, Miranda M, Spagnoli A. Role of Prx1-expressing skeletal cells and Prx1-expression in fracture repair. Bone. 2020 October;139:115521.
61. Holmes G, Gonzalez-Reiche A S, Lu N, Zhou X, Rivera J, Kriti D, et al. Integrated transcriptome and network analysis reveals spatiotemporal dynamics of calvarial suturogenesis. Cell Rep. 2020 Jul. 7; 32(1):107871.
62. Farmer D T, Mlcochova H, Zhou Y, Koelling N, Wang G, Ashley N, et al. The developing mouse coronal suture at single-cell resolution. Nat Commun. 2021 Aug. 10; 12(1):4797.
63. de Charleroy C, Haseeb A, Lefebvre V. Preparation of adult mouse skeletal tissue sections for RNA in situ hybridization. Methods Mol Biol. 2021; 2245:85-92.
64. Homayounfar N, Park S S, Afsharinejad Z, Bammler T K, MacDonald J W, Farin F M, et al. Transcriptional analysis of human cranial compartments with different embryonic origins. Arch Oral Biol. 2015 September;60(9):1450-60.

### Example 2

One significant barrier separates the use of skeletal stem cells (SSCs) from successful therapies: the lack of strategies to endogenously harness them. These “endogenous strategies”, by circumventing the significant biological, regulatory, and financial barriers that are associated to the current therapeutic protocols based on the ex vivo expansion of stem cells, may represent a significant therapeutic advance (1,2). Over the past several years it was shown that, at least in mice, expression of PRX1 (or PRRX1), a transcription factor highly expressed during limb and craniofacial development (3,4), identifies a population of post-natal skeletal stem cells of the calvarial bone that is responsible and required for the calvarial bone regeneration process (5,6). Also, it was shown that the number of these cells declines as mice age (5). Additional compelling studies by other investigators have independently confirmed that expression of PRX1 also identifies SSCs of the long bones and that these cells are involved with bone regeneration (7-15). Therefore, methods have been investigated that, by inducing a proliferation of the PRX1 expressing SSCs, can harness these cells to induce regeneration of skeletal tissues and to rejuvenate the skeleton. One such method is based on the mechanical stimulation of the SSCs within the calvarial bone (see current studies, below). The data shows that such stimulation, in the form of a controlled expansion of the calvarial sagittal sutures where the SSCs reside, induces proliferation of the PRX1 expressing SSCs and can, consequently, sustain endogenous bone regeneration without requiring implantation of biomaterials (16). This process resembles the spontaneous endogenous calvarial bone regeneration that is observed in children up to 2 years old, during their early stages of craniofacial development and calvarial suture expansion (17,18). Altogether, this data confirms that PRX1 expressing SSCs can be successfully harnessed to sustain bone regeneration and rejuvenate the calvarial bones. Thus, next it was investigated whether any small molecule could induce proliferation of the PRX1 expressing SSCs. Niclosamide can induce proliferation of PRX1 expressing SSCs in vitro, ex vivo, and in vivo (see current studies, below), and therefore it is proposed that Niclosamide is an agent that can be used to harness SSCs to sustain bone regeneration and to rejuvenate skeletal tissues.

### Mouse Skeletal Stem Cells (SSCs) Expressing PRX1 can be Harnessed to Induce

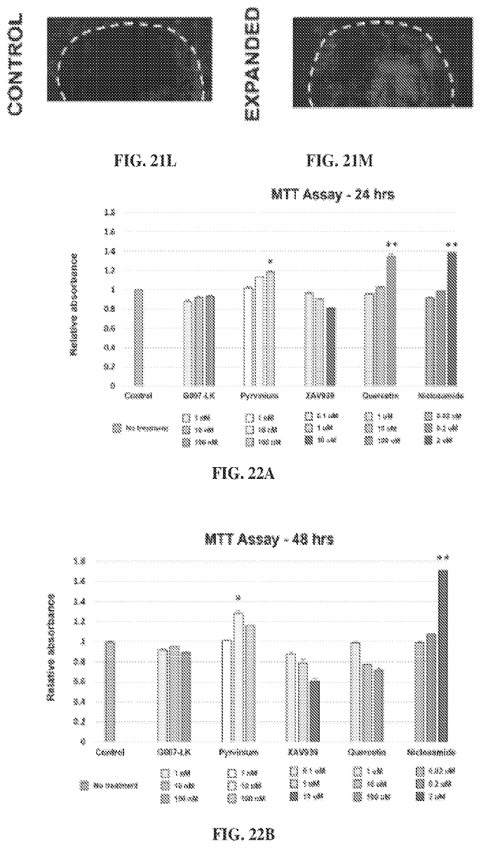

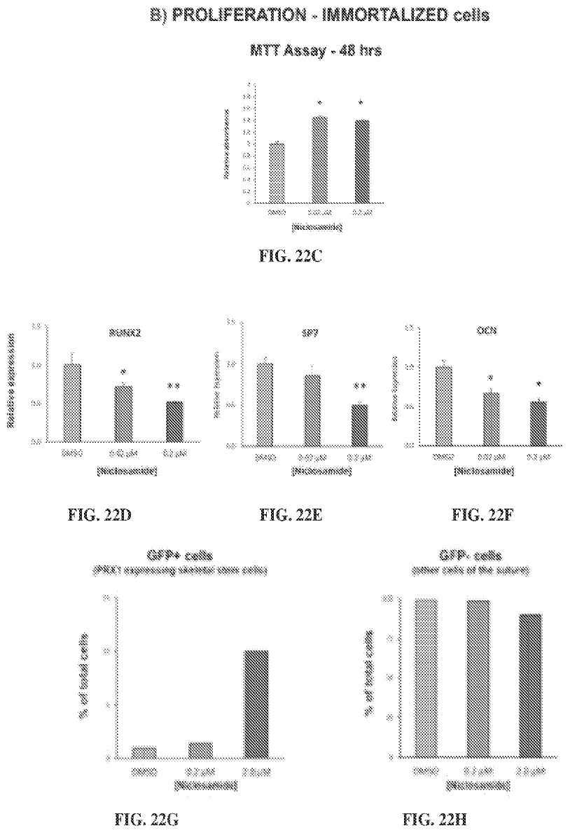

Endogenous Bone Regeneration. Novel strategies to harness stem cells and endogenously induce tissue regeneration have been researched. Having identified PRX1 expressing cells of the calvarial sutures as skeletal stem cells (SSCs)(5), it was investigated whether a strategy can be found to harness these cells to stimulate endogenous bone regeneration. Studies indicate that in children up to 2 years of age, regeneration of calvarial defects occurs naturally and without therapeutic aids 17,18. After two years of age, this regeneration capacity is lost (17,18), coinciding with a significant reduction of the calvarial growth rate and with the closure of the calvarial sutures into mature synarthroses (19). This aging-associated loss of bone regeneration competency is observed in mice as well, as juvenile 6-day old mice can regenerate critical size defects (CSD, defects that do not spontaneously heal) in parietal bones, whereas skeletally mature mice cannot (17). Since the bone regeneration competency coincides, in terms of time, with the presence of open/not matured sutures (19-21), it was hypothesized that the open sutures represent a reservoir of skeletal stem cells that can contribute to bone regeneration. Thus, using scRNA-seq it was first shown that PRX1 expressing cells are present in the sutures of young 4-day old mice but absent in the sutures of skeletally mature mice (FIGS. 21A-21B). Then, a controlled mechanical expansion of the sagittal suture of skeletally mature 8-week old mice was shown, by mimicking the expansion of the sutures that occurs during the skull development (19), induces proliferation of the PRX1 expressing SSCs (FIGS. 21D-21J) and sustains full regeneration of a 2 mm in diameter CSD (otherwise unable to heal (22)), created simultaneously to the expansion of the sutures (FIG. 21K). A lineage tracing analysis confirms that the regeneration occurs by means of PRX1 expressing SSCs' progeny (FIGS. 21L-21M). All together, these studies indicate that an “activation” (by means of a mechanical expansion) of the suture is able to induce proliferation of the PRX1 expressing SSCs of the sutures and sustains full regeneration of the calvarial CSD. Significantly, this regeneration occurs without the aid of any biomaterial inserted into the bone defect. Thus, the mechanical expansion of the suture represents an effective harnessing strategy of the PRX1 expressing SSCs to sustain endogenous bone regeneration of the calvaria, de facto mimicking what can happen in the calvaria of children up to 2 years old.

### Niclosamide Induces Proliferation of the Mouse PRX1 Expressing SSCs.

It is well documented that canonical WNT signaling is significantly active during craniofacial bone formation (23) and regulates osteoblastogenesis (24). It was shown, that both in vivo and in vitro, that PRX1 expressing cells of the calvarial suture respond to an agonist of WNT signaling by differentiating into osteoblastic-like cells (5,6). Therefore, next it was tested whether inhibition of WNT signaling, by reversing differentiation of PRX1 expressing cells, could induce proliferation instead. Several evidences support this hypothesis. First, studies by others have shown that, at least for tissues like heart, cartilage, and lung, a temporary inhibition of canonical WNT signaling increases the number of progenitor cells and promotes wound regeneration (25-27). Second, it has been shown that DKK1, a well-studied inhibitor of canonical WNT signaling, is able to induce proliferation of bone marrow-derived stem cells (28). Third, it has been also shown that a temporal inhibition of WNT signaling during the early stages of fracture repair (induced by means of an adenovirus-mediated over-expression of DKK1) improves healing of the bone fracture (29). Finally, preliminary data shown in FIG. 21A-21J also indicate that DKK1 is upregulated during the mechanically-induced proliferation of PRX1 expressing cells of the calvarial sutures (FIG. 21H).

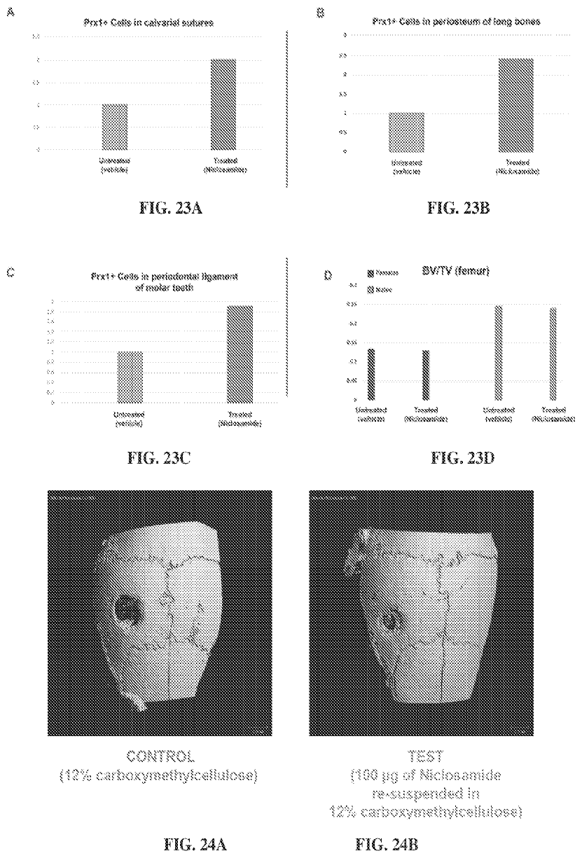

WNT inhibitor candidates were identified by looking into cancer studies, as thousands of small molecules have been screened for their potential to inhibit WNT signaling in WNT-activated cancer cells (30). Several molecules have shown inhibition of canonical WNT activity in vitro but only few have been tested in vivo, in mice. Among those tested in vivo, 5 were selected because of their proven efficacy and safety: G007-LK (31), Pyrvinium (25,26), Quercetin (32,33), XAV939 (27), and Niclosamide (34). Actually, Niclosamide and Pyrvinium are anti-helminthic FDA-approved drugs (35,36) and Quercetin is a flavonol found in many fruits and vegetables that is used as an ingredient in supplements, beverages, or foods (33). First, the 5 molecules were screened in vitro for their ability to induce proliferation of primary PRX1 expressing SSCs isolated from the calvarial sutures of PRX1-creER-EGFP mice. the most effective concentrations used in the previously mentioned studies (FIG. 22A) were selected and tested. Pyrvinium, Quercetin, and Niclosamide were able to consistently induce proliferation, with Niclosamide producing the most significant and prolonged effect (FIGS. 22A-22B). Then, Niclosamide was confirmed that could induce proliferation of immortalized PRX1 expressing SSCs as well (FIG. 22C). As also reported by others (37), a quantitative RT-PCR also confirmed that Niclosamide, while inducing proliferation of the PRX1 expressing SSCs, also inhibits their osteoblastic differentiation (FIGS. 22D-22F). Importantly, proliferation of the PRX1 expressing SSCs was observed even ex vivo, when explanted whole sutures were exposed to Niclosamide (FIGS. 22G-22H). Such effect could not be detected in the other cells of the sutures, indicating that Niclosamide might have a PRX1 expressing cells-specific effect (FIGS. 22G-22H). All together this data indicates that Niclosamide effectively induces proliferation of PRX1 expressing SSCs, and it does so more effectively than Pyrvinium and Quercetin. Previous studies indicate that Niclosamide, besides inhibiting the WNT pathway, presents with various additional effects such as uncoupling oxidative phosphorylation and disrupting several different signaling pathways, including the NOTCH, mTORCT, NFkB, and STAT3 pathways (38-44). Therefore, the observed superior effectiveness of Niclosamide over Pyrvinium and Quercetin is related to its multiple biological activities.

Having observed that Niclosamide can induce proliferation of PRX1 expressing SSCs in vitro and ex vivo, next it was confirmed that Niclosamide can effectively induce proliferation of PRX1 expressing SSCs in vivo. To do so, skeletally mature 8-week old mice were treated with intraperitoneal (IP) injections of Niclosamide (20 mg/Kg of body weight) or vehicle (control) 5 days/week, for 3 weeks (FIGS. 23A-23D). The obtained data indicates that treatment with Niclosamide induces proliferation of PRX1 expressing SSCs in the calvarial sutures (FIG. 23A), in the periosteum of long bones (FIG. 23B), and in the periodontal ligament of the mouse molars (FIG. 23C). Importantly, the 3-week treatment showed that it does not significantly reduce the bone mass in both males and female mice (FIG. 23D), suggesting that, despite its WNT inhibitory effects and related reduction of osteoblastogenesis (FIGS. 22D-22F and (37)), Niclosamide, at least at this regimen, does not alter the bone mass of the long bones. This lack of negative effect on the bone mass of the long bones should be seen as a significant therapeutic advantage as it would allow Niclosamide to be administered for short periods of time during the life span of an individual, without compromising the individual's bone mass. Thus, Niclosamide treatment could sustain bone regeneration and bone rejuvenate without compromising bone health.

### CONCLUSION

By providing this information about Niclosamide and its ability to induce proliferation of SSCs, Niclosamide represents a novel bone regeneration and bone rejuvenation therapeutic.

In vivo experiments can be performed to test: 1) Niclosamide is effectiveness when locally delivered (i.e. subcutaneously, by the calvarial sutures; 2) Niclosamide's induced proliferation of SSCs to sustain bone regeneration (recapitulating the in vivo studies shown in FIGS. 22A-22H, using Niclosamide); and 3) short term treatments of Niclosamide, over the life span of a mouse, to preserve their bone mass and their ability to heal bone fractures during aging or after ovarectomy.

### REFERENCES

1. Jenkins M J, Farid S S. Human pluripotent stem cell-derived products: advances towards robust, scalable and cost-effective manufacturing strategies. Biotechnol J. 2015 Jan;10(1):83-95.
2. Hassan S, Huang H, Warren K, Mahdavi B, Smith D, Jong S, et al. Process change evaluation framework for allogeneic cell therapies: impact on drug development and commercialization. Regen Med. 2016 April; 11(3):287-305.
3. Martin J F, Olson E N. Identification of a prx1 limb enhancer. Genesis. 2000 Apr;26(4):225-9.
4. Logan M, Martin J F, Nagy A, Lobe C, Olson E N, Tabin C J. Expression of Cre Recombinase in the developing mouse limb bud driven by a Prx1 enhancer. Genesis. 2002 Jun;33(2):77-80.
5. Wilk K, Yeh S-CA, Mortensen L J, Ghaffarigarakani S, Lombardo C M, Bassir S H, et al. Postnatal Calvarial Skeletal Stem Cells Expressing PRX1 Reside Exclusively in the Calvarial Sutures and Are Required for Bone Regeneration. Stem Cell Reports. 2017 Apr. 11; 8(4):933-46.
6. Yeh S-CA, Wilk K, Lin CP, Intini G. In vivo 3D histomorphometry quantifies bone apposition and skeletal progenitor cell differentiation. Sci Rep. 2018 Apr. 3; 8(1):5580.
7. Lu X, Beck J G R, Gilbert L C, Camalier C E, Bateman N W, Hood B L, et al. Identification of the homeobox protein Prx1 (MHox, Prrx-1) as a regulator of osterix expression and mediator of tumor necrosis factor i±action in osteoblast differentiation. J Bone Miner Res. 201t;26(1):209-19.
8. Du B, Cawthorn W P, Su A, Doucette C R, Yao Y, Hemati N, et al. The transcription factor paired-related homeobox 1 (Prrx1) inhibits adipogenesis by activating transforming growth factor-$beta$ (TGF$beta$) signaling. The Journal of biological chemistry. 2013;288(5):3036-3047.
9. Kawanami A, Matsushita T, Chan Y Y, Murakami S. Mice expressing GFP and CreER in osteochondro progenitor cells in the periosteum. Biochem Biophys Res Commun. 2009 Aug. 28; 386(3):477-82.
10. Ouyang Z, Chen Z, Ishikawa M, Yue X, Kawanami A, Leahy P, et al. Prx1 and 3.2kb Co11a1 promoters target distinct bone cell populations in transgenic mice. Bone. 2014 Jan;58:136-45.
11. Esposito A, Wang L, Li T, Miranda M, Spagnoli A. Role of Prx1-expressing skeletal cells and Prx1-expression in fracture repair. Bone. 2020 October;139:115521.
12. Julien A, Perrin S, Duchamp de Lageneste O, Carvalho C, Bensidhoum M, Legeai-Mallet L, et al. FGFR3 in Periosteal Cells Drives Cartilage-to-Bone Transformation in Bone Repair. Stem Cell Reports. 2020 Oct. 13; 15(4):955-67.
13. Duchamp de Lageneste 0, Julien A, Abou-Khalil R, Frangi G, Carvalho C, Cagnard N, et al. Periosteum contains skeletal stem cells with high bone regenerative potential controlled by Periostin. Nat Commun. 2018 Feb. 22; 9(1):773.
14. Ortinau L C, Wang H, Lei K, Deveza L, Jeong Y, Hara Y, et al. Identification of functionally distinct mxl+asma+periosteal skeletal stem cells. Cell Stem Cell. 2019 Dec. 5; 25(6):784-796.e5.
15. Bragdon B C, Bennie A, Molinelli A, Liu Y. Post natal expression of Prx1 labels appendicular restricted progenitor cell populations of multiple tissues. Journal of Cellular 2022;
16. Aldawood Z A, Mancinelli L, Geng X, Yeh S-CA, Gustafson J, Wilk K, et al. Expansion of the calvarial suture sustains endogenous regeneration of remote calvarial bone defects. Submitted (now in revision). 2021;
17. Aalami O O, Nacamuli R P, Lenton K A, Cowan C M, Fang T D, Fong K D, et al. Applications of a mouse model of calvarial healing: differences in regenerative abilities of juveniles and adults. Plast Reconstr Surg. 2004 Sep. 1; 114(3):713-20.
18. Sirola K. Regeneration of defects in the calvaria. An experimental study. Ann Med Exp Biol Fenn. 1960; 38(Suppl 2):1-87.
19. Cunningham M L, Heike C L. Evaluation of the infant with an abnormal skull shape. Curr Opin Pediatr. 2007 December; 19(6):645-51.
20. Vora S R, Camci E D, Cox T C. Postnatal Ontogeny of the Cranial Base and Craniofacial Skeleton in Male C57BL/6J Mice: A Reference Standard for Quantitative Analysis. Front Physiol. 2015; 6:417.
21. Wei X, Thomas N, Hatch N E, Hu M, Liu F. Postnatal craniofacial skeletal development of female c57bl/6ncrl mice. Front Physiol. 2017 Sep. 14; 8:697.
22. Cooper G M, Mooney M P, Gosain A K, Campbell P G, Losee J E, Huard J. Testing the critical size in calvarial bone defects: revisiting the concept of a critical-size defect. Plast Reconstr Surg. 2010 June;125(6):1685-92.
23. Mani P, Jarrell A, Myers J, Atit R. Visualizing canonical Wnt signaling during mouse craniofacial development. Dev Dyn. 2010 January;239(1):354-63.
24. Baron R, Kneissel M. WNT signaling in bone homeostasis and disease: from human mutations to treatments. Nat Med. 2013 Feb. 6; 19(2):179-92.
25. Saraswati S, Alfaro M P, Thorne C A, Atkinson J, Lee E, Young P P. Pyrvinium, a potent small molecule Wnt inhibitor, promotes wound repair and post-MI cardiac remodeling. PLoS ONE. 2010 Nov. 29; 5(11):e15521.
26. Bastakoty D, Saraswati S, Cates J, Lee E, Nanney L B, Young P P. Inhibition of Wnt/0-catenin pathway promotes regenerative repair of cutaneous and cartilage injury. FASEB J. 2015 Dec;29(12):4881-92.
27. Wang C, Zhu H, Sun Z, Xiang Z, Ge Y, Ni C, et al. Inhibition of Wnt/B-catenin signaling promotes epithelial differentiation of mesenchymal stem cells and repairs bleomycin-induced lung injury. Am J Physiol, Cell Physiol. 2014 Aug. 1; 307(3):C234-44.
28. Gregory C A, Singh H, Perry A S, Prockop D J. The Wnt signaling inhibitor dickkopf-1 is required for reentry into the cell cycle of human adult stem cells from bone marrow. J Biol Chem. 2003 Jul. 25; 278(30):28067-78.
29. Baht G S, Silkstone D, Vi L, Nadesan P, Amani Y, Whetstone H, et al. Exposure to a youthful circulaton rejuvenates bone repair through modulation of 0-catenin. Nat Commun. 2015 May 19; 6:7131.
30. Zhang X, Hao J. Development of anticancer agents targeting the Wnt/-catenin signaling. Am J Cancer Res. 2015 Jul. 15; 5(8):2344-60.
31. Lau T, Chan E, Callow M, Waaler J, Boggs J, Blake R A, et al. A novel tankyrase small-molecule inhibitor suppresses APC mutation-driven colorectal tumor growth. Cancer Res. 2013 May 15;73(10):3132-44.
32. Liang W, Luo Z, Ge S, Li M, Du J, Yang M, et al. Oral administration of quercetin inhibits bone loss in rat model of diabetic osteopenia. Eur J Pharmacol. 2011 Nov. 16; 670(1):317-24.
33. Park C H, Chang J Y, Hahm E R, Park S, Kim H-K, Yang C H. Quercetin, a potent inhibitor against beta-catenin/Tcf signaling in SW480 colon cancer cells. Biochem Biophys Res Commun. 2005 Mar. 4; 328(1):227-34.
34. Chen M, Wang J, Lu J, Bond MC, Ren X-R, Lyerly H K, et al. The anti-helminthic niclosamide inhibits Wnt/Frizzledl signaling. Biochemistry. 2009 Nov. 3; 48(43):10267-74.
35. Pawlowski Z S. Role of chemotherapy of taeniasis in prevention of neurocysticercosis. Parasitol Int. 2006;55 Suppl:S105-9.
36. Li B, Flaveny C A, Giambelli C, Fei D L, Han L, Hang B I, et al. Repurposing the FDA-approved pinworm drug pyrvinium as a novel chemotherapeutic agent for intestinal polyposis. PLoS ONE. 2014 Jul. 8; 9(7):e101969.
37. Liu F-L, Chen C-L, Lee C-C, Wu C-C, Hsu T-H, Tsai C-Y, et al. The Simultaneous Inhibitory Effect of Niclosamide on RANKL-Induced Osteoclast Formation and Osteoblast Differentiation. Int J Med Sci. 2017 Jul. 19; 14(9):840-52.
38. Pan J-X, Ding K, Wang C-Y. Niclosamide, an old antihelminthic agent, demonstrates antitumor activity by blocking multiple signaling pathways of cancer stem cells. Chin J Cancer. 2012 Apr;31(4):178-84.
39. Jin Y, Lu Z, Ding K, Li J, Du X, Chen C, et al. Antineoplastic mechanisms of niclosamide in acute myelogenous leukemia stem cells: inactivation of the NF-kappaB pathway and generation of reactive oxygen species. Cancer Res. 2010 Mar. 15; 70(6):2516-27.
40. Li Y, Li P-K, Roberts M J, Arend R C, Samant R S, Buchsbaum D J. Multi-targeted therapy of cancer by niclosamide: A new application for an old drug. Cancer Lett. 2014 Jul. 10; 349(1):8-14.
41. Suliman M A, Zhang Z, Na H, Ribeiro A L L, Zhang Y, Niang B, et al. Niclosamide inhibits colon cancer progression through downregulation of the Notch pathway and upregulation of the tumor suppressor miR-200 family. Int J Mol Med. 2016 September;38(3):776-84.
42. Li R, Hu Z, Sun S-Y, Chen ZG, Owonikoko T K, Sica G L, et al. Niclosamide overcomes acquired resistance to erlotinib through suppression of STAT3 in non-small cell lung cancer. Mol Cancer Ther. 2013 October;12(10):2200-12.
43. You S, Li R, Park D, Xie M, Sica G L, Cao Y, et al. Disruption of STAT3 by niclosamide reverses radioresistance of human lung cancer. Mol Cancer Ther. 2014 Mar;13(3):606-16.
44. Chen W, Mook R A, Premont R T, Wang J. Niclosamide: Beyond an antihelminthic drug. Cell Signal. 2018 January;41:89-96.

### Example 3. Local Delivery of Niclosamide

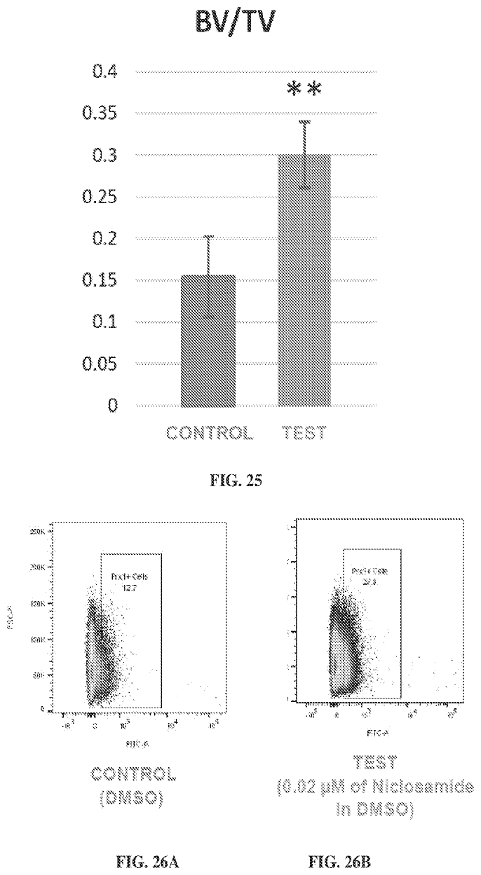

Local delivery of niclosamide sustains bone regeneration: To test that locally delivered niclosamide, by inducing a proliferation of the skeletal stem cells, can sustain bone regeneration, a critical size calvarialbone defect (2 mm in diameter) was created in the right parietal bone of skeletally mature C57B1/6 female mice (8 week oldmice). Immediately after creation of the defect, 10 μL of a 12% carboxymethylcellulose gel (CONTROL) or 10 μL of a 12% carboxymethylcellulose gel containing 100 g of niclosamide (TEST) were implanted within the bone defect (FIGS. 24A-24B). Bone regeneration was assessed by means of μCT, 4 weeks after surgery (FIG. 25). Student's t-test was used for statistical evaluation (n=3, ** indicates p<0.01).

This newly acquired data indicate that niclosamide can induce a substantial amount of bone regeneration when delivered locally, within a bone defect. A gel of the fast resorbable carboxymethylcellulose was chosen, as carrier, to allow niclosamide to induce proliferation of skeletal stem cells during the early stages of bone regeneration.

Additional dose response studies will be performed, using higher doses of niclosamide with longer healing time.

### Example 4. In Vitro Effects of Niclosamide on Skeletal Stem Cells of the Periodontium Of the Moue Incisor (Periodontal Ligament Stem Cells) Expressing Prx1

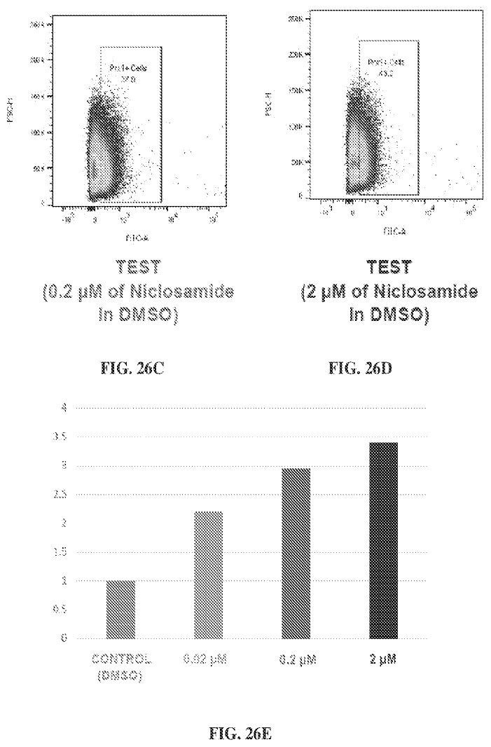

To test whether Niclosamide can induce proliferation of the skeletal stem cells of the periodontium (also known as periodontal ligament stem cells) cells were isolated from the periodontal ligament of the mouse incisors. The mouse incisor was chosen because, being an organ in continuous growth, it is particularly rich in stem cells. To perform these studies, transgenic mice was utilized expressing green fluorescent protein (GFP) under the Prx1 promoter. Thus, using these transgenic mice, quantification of GFP expressing cells by means of flow cytometry can be utilized to quantify Prx1 expressing cells. After 4 passages the periodontal-derived cells were treated with DMSO (vehicle, control) or Niclosamide dissolved in DMSO in various concentrations (0.02 M, 0.2 μM, and 2 μM) for 48 hours and quantitative analysis of the number of cells expressing Prx1 (and co-expressing GFP) was performed by means of flow cytometry (FIGS. 26A-26D). The data indicate that Prx1 expressing cells increase in number when exposed to Niclosamide, with more significant increases observed with higher doses of Niclosamide (FIG. 26E). Increases reached almost 3.5 folds with Niclosamide

at a concentration of 2 μM.

### Example 5. In Vivo Local Delivery of Niclosamide Absorbed into a Collagen Sponge

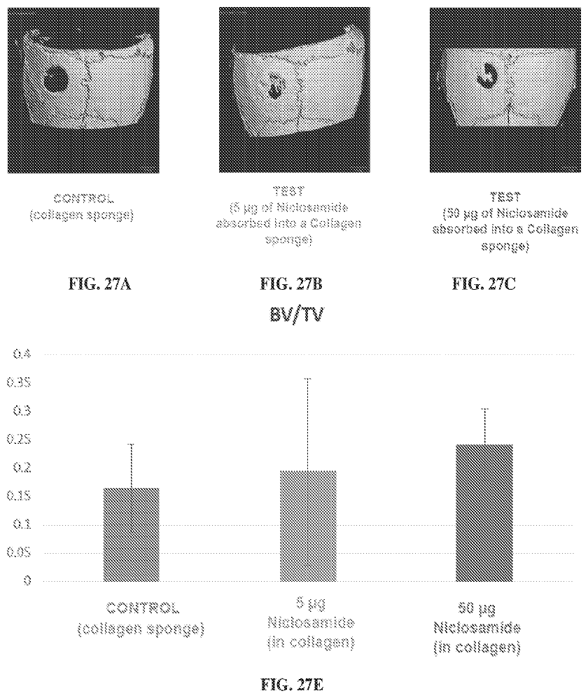

To test whether Niclosamide can induce bone regeneration when delivered by means of a collagen sponge instead of a methylcellulose gel, a critical size calvarial bone defect was created (2 mm in diameter) in the right parietal bone of skeletally mature C57Bl/6 female mice (8 week old mice). Immediately after creation of the defect, 5 μL of a 1 pg/pl solution of Niclosamide (dissolved in DMSO) or 5 μl of a 10 μg/μl solution of Niclosamide (dissolved in DMSO) were absorbed into a 1 mm×1 mm×1 mm collagen sponge. Then, the sponge carrying either 5 μg or 50 μg of Niclosamide was implanted into the calvarial bone defects (n=3). Bone defects of the control groups received collagen sponges absorbed with equal volumes (5 μl) of DMSO only Bone regeneration was assessed by means of μCT, 8 weeks after surgery (FIGS. 27A-27C) 3D rendering, (FIG. 27D) quantitative assessment of the regenerated Bone Volume (BV) within the Total Volume (TV) of the calvarial bone defects). While not statistically significant, this data show a trend of increasing of the regenerated bone mass when higher doses of Niclosamide are delivered. Lack of statistical significance is most probably due to the limited number of samples (n=3), not providing sufficient power to detect differences among the 3 groups. Importantly, since no adverse events were recorded during this study, this newly acquired data indicate that Niclosamide can also be effectively delivered by means of a collagen sponge without impairing bone regeneration or compromising the general health of the tested animals.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method of inducing proliferation of PRX1 expressing stem cells in a subject in need thereof, the method comprising:
administering an effective amount of niclosamide, or a pharmaceutically acceptable salt, prodrug, or derivative thereof.

2. The method of claim 1, wherein the method further comprises administering an effective amount of pyrvinium, or a pharmaceutically acceptable salt, prodrug, or derivative thereof.

3. The method of claim 1, wherein the method further comprises administering an effective amount of quercetin, or a pharmaceutically acceptable salt, prodrug, or derivative thereof.

4. The method of claim 1, wherein the PRX1 expressing stem cells are selected from skeletal stem cells (SSCs), pulp stem cells, periodontal stem cells, ligament stem cells, adipose stem cells, and dermis stem cells.

5. The method of claim 1, wherein the PRX1 expressing stem cells are skeletal stem cells (SSCs).

6. The method of claim 5, wherein inducing proliferation of skeletal stem cells (SSCs) is associated with bone regeneration.

7. The method of claim 5, wherein inducing proliferation of skeletal stem cells (SSCs) is associated with bone rejuvenation.

8. The method of claim 1, wherein the subject is a healthy subject.

9. The method of claim 1, wherein the subject has bone-related diseases, bone fracture, bone injury, bone abnormality, or any combination thereof.

10. The method of claim 9, wherein the subject has a bone-related disease.

11. The method of claim 1, wherein the bone related disease comprises osteoporosis, osteopenia, osteomalacia, rheumatoid arthritis, osteoarthritis, gouty arthritis, alveolar bone loss, osteotomy bone loss, systemic mastocytosis, adult hypophosphatasia, hyperadrenocorticism, osteogenesis imperfecta, Paget's disease, craniosynostosis, skeletal dysplasias, osteochondrodysplasias, osteogenesis imperfecta, Cushing's disease/syndrome, Tumer syndrome, Gaucher disease, Ehlers-Danlos syndrome, Marfan's syndrome, Menkes' syndrome, Fanconi's syndrome, hypercalcemia, hypocalcemia, arthritides, periodontal bone disease, childhood idiopathic bone loss, Paget's disease, scoliosis bone loss due to metastatic cancer, osteolytic lesions, rickets (including vitamin D dependent, type I and II, and x-linked hypophosphatemic rickets), fibrogenesis imperfecta ossium, osteosclerotic disorders such as pycnodysostosis and damage caused by macrophage-mediated inflammatory processes, or any combination thereof.

12. The method of claim 9, wherein the bone-related disease comprises osteoporosis, craniosynostosis, or periodontal bone related disease.

13. A method of inducing proliferation of skeletal stem cells (SSCs) in a subject in need thereof, the method comprising:
administering an effective amount of niclosamide, or a pharmaceutically acceptable salt, prodrug, or derivative thereof.

14-22. (canceled)

23. A method of treating a bone-related disease, bone fracture, bone injury, bone abnormality, or any combination thereof, or a skin related disease, the method comprising administering an effective amount of niclosamide, or a pharmaceutically acceptable salt, prodrug, or derivative thereof.

24-43. (canceled)

44. The method of claim 1, wherein the subject has a skin related disease thereof.

45. (canceled)

46. (canceled)

47. The method of claim 1, wherein the skin related disease comprises dermatis, atopic dermatis (e.g., eczema), rosacea, psoriasis, acne, or dermal wounds, or any combination thereof.

48-50. (canceled)

51. The method of claim 1, wherein administration comprises local administration to a bone affected by a bone related disease, a bone fracture, a bone injury, a bone abnormality, or any combination thereof.

52. The method of claim 1, further comprising administering niclosamide, or a pharmaceutically acceptable salt, prodrug, or derivative thereof and a pharmaceutically acceptable carrier.

53. The method of claim 1, wherein the carrier comprises carboxymethyl cellulose, collagen, collagen sponge, or any combination thereof.

54. (canceled)

55. (canceled)

56. The method of claim 1, wherein the PRX1 expressing stem cells comprise a PRX1 amino acid sequence comprising SEQ ID NO: 1, SEQ ID NO: 2, SEQ ID NO: 3, SEQ ID NO: 4, SEQ ID NO: 5, or SEQ ID NO: 6.
